(19) United States
(12) Patent Application Publication
(10) Pub. No.: US 2011/0008265 A1
(43) Pub. Date: Jan. 13, 2011

(54) In Situ Polymerization For Hair Treatment

(75) Inventors: Daniel Griffith Anderson, Sudbury, MA (US); David Thomas Puerta, Melrose, MA (US); Bryan Scott Akcasu, Burbank, CA (US); Mitchell John DeRosa, Hyde Park, MA (US); Amir Nashat, Newton, MA (US); Susan Alice Williams, Natick, MA (US); Richard Matthew Ramirez, Wichita Falls, TX (US); Susan Eilidh Bedford, Carisle, MA (US)

Correspondence Address: FITZPATRICK CELLA HARPER & SCINTO, 1290 Avenue of the Americas, NEW YORK, NY, 10104-3800, US

(73) Assignee: LIVING PROOF, INC., Cambridge, MA (US)

(21) Appl. No.: 12/706,401

(22) Filed: Feb. 16, 2010

Related U.S. Application Data
(62) Division of application No. 11/734,425, filed on Apr. 12, 2007, now abandoned.
(60) Provisional application No. 60/793,821, filed on Apr. 21, 2006.
(60) Provisional application No. 60/798,572, filed on May 8, 2006.
(60) Provisional application No. 60/800,143, filed on May 11, 2006.
(60) Provisional application No. 60/800,146, filed on May 11, 2006.
(60) Provisional application No. 60/853,612, filed on Oct. 23, 2006.

(51) Int. Cl. A61K 8/81; A61K 8/72; A61K 9/12; A61Q 5/06

(52) U.S. Cl. 424/43; 424/70.11; 424/70.16

(57) ABSTRACT

Hair care products represent a world-wide multi-billion dollar industry. Pre-formed polymers are commonly used in a variety of hair care products including shampoos, conditioners, gels, and hair sprays. The present invention provides technology for polymerizing monomers on hair in situ to produce desired hair characteristics. This eliminates the solubility and application issues found with some polymers. The polymerization of monomers on hair is typically initiated using a thermal or photoinitiated free radical initiator. In certain embodiments, the monomers are fluorinated thereby producing a fluorinated polymer on the hair upon polymerization. The invention provides monomers, initiators, methods, and kits for use in treating hair with polymers.

### RELATED APPLICATIONS

This application is a divisional of U.S. application Ser. No. 11/734,425, filed Apr. 12, 2007.

The present application claims priority under 35 U.S.C. §119(e) to U.S. provisional patent applications, U.S. Ser. No. 60/793,821, filed Apr. 21, 2006, U.S. Ser. No. 60/798,572, filed May 8, 2006; U.S. Ser. No. 60/800,143, filed May 11, 2006; U.S. Ser. No. 60/800,146, filed May 11, 2006; and U.S. Ser. No. 60/853,612, filed Oct. 23, 2006; the entire contents of each of which is incorporated herein by reference.

### BACKGROUND OF THE INVENTION

The hair care industry is a multi-billion dollar industry in the U.S. alone. The industry includes the development, production, and marketing of a large array of products for hair care, including shampoos, gels, mousses, lotions, sprays, conditioners, coloring products, and repair products. Most of these products utilize pre-formed polymers developed to impart a desired characteristic upon application to hair. For example, polymers are used to give hair shine, to style hair, to give hair a desired texture or feel, and to repair damaged hair. The current method of using pre-formed polymers in hair care involves applying a solution or mixture of the polymer in a solvent to the hair. After application of the polymer solution, the solvent evaporates leaving a film of the polymer on strands of the treated hair. The current method of dissolving or dispersing polymers in a solvent and applying those solutions to hair has limitations though. The size and other characteristics of these polymers presents problems, such as solubility, which pose significant hurdles to developing new hair care technologies.

In addition, existing hair care treatments suffer from numerous other limitations. One problem common to many hair care products is poor efficacy and longevity. For example, existing hair treatments are not robust and can lose their efficacy over the course of a day. Many treatments lose their efficacy upon exposure to water or excess humidity. In addition, many hair treatments weigh down hair, flake off, leave unsightly residues, fail to dry and set quickly, do no provide adequate hold, and are not effective for hard-to-treat hair (e.g., naturally curly hair). Treatments have been developed which overcome some of the issues; however, they typically involve permanently treating the hair with reducing and/or oxidizing agents which can damage hair. Thus, there remains a need for hair treatments that withstand the rigors of a typical user's daily routine and can maintain efficacy in a variety of environments without damaging hair fibers. In addition, hair care products which are designed to protect hair or deliver agents which improve hair strength, shine, color, and arrangement suffer from similar limitations as they also exhibit poor efficacy and longevity requiring daily application. It is preferable that a hair treatment be long lasting, not weigh down hair, not flake, and not leave any undesirable residues. Furthermore, the hair treatment should preferably dry and set relatively quickly, provide adequate hold, and be able to manage hard-to-treat hair.

### SUMMARY OF THE INVENTION

As described herein, it has been discovered that polymers generated via in situ polymerization on hair produce effects and characteristics desired by hair product consumers. Using the appropriate monomer with an optional polymerization initiator, a polymer can be created on the hair fiber upon application of light or heat. The resulting treatment is longer lasting than treatments based on pre-formed polymers and may resist humidity, washing away, and other daily treatments. Based on the in situ polymerization technique a whole new class of polymers can be used in hair care that could not be used before. For example, hydrophobic polymers that are difficult to solubilize in conventional hair care product formulations can now be used in hair treatments. Polymerization in situ on hair provides a treatment that is robust and is effective for longer periods of time and in more demanding environments than conventional hair care products formulated using pre-formed polymers. The inventive treatment may last from several days, to weeks, to months. In addition, it has been found that such polymers generated in situ on hair are able to improve hair strength, shine, color, elasticity, and optical properties.

The present invention relates to a system for the in situ polymerization of polymerizable monomers on hair. The treatment may be used to generate and/or preserve a particular hair style. The treatment may also be to enhance features of the treated hair. The present invention utilizes a novel method of polymerizing monomers directly on fibers of hair via a conditionally initiated in situ polyermization process. For example, the polymerization may be initiated by heat or light. The in situ polymerization process allows for the development and use of polymers that could not be used easily or effectively in hair treatment applications in the pre-formed state.

In one aspect, the invention provides a method for treating hair comprising applying to the hair of a subject at least one polymerizable monomer and at least one polymerization initiator, and initiating polymerization, thereby causing the polymerization of the polymerizable monomers on the hair. In certain embodiments, two or more different polymerizable monomers may be used in the treatment method. The polymerization is typically a free radical polymerization, which is heat initiated or photoinitiated. The type of initiation used may depend on the monomers and/or initiators being used in the treatment. The polymer may bond to the hair during the polymerization process. For example, the polymer may bond with the keratin or other biomolecules found in hair.

In certain embodiments, the invention provides a method for treating hair comprising applying to the hair of a subject a composition comprising at least one polymerizable monomer, at least one polymerization initiator, and, optionally, an acceptable solvent or other excipient (e.g., a physiologically, cosmetically, or pharmaceutically acceptable solvent or other excipient), and initiating polymerization, thereby causing the polymerization of the monomers on the hair. In certain embodiments, at least two different monomers are used. The monomers may be provide in the same or different compositions with or without a polymerization initiator. The composition containing monomer typically contains a polymerization initiator, though the initiator can also be applied in a separate treatment step. The composition(s) can be applied by soaking, rinsing, brushing, dipping, spraying, rubbing, etc. onto the subject's hair. In certain embodiments, the resulting polymer formed on the hair is resistant to humidity, washing, and other factors that lead to the removal or degradation of traditional hair product that contain pre-formed polymers. In certain embodiments, the monomers comprise about 0.1% to about 50% by weight of the composition. In certain embodiments, the monomers comprise about 0.1% to about 20% by weight. In certain embodiments, the monomers comprise about 0.5% to about 10% by weight. In certain embodiments, the monomers comprise about 0.5% to about 5% by weight. In certain embodiments, the monomers comprise about 1%, about 2%, about 3%, about 4%, or about 5% by weight of the composition. Typically, when the polymerization process is photoinitiated lower concentrations of the polymerizable monomer in the composition are needed, for example, from about 0.1% to about 5%. When the polymerization process is heat initiated, high concentrations of monomer may be used. In certain embodiments, the polymerizable monomers comprises up to about 50% of the composition for heat-activated polymerization processes. The concentration of monomer in the composition affects the overall strength and durability of the resulting polymer. Embodiments with high concentrations of monomer are effective in generating stronger polymers. Embodiments with lower concentrations of monomer are effective in generating polymers that are easier to manipulate. The polymerization initiator comprises about 0.1% to about 10% by weight, or about 0.5% to about 5% by weight of the composition. In certain embodiments, the polymerization initiator is about 1%, about 2%, about 3%, about 4%, or about 5% by weight. The solvent or other excipient then make up the remainder of the composition. The solvent or other excipients are typically about 95% to about 99% by weight of the composition. Suitable solvents include water, alcohols (e.g., denatured ethanol, ethanol, isopropanol), propylene glycol, ethylene glycol, and combinations thereof. The solvent may be a propellant such as difluoroethane or dimethyl ether. Preferably the components of the compositions are all biocompatible and do not cause undesired side effects such as inflammation, allergic reactions, etc. The compositions useful in treating hair in accordance with the present invention are also considered to be part of the present invention. For example, compositions comprising monomers, a polymerization initiator, and optionally, a suitable solvent or other excipient are provided by the present invention.

In certain embodiments, the polymerization initiator is activated by irradiation with light. In certain embodiments, the light used is IR, visible, or UV light. In certain embodiments, the UV light use has a wavelength of from about 200 nm to about 600 nm. In certain embodiments, the UV light has a wavelength of from about 200 nm to about 400 nm. In certain embodiments, the wavelength of the UV light is about 365 nm. In certain embodiments, the intensity of the light is from about 500 μW/cm² to about 10,000 μW/cm². In certain particular embodiments, the intensity of the light is about 7,000 μW/cm². The light may be applied to the hair as the monomer and initiator is being applied or subsequent to the application of the monomer and initiator to the hair. Treated hair is exposed to the appropriate light for about 10 seconds to about 1 minute, preferably, from about 20 seconds to about 40 seconds.

In certain other embodiments, the polymerization initiator is activated by exposing the hair to heat. The heat may be applied via a blow dryer, curling iron, flattening iron, heat lamps, hair dryer, or other devices suitable for delivering heat to hair. The temperatures needed to initiate heat range from about 30° C. to about 120° C. The output temperature of the heat source is typically in the range of about 50° C. to about 500° C. In certain embodiments, the output temperature of the heat source is from about 50° C. to about 200° C. Treated hair is exposed to the heat source for about 10 seconds to about 2 minutes, preferably, from about 20 seconds to about 60 seconds.

In certain embodiments, the polymerizable monomers used in the present invention include compounds with unsaturated functional groups (e.g., alkenes, alkynes, carbonyls), halogenated compounds, or other compounds with activated functional groups (e.g., epoxides). In certain embodiments, the monomer comprises a vinyl moiety, an acrylate or methacrylate moiety, a diene moiety, a maleimide moiety, or an epoxy moiety. Certain exemplary monomers useful in accordance with the present invention include ethyl acrylate, vinyl acrylate, 1,3-butanediol diacrylate, dipentaerythritol pentaacrylate, tridecyl methacrylate, styrene, and 3,4-epoxycyclohexylmethyl 3′,4′-epoxycyclohexane carboxylate. In certain embodiments, the monomer is a polybutadiene di(meth)acrylate oligomer. Various molecular weights of the oligomer may be used. In certain particular embodiments, the monomer is tricyclodecane dimethanol diacrylate. In other particular embodiments, the monomer is tricyclodecane dimethanol dimethacrylate.

In certain embodiments, the present invention provides a system for polymerizing fluorinated monomers on hair. Fluorinated monomers have been chosen for use in hair care due to the unique properties of the resulting fluorinated polymers. While pre-formed fluorinated polymers are not good candidates for traditional hair care products due to their low solubility and unfavorable surface tension, polymerization of fluorinated monomers on the hair surface overcomes these drawbacks and imparts unique and desirable properties to the hair. For example, the in situ polymerization of fluorinated monomers on hair results in hair with improved luster, smoothness and slip, static control, as well as a distinct feel. In certain embodiments, the invention provides a method for polymerizing fluorinated monomers on hair.

Any non-toxic fluorinated monomer suitable for polymerization may be used in the inventive hair treatment. Examples of suitable monomers include alkenes, alkynes, acrylates, methacrylates, fluoroacrylates, or other functional groups with an unsaturated functional group. The fluorinated monomer can include any number of fluorine atoms. In certain embodiments, the fluorinated monomer contains at least one fluorine atom. In certain other embodiments, the fluorinated monomer contains at least two, three, four, five, ten, fifteen, or twenty fluorine atoms. In certain embodiments, at least 10%, 25%, 30%, 40%, 50%, 60%, 75%, 80%, 90%, or 95% of the total number of hydrogen and fluorine atoms in the monomer are fluorine atoms. The monomer may also contain functional groups that are perfluorinated (e.g., an alkyl group). The fluorinated monomer may be mixed with unfluorinated monomers so that a co-polymer is formed upon polymerization.

In the inventive system, the polymerization initiator is typically oxygen-tolerant. In certain embodiments, the polymerization initiator is a free radical initiator. In other embodiments, the polymerization initiator is a thermal initiator. In certain embodiments, the free radical initiator is selected from the group consisting of benzophenone, benzyl dimethyl ketal, trimethylphosphine oxides, methyl thio phenyl morpholino ketones. In certain embodiments, the polymerization initiator is a cationic radical initiator such diaryliodonium and triarylsulfonium salts (e.g., benzoyl peroxide, 2,2′-azo-bis-isobutyrylnitrile (AIBN).

In a particular embodiment of the invention, the hair treatment system comprises the monomer pentaacrylate ester SR9041 (Sartomer), the polymerization initiator free radical photoinitiator KT046 (Sartomer), and a solvent mixture of propylene glycol and denatured ethanol. In certain embodiments, the components of the composition are as follows: SR9041 at about 1% by weight; KT046 at about 1% by weight; propylene glycol at 2% by weight; and denatured ethanol at 96% by weight.

In a particular embodiment of the invention, the hair treatment system comprises the monomer trimethylolpropane triacrylate, the thermal polymerization initiator benzoyl peroxide, and the solvent denatured ethanol. In certain embodiments, the components of the composition are as follows: trimethylolpropane triacrylate at about 0.5-50% by weight; benzoyl peroxide at about 0.1-2% by weight; and denatured ethanol at 48-99.4% by weight.

The polymerization process is performed under conditions suitable to yield the desired properties of the resulting polymer. For example, the extent of polymerization or cross-linking may be controlled by the time of the reaction, the amount/concentration of initiator, the polymer starting material, the initiator, the frequency of the light used, additives, temperature of the reaction, solvent used, concentration of polymer starting material, oxygen inhibition, water or solvent inhibition, etc.

The inventive polymer system can be used in a variety of hair care treatments. The inventive treatment may affect the color, condition, style, strength, shine, elasticity, and optical properties of the treated hair. The inventive system can improve the luster of treated hair, improve smoothness and slip, improve static control, and/or provide a unique feel. The inventive system may also be used to straighten wavy, curly, or frizzy hair. The inventive system can alternatively be used to curl or style hair. The inventive system can also be used to treat damaged hair.

In another aspect, the invention provides kits for treating hair based on polymerizing monomers on hair in situ. The kit typically contains all the materials needed for treating hair using the inventive system. Materials in the kit may include all or some of the following: monomer(s) (e.g., fluorinated monomers, non-fluorinated monomers), polymerization initiator, solvent, excipients, water, applicator, spray bottle, brush, light source, heat source, blow dryer, curling iron, instructions for use, etc. In certain embodiments, the kit includes the monomers needed for the hair treatment, the polymerization initiator, and the solvent or other acceptable excipients useful in the inventive hair treatment system. The kit may include the materials conveniently packaged for use in a hair stylist's shop or for home use. The kit typically includes instructions for teaching one how to use the components of the kit in treating hair. The kit may include the materials needed for a single use or for multiple uses.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

It will be appreciated that the polymers, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term acyl as used herein refers to a group having the general formula —C(═O)R, where R is alkyl, alkenyl, alkynyl, aryl, carbocylic, heterocyclic, or aromatic heterocyclic. An example of an acyl group is acetyl.

The term aliphatic, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

The term alkyl as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-10 carbon atoms. In another embodiment, the alkyl group employed contains 1-8 carbon atoms. In still other embodiments, the alkyl group contains 1-6 carbon atoms. In yet another embodiments, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents.

The term alkoxy as used herein refers to a saturated (i.e., alkyl-O—) or unsaturated (i.e., alkenyl-O— and alkynyl-O—) group attached to the parent molecular moiety through an oxygen atom. In certain embodiments, the alkyl group contains 1-20 aliphatic carbon atoms. In certain other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples include, but are not limited to, methoxy, ethoxy, propoxy, isopropoxy, n-butoxy, tert-butoxy, i-butoxy, sec-butoxy, neopentoxy, n-hexoxy, and the like.

The term alkenyl denotes a monovalent group derived from a hydrocarbon moiety having at least one carbon-carbon double bond by the removal of a single hydrogen atom. In certain embodiments, the alkenyl group employed in the invention contains 1-20 carbon atoms. In some embodiments, the alkenyl group employed in the invention contains 1-10 carbon atoms. In another embodiment, the alkenyl group employed contains 1-8 carbon atoms. In still other embodiments, the alkenyl group contains 1-6 carbon atoms. In yet another embodiments, the alkenyl group contains 1-4 carbons. Alkenyl groups include, for example, ethenyl, propenyl, butenyl, 1-methyl-2-buten-1-yl, and the like.

The term alkynyl as used herein refers to a monovalent group derived form a hydrocarbon having at least one carbon-carbon triple bond by the removal of a single hydrogen atom. In certain embodiments, the alkynyl group employed in the invention contains 1-20 carbon atoms. In some embodiments, the alkynyl group employed in the invention contains 1-10 carbon atoms. In another embodiment, the alkynyl group employed contains 1-8 carbon atoms. In still other embodiments, the alkynyl group contains 1-6 carbon atoms. Representative alkynyl groups include, but are not limited to, ethynyl, 2-propynyl (propargyl), 1-propynyl, and the like.

The term alkylamino, dialkylamino, and trialkylamino as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. In certain embodiments, the alkyl group contain 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contain 1-6 aliphatic carbon atoms. In yet other embodiments, the alkyl group contain 1-4 aliphatic carbon atoms. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of thioalkoxyl moieties include, but are not limited to, methylthio, ethylthio, propylthio, isopropylthio, n-butylthio, and the like.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms aryl and heteroaryl, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, aryl refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term heteroaryl, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂, —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term carboxylic acid as used herein refers to a group of formula —CO₂H.

The terms halo and halogen as used herein refer to an atom selected from fluorine, chlorine, bromine, and iodine.

The term haloalkyl denotes an alkyl group, as defined above, having one, two, or three halogen atoms attached thereto and is exemplified by such groups as chloromethyl, bromoethyl, trifluoromethyl, and the like.

The term heteroaliphatic, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term heterocyclic, as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term carbamoyl, as used herein, refers to an amide group of the formula —CONH₂.

The term carbonyldioxyl, as used herein, refers to a carbonate group of the formula —O—CO—OR.

The term hydrocarbon, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The term thiohydroxyl or thiol, as used herein, refers to a group of the formula —SH.

The following are more general terms used throughout the present application:

As used herein, the singular forms “a”, “an”, and “the” include the plural reference unless the context clearly indicates otherwise. Thus, for example, a reference to “a monomer” includes a plurality of such monomers.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a domesticated animal. In certain embodiments, the animal is human. An animal may be a transgenic animal.

“Biocompatible”: The term “biocompatible”, as used herein is intended to describe compounds that are not toxic to cells. Compounds are “biocompatible” if their addition to cells in vitro results in less than or equal to 20% cell death. The administration in vivo does not cause inflammation, cancer, birth defects, neurotoxicity, or other such adverse side effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably does not cause inflammation, cancer, birth defects, neurotoxicity, or other such adverse side effects in vivo. In certain preferred embodiments, the chemical reactions relied upon to break down the biodegradable compounds are uncatalyzed. For example, the inventive materials may be broken down in part by the hydrolysis of the ester bonds found in cross-linked material.

“Keratin”: The term “keratin” as used herein refers any one of a class of fibrous structural proteins found in hair, wool, and nails. Keratin proteins contains a large quantity of cysteine residues. Human hair is approximately 15% cysteine residues cross-linked by disulfide bridges. The helical keratin molecules twist around each other to form elongated strands call intermediate filaments.

“Monomer”: As used herein, a “monomer” is a chemical compound that is linked to other monomers covalently to form a polymer. Examples of monomers include acrylates, methacrylates, epoxide containing compounds, styrenes, and vinyl alcohol. In certain embodiments, the monomers useful in accordance with the present invention are susceptible to free radical polymerization.

“Oligomer”: The term “oligomer,” as used herein, refers to a chemical compound with a finite number of structural units connected by covalent bonds. An oligomer has less monomeric units than the corresponding polymer. An oligomer typically has between 3 to 100 monomeric units making up its structure. In certain embodiments, less than 10 monomeric units are found in the oligomer. In certain embodiments, less than 20 monomeric units are found in the oligomer. In certain embodiments, less than 50 monomeric units are found in the oligomer. In certain embodiments, less than 100 monomeric units are found in the oligomer.

“Peptide” or “protein”: As used herein, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polymer”: The term “polymer,” as used herein, refers to a chemical compound of repeating structural units (monomers) connected by covalent bonds. A polymer is typically of high molecular weight and may comprise 10s to 100s to 1000s or even more monomers. In certain embodiments, the polymer comprises at least 10 monomeric units linked covalently together. In certain embodiments, the polymer may be a co-polymer comprising different types of polymers. The polymer may be cross-linked or uncross-linked. The polymer may be linear or branched. In certain embodiments, the polymer is formed by in situ polymerization on hair.

### BRIEF DESCRIPTION OF THE DRAWING

FIG. 1. Structures of tricyclodecane dimethanol diacrylate (TCDDA) (Sartomer SR833S) (top) and tricyclodecane dimethanol dimethacrylate (TCDDMA) (bottom).

FIG. 2. Curl factor number (CF#) of varying concentrations of TCDDA (Sartomer SR833S) (0.5-8%) combined with benzoyl peroxide (1%) in an ethanol/water solution compared to a commercial styling product. Increasing CF# indicates increased humidity resistance.

FIG. 3. Curl factor number (CF#) of varying concentrations of TCDDMA (4-8%) combined with benzoyl peroxide (1%) in an ethanol/water solution compared to a commercial styling product. Increasing CF# indicates increased humidity resistance.

FIG. 4. Acrylate-modified Polybutadiene (left) and poly(isochloroprene) (right) monomers.

FIG. 5. Humidity resistance of Polybutadiene Di(meth)acrylate derivatives at 90% RH over 120 minutes. Curl factor number (CF#) of varying versions of Polybutadiene Di(meth)acrylate (CN301, CN303, CN307) (8%) combined with benzoyl peroxide (1%) and AIBN (1.2%) in ethyl acetate compared to a commercial styling product. Increasing CF# indicates increased humidity resistance.

FIG. 6. Curl drop out results after 120 minutes at 90% RH. From left to right: Commercial Product, 2%, 4%, and 8% Polybutadiene Dimethacrylate (CN303) with 2% BPO and 1.2% AIBN in ethyl acetate.

FIG. 7. Humidity resistance of Polybutadiene Diacrylate (BAC-15) and poly(isoprene)diacrylate at 90% RH over 120 minutes. Curl factor # (CF#) of the two components at 4% combined with benzoyl peroxide (2%) and AIBN (1.2%) in an ethyl acetate solution compared to a commercial styling product. Increasing CF# indicates increased humidity resistance.

FIG. 8A-C. Chemical diagrams and names of fluorinated monomers for hair treatment.

FIG. 9. Curly/Frizzy Brown Hair, untreated.

FIG. 10. Curly, frizzy hair treated with from left to right: Phyto Defrisant, A1 (4% 3-(perfluoro-5-methylhexyl)-2-hydroxypropyl methacrylate) with 0.5% benzoyl peroxide in ethanol, A2 (8% 3-(perfluoro-5-methylhexyl)-2-hydroxypropyl methacrylate) with 0.5% benzoyl peroxide in ethanol, and A3 (12% 3-(perfluoro-5-methylhexyl)-2-hydroxypropyl methacrylate) with 0.5% benzoyl peroxide in ethanol. All hair tresses were styled in the same manner: soaked with formulation, blow dried straight and flat ironed straight.

FIG. 11. Samples from FIG. 3 after 45 minutes at 85% relative humidity and 37° C.

FIG. 12. Hair samples after static resistance test. Water as control (A), inventive formulation (2,2,3,3,4,4,5,5-octafluoro-1,6-hexyl dimethacrylate/dibenzoyl peroxide) (B), Bumble & Bumble Styling Lotion (C), and Phyto Defrisant (D).

### DETAILED DESCRIPTION OF CERTAIN PREFERRED EMBODIMENTS OF THE INVENTION

The present invention provides a system for the in situ polymerization of monomers (e.g., acrylates, methacrylates, dienes, maleimides, fluorinated monomers) on hair. It has been discovered that the application of polymerizable monomers and a polymerization initiator to hair followed by initiation of polymerization leads to the formation of polymers on the treated hair. The polymerization of monomers on hair during styling or treatment has been shown to improve luster, smoothness and slip, and static control while imparting a distinct feel to the treated hair. The inventive system can also be used to affect the color, condition, style, strength, shine, elasticity, and optical properties of the treated hair. The inventive treatment is robust and long-lasting resisting removal and/or degradation by humidity, washing, other factors.

One advantage of the present system is that certain polymers can not effectively be applied to hair via traditional means using pre-formed polymers given their low solubility. In the inventive system, polymerizable monomers are applied to hair with a polymerization initiator, and the treated hair is then exposed to light or heat to cause the polymerization of the monomers in situ on the hair. Thus, the inventive system eliminates the need to solubilize or formulate polymers with low solubility. Polymers that could not before be used on hair can now be prepared directly on strands of hair. The polymers may be homopolymers with repeating units of the same type or heteropolymers with repeating units of two or more different types. In situ polymerization gives the user greater control and flexibility in styling hair. The invention provides methods, compositions, kits, and materials for treating hair using the inventive system.

### Polymerizable Monomers

A variety of polymerizable monomers may be used in accordance with the present invention to generate polymers in situ on strands of hair. Some monomers generate polymers that are only available for hair treatment using the inventive in situ polymerization technique. Different monomers or combinations of monomers may be used to create polymers with different properties, thereby creating different cosmetic effects. The availability of a wide range of monomers for polymer generation also allows for the development of polymers with a wide variety of properties which include longevity, hold strength, optical properties, feel, color, texture, shape preservation, etc.

A polymerizable monomer is any chemical compound (e.g., organic compound), regardless of molecular weight, that when exposed to a polymerization initiator reacts with other monomers to generate a polymers. In certain embodiments, the monomers are monomers in the strict sense of the term in that the monomer does not include a repeating unit. That is, the monomer is not an oligomer or low molecular weight polymer. In certain embodiments, the monomers are oligomers, resins, partially polymerized polymers, low molecular weight polymers, or uncross-linked polymers. In certain embodiments, the oligomers are of various molecular weights and may contain 2-50 monomer units. In certain embodiments, the oligomer contains 2-10 monomer units. In certain embodiments, the oligomer contains 2-20 monomer units.

In certain embodiments, the molecular weight of the monomer is less than about 2,000 g/mol. In certain other embodiments, the molecular weight of the monomer is less than about 1,500 g/mol. In certain other embodiments, the molecular weight of the monomer is less than about 1,000 g/mol. In certain embodiments, the molecular weight of the monomer is less than about 500 g/mol. In certain embodiments, the molecular weight of the monomer is less than about 400 g/mol. In certain embodiments, where monomer toxicity is an issue, monomers with higher molecular weights are preferred so as to decrease the ability of the monomer to pass through the skin. In such embodiments, the molecular weight of the monomer is greater than 500 g/mol. In such embodiments, the molecular weight of the monomer is greater than 1,000 g/mol. In such embodiments, the molecular weight of the monomer is greater than 1,500 g/mol. In certain embodiments, the molecular weight of the monomer is greater than 2,000 g/mol. In certain embodiments, the molecular weight of the monomer is greater than 2,500 g/mol. In certain embodiments, the molecular weight of the monomer is greater than 3,500 g/mol. In certain embodiments, the molecular weight of the monomer is greater than 5,000 g/mol. In certain embodiments, the molecular weight of the monomer is greater than 10,000 g/mol.

The polymerizable monomer comprises a functional group suitable for polymerization. Any functional group that can be polymerized using a free radical or ionic polymerization reaction can be used. In certain embodiments, the monomers include a functional group with at least one degree of unsaturation. For example, the monomer includes a double bond or triple bond. Exemplary functional groups suitable for polymerization include alkenes, alkynes, carbonyls, imines, thiocarbonyls, acrylates, methacrylates, acrylates, crotonates, styrenes, nitriles, cyano, vinyl, styrene, crotonate, cinnamate, dienes, trienes, eneynes, maleimides, etc. In certain particular embodiments, the monomers comprise a vinyl group. In certain particular embodiments, the monomers comprise an acrylate functional group. In certain particular embodiments, the monomers comprise a methacrylate functional group. In certain particular embodiments, the monomers comprise a diene moiety. In certain embodiments, the monomers comprise a conjugated diene moiety. In certain embodiments, the monomers comprise a maleimide moiety. Other reactive functional groups may also be used including epoxides and halogen-containing compounds.

In certain embodiments, the monomer is an alkene. In certain particular embodiments, the alkene is monosubstituted. In other embodiments, the alkene is disubstituted. Disubstituted alkenes may be either in the cis or trans configuration or a mixture thereof. In yet other embodiments, the alkene is trisubstituted. The trisubstituted alkene may be in either the E or Z configuration or a mixture thereof. In still other embodiments, the alkene is tetrasubstituted. Again, various isomers are possible and are considered part of this invention. In certain embodiments, the monomer is an alkyne.

In certain embodiments, the monosubstituted monomer is of the formula:

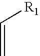

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, R₁ is a substituted or unsubstituted, branched or unbranched aliphatic moiety. In certain embodiments, R₁ is an alkyl moiety. In certain embodiments, R₁ is of one of the formulae:

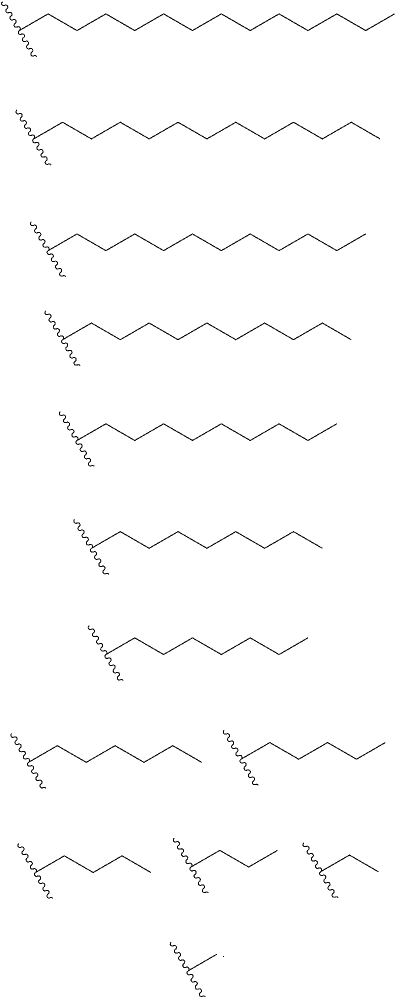

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

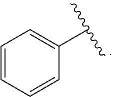

In certain particular embodiments, R₁ is of the formula:

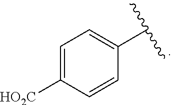

In certain particular embodiments, R₁ is a substituted or unsubstituted phenyl moiety. In certain embodiments; R₁ is substituted phenyl (e.g., a phenyl ring with 1, 2, 3, 4, or 5 substituents). In other embodiments, R₁ is a substituted or unsubstituted heteroaryl moiety. In certain embodiments, R₁ is —C(═O)R_{A}. In other embodiments, R₁ is —CO₂R_{A}. In certain particular embodiments, R₁ is —CO₂R_{A}, wherein R_{A} is one of the formulae:

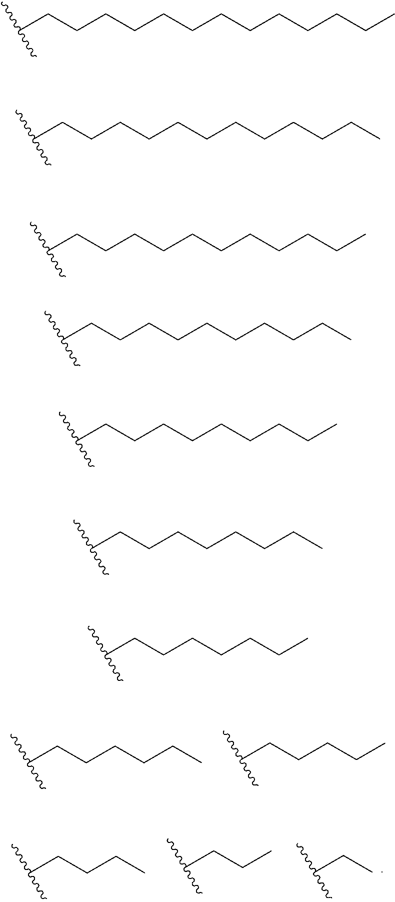

As would be appreciated by one of skill in this art, any of the above alkyl groups may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

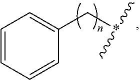

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

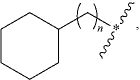

wherein n is an integer between 0 and 12, inclusive. In certain embodiments, n is 0. In certain embodiments, n is 1. In other embodiments, n is 2, 3, 4, 5, or 6.

In certain embodiments, the monomer is a acrylate of formula:

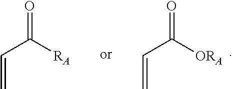

Exemplary acrylate monomers include:

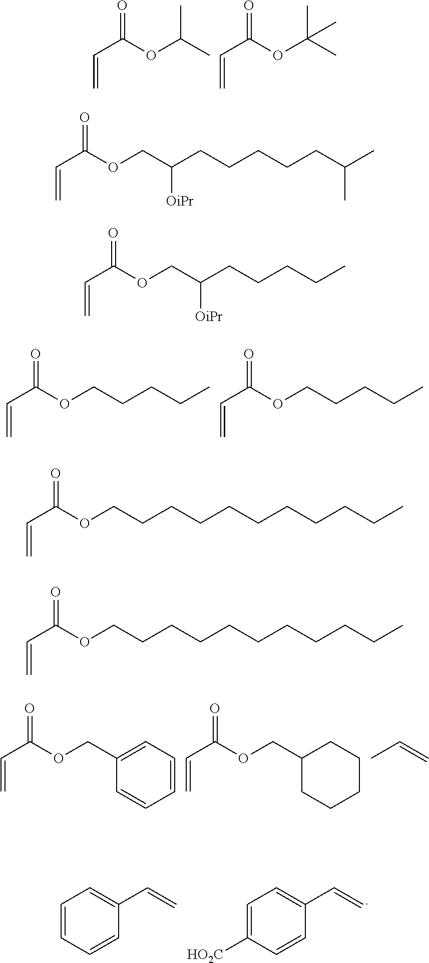

In certain embodiments, the disubstituted monomer is of one of the formulae:

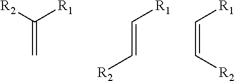

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. R₁ and R₂ may form a cyclic structure, for example, a maleimide moiety.

In other embodiments, R₁ is a substituted or unsubstituted, branched or unbranched aliphatic moiety. In certain embodiments, R₁ is a alkyl moiety. In certain embodiments, R₁ is of one of the formulae:

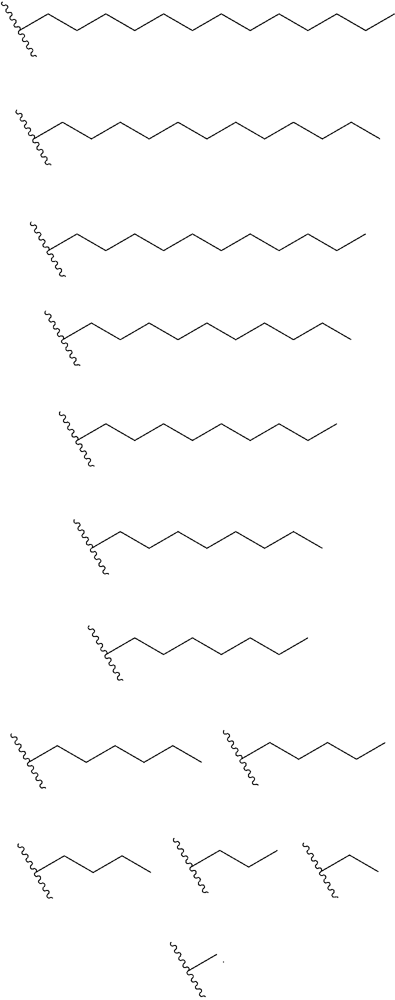

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

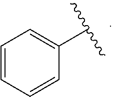

In certain particular embodiments, R₁ is of the formula:

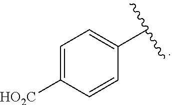

In certain particular embodiments, R₁ is a substituted or unsubstituted phenyl moiety. In certain embodiments, R₁ is substituted phenyl moiety (e.g., a phenyl ring with 1, 2, 3, 4, or 5 substituents). In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain embodiments, R_{A} is

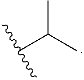

In other embodiments, R_{A} is t-butyl. In certain particular embodiments, R₁ is —CO₂R_{A}, wherein R_{A} is one of the formulae:

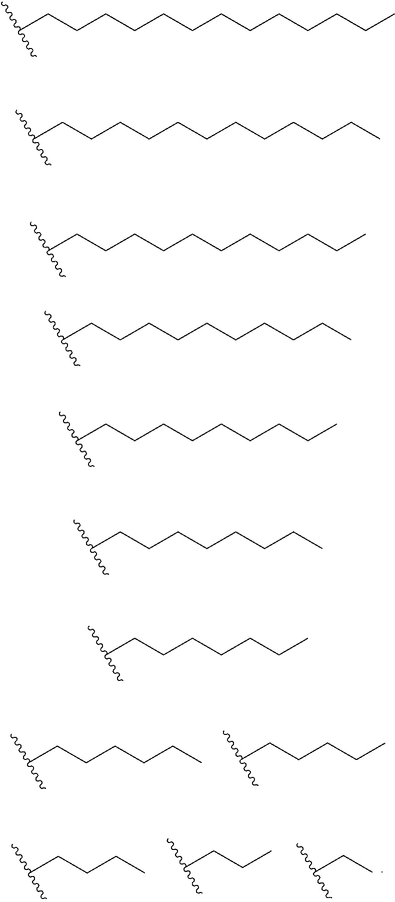

As would be appreciated by one of skill in this art, any of the above alkyl groups may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

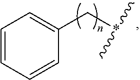

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

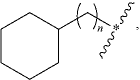

wherein n is an integer between 0 and 12, inclusive. In certain embodiments, n is 0. In certain embodiments, n is 1. In other embodiments, n is 2, 3, 4, 5, or 6.

In other embodiments, R₂ is substituted or unsubstituted, branched or unbranched aliphatic. In yet other embodiments, R₂ is C₁-C₆ alkyl. In certain embodiments, R₂ is a alkyl moiety. In certain particular embodiments, R₂ is methyl. In certain embodiments, R₂ is a aryl or heteroaryl moiety. In certain embodiments, R₂ is a phenyl moiety. In certain particular embodiments, R₂ is a phenyl moiety.

In certain embodiments, R₁ is —CO₂R_{A}. In other embodiments, R₁ is —CO₂R_{A}, and

R₂ is C₁-C₆ alkyl. In other embodiments, R₁ is —CO₂R_{A}, and R₂ is methyl.

In certain embodiments, the monomer is a methacrylate of formula:

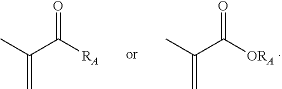

In certain embodiments, the monomer is a crotonate of formula:

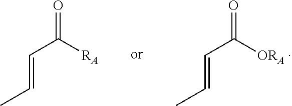

Exemplary disubstituted fluorinated monomers include:

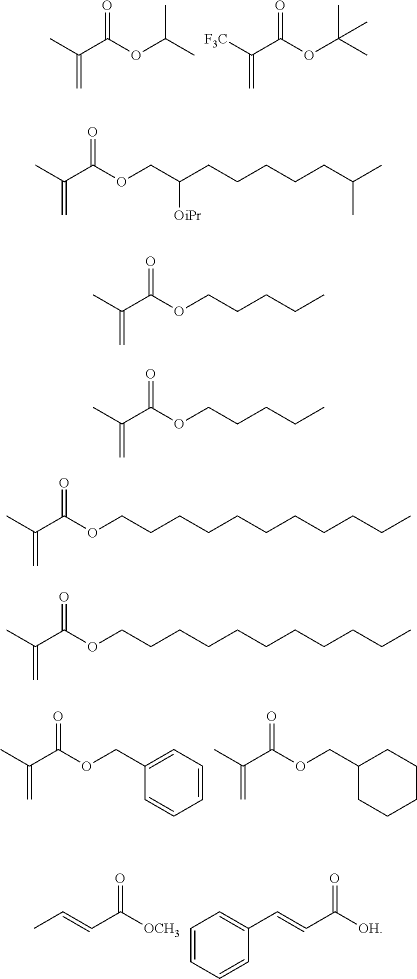

In certain embodiments, the trisubstituted fluorinated monomer is of one of the formulae:

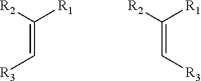

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. R₁, R₂, and/or R₃ may form a cyclic structure.

In other embodiments, R₁ is a substituted or unsubstituted, branched or unbranched aliphatic moiety. In certain embodiments, R₁ is a alkyl moiety. In certain embodiments, R₁ is of one of the formulae:

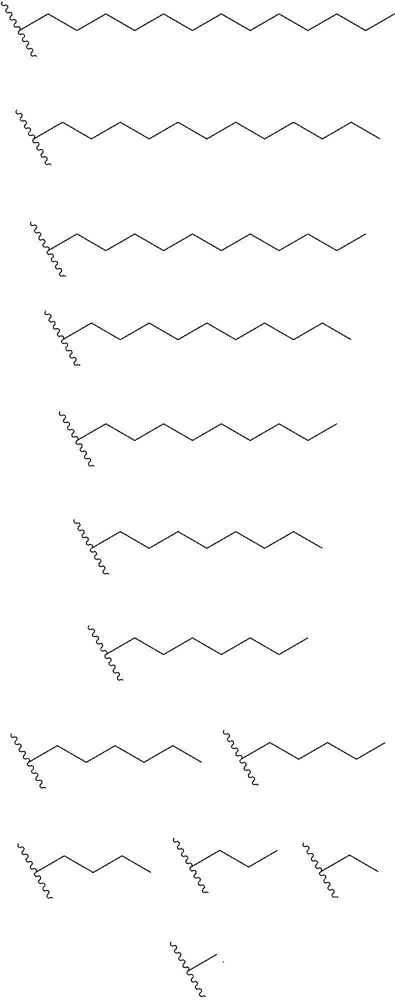

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

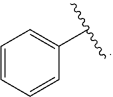

In certain particular embodiments, R₁ is of the formula:

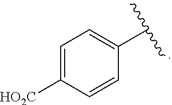

In certain particular embodiments, R₁ is a substituted or unsubstituted phenyl moiety. In certain embodiments, R₁ is substituted phenyl moiety (e.g., a phenyl ring with 1, 2, 3, 4, or 5 substituents). In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain embodiments, R_{A} is

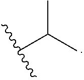

In other embodiments, R_{A} is t-butyl. In certain particular embodiments, R₁ is —CO₂R_{A}, wherein R_{A} is one of the formulae:

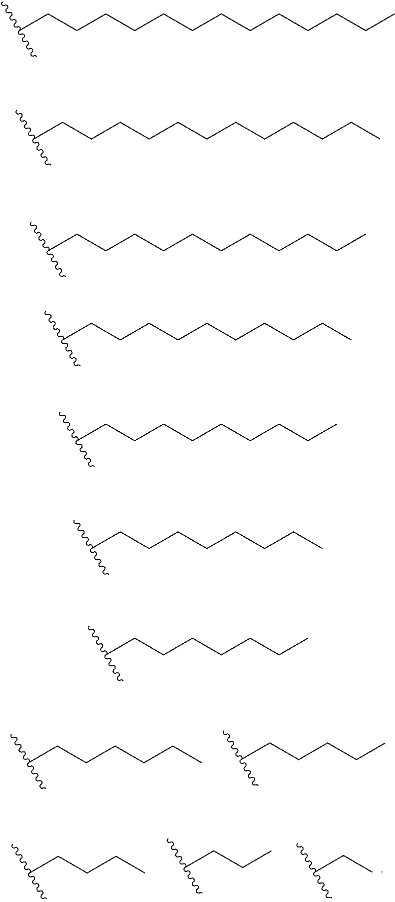

As would be appreciated by one of skill in this art, any of the above alkyl group may be partially substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

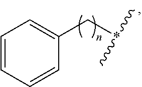

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

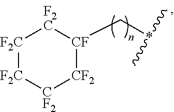

wherein n is an integer between 0 and 12, inclusive. In certain embodiments, n is 0. In certain embodiments, n is 1. In other embodiments, n is 2, 3, 4, 5, or 6.

In other embodiments, R₂ is substituted or unsubstituted, branched or unbranched aliphatic. In yet other embodiments, R₂ is C₁-C₆ alkyl. In certain particular embodiments, R₂ is methyl.

In certain embodiments, R₂ is of one of the formulae:

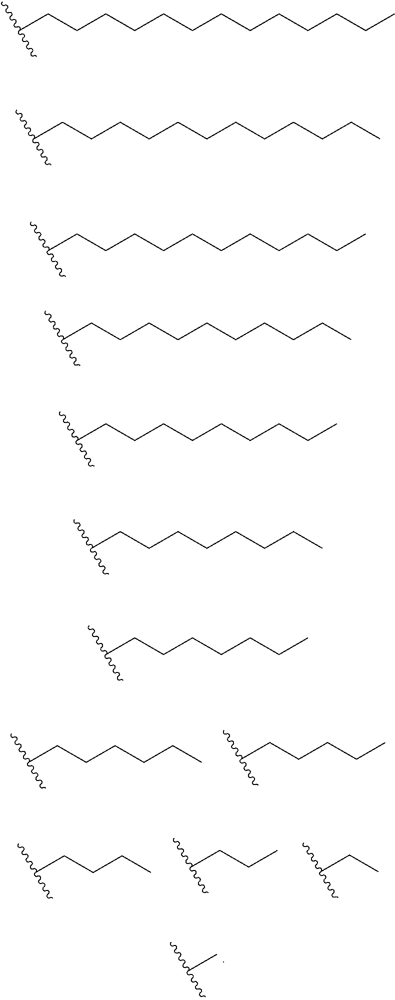

As would be appreciated by one of skill in this art, any of the above alkyl group may be substituted, branched, unsaturated, and/or cyclic. In yet other embodiments, R₂ is a substituted or unsubstituted, branched or unbranched heteroaliphatic moiety. In still other embodiments, R₂ is a substituted or unsubstituted acyl moiety. In other embodiments, R₂ is a substituted or unsubstituted aryl moiety. In certain particular embodiments, R₂ is of the formula:

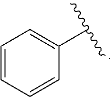

In certain particular embodiments, R₂ is of the formula:

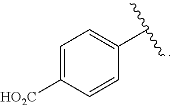

In certain particular embodiments, R₂ is a substituted or unsubstituted phenyl moiety. In certain embodiments, R₂ is substituted phenyl moiety (e.g., a phenyl ring with 1, 2, 3, 4, or 5 substituents). In other embodiments, R₂ is a substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain embodiments, R_{B} is

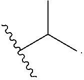

In other embodiments, R_{B} is t-butyl. In certain particular embodiments, R₂ is —CO₂R_{B}, wherein R_{B} is one of the formulae:

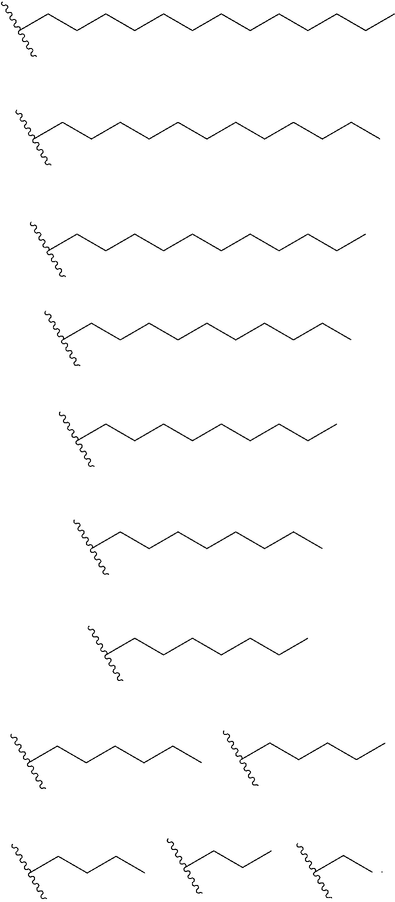

As would be appreciated by one of skill in this art, any of the above alkyl groups may be substituted, branched, unsaturated, and/or cyclic. In certain particular embodiments, R₂ is —CO₂R_{B}, wherein R_{B} is aryl or arylalkyl. In certain particular embodiments, R₂ is —CO₂R_{B}, wherein R_{B} is of the formula:

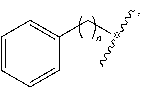

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

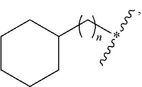

wherein n is an integer between 0 and 12, inclusive. In certain embodiments, n is 0. In certain embodiments, n is 1. In other embodiments, n is 2, 3, 4, 5, or 6.

In other embodiments, R₃ is substituted or unsubstituted, branched or unbranched aliphatic. In yet other embodiments, R₃ is C₁-C₆ alkyl. In certain particular embodiments, R₃ is methyl.

In certain embodiments. R₃ is of one of the formulae:

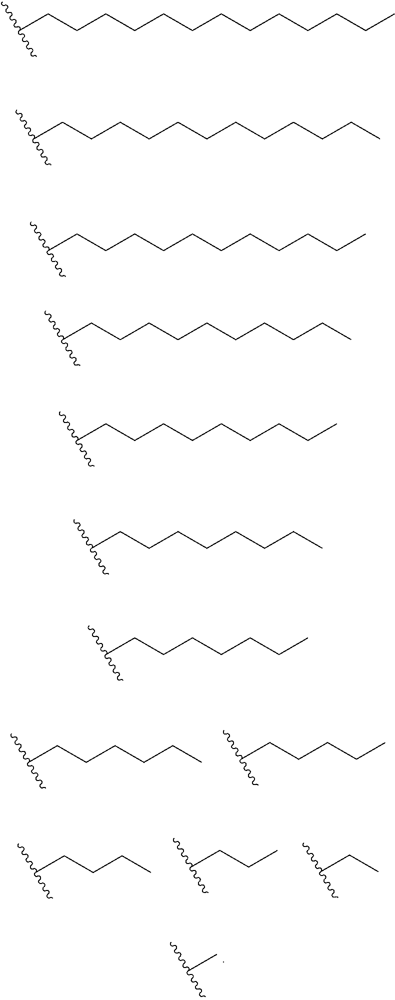

As would be appreciated by one of skill in this art, any of the above alkyl groups may be substituted, branched, unsaturated, and/or cyclic. In yet other embodiments, R₃ is a substituted or unsubstituted, branched or unbranched heteroaliphatic moiety. In still other embodiments, R₃ is a substituted or unsubstituted acyl moiety. In other embodiments, R₃ is a substituted or unsubstituted aryl moiety. In certain particular embodiments, R₃ is of the formula:

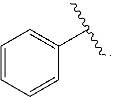

In certain particular embodiments, R₃ is of the formula:

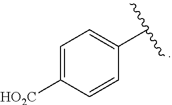

In certain particular embodiments, R₃ is a substituted or unsubstituted phenyl moiety. In certain embodiments, R₃ is a substituted phenyl moiety (e.g., a phenyl ring with 1, 2, 3, 4, or 5 substituents). In other embodiments, R₃ is a substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain embodiments, R_{C} is

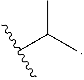

In other embodiments, R_{C} is t-butyl. In certain particular embodiments, R₃ is —CO₂R_{A}, wherein R_{C} is one of the formulae:

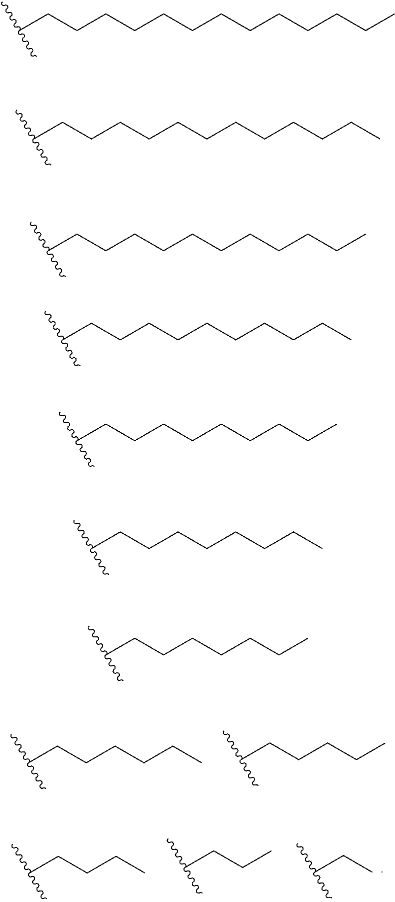

As would be appreciated by one of skill in this art, any of the above alkyl groups may be substituted, branched, unsaturated, and/or cyclic. In certain particular embodiments, R₃ is —CO₂R_{C}, wherein R_{C} is aryl or arylalkyl. In certain particular embodiments, R₃ is —CO₂R_{C}, wherein R_{C} is of the formula:

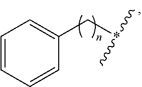

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

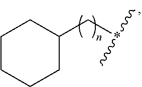

wherein n is an integer between 0 and 12, inclusive. In certain embodiments, n is 0. In certain embodiments, n is 1. In other embodiments, n is 2, 3, 4, 5, or 6.

In other embodiments, R₁ is —CO₂R_{A}, and R₂ and R₃ are both methyl.

Examplary trisubstituted fluorinated monomers include:

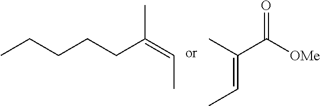

In certain embodiments, the tetrasubstituted fluorinated monomer is of one of the formulae:

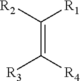

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂, —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{A}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —C(═O)N(R_{D})₂; —CN; —SCN; —SR_{D}; —SOR_{D}; —SO₂R_{D}; —NO_{D}; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. R₁, R₂, R₃, and/or R₄ may form a cyclic structure.

In other embodiments, R₁ is a substituted or unsubstituted, branched or unbranched aliphatic moiety. In certain embodiments, R₁ is of one of the formulae:

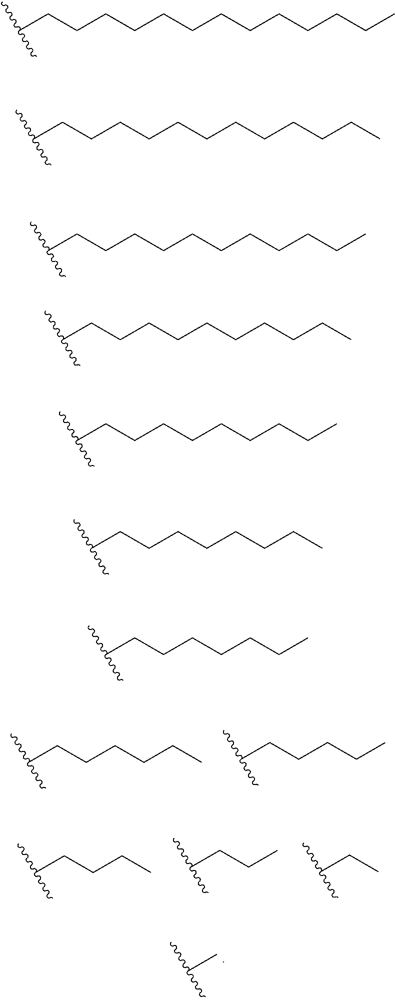

As would be appreciated by one of skill in this art, any of the above alkyl groups may be partially substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

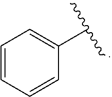

In certain particular embodiments, R₁ is of the formula:

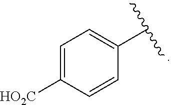

In certain particular embodiments, R₁ is a substituted or unsubstituted phenyl moiety. In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl.

In certain particular embodiments, R_{A} is methyl. In certain embodiments, R_{A} is

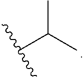

In other embodiments, R_{A} is t-butyl. In certain particular embodiments, R₁ is —CO₂R_{A}, wherein R_{A} is one of the formulae:

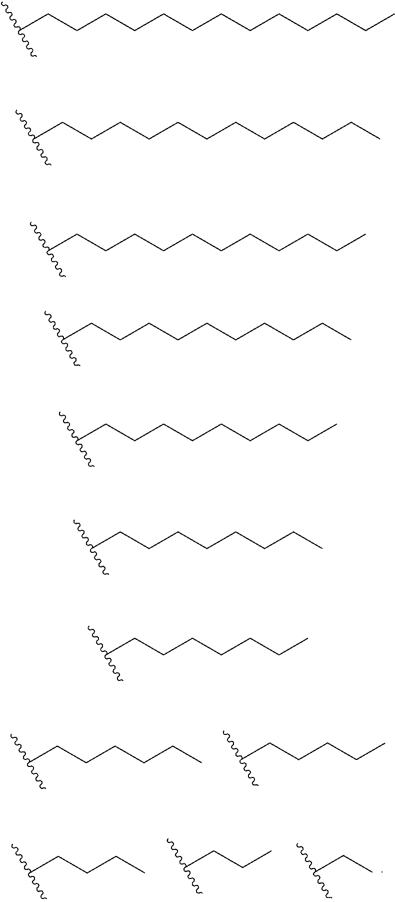

As would be appreciated by one of skill in this art, any of the above alkyl groups may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

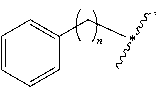

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

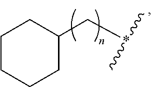

wherein n is an integer between 0 and 12, inclusive. In certain embodiments, n is 0. In certain embodiments, n is 1. In other embodiments, n is 2, 3, 4, 5, or 6.

In other embodiments, R₂ is substituted or unsubstituted, branched or unbranched aliphatic. In yet other embodiments, R₂ is C₁-C₆ alkyl. In certain particular embodiments, R₂ is methyl.

In certain embodiments, R₂ is of one of the formulae:

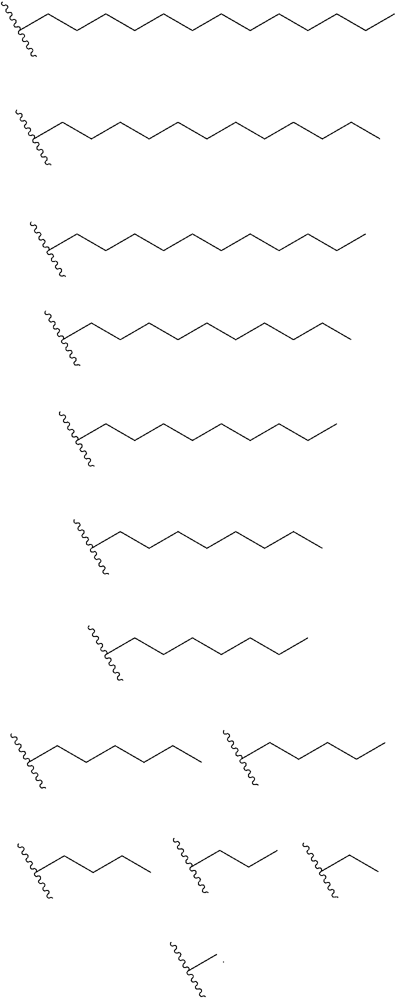

As would be appreciated by one of skill in this art, any of the above alkyl groups may be substituted, branched, unsaturated, and/or cyclic. In yet other embodiments, R₂ is a substituted or unsubstituted, branched or unbranched heteroaliphatic moiety. In still other embodiments, R₂ is a substituted or unsubstituted acyl moiety. In other embodiments, R₂ is a substituted or unsubstituted aryl moiety. In certain particular embodiments, R₂ is of the formula:

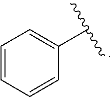

In certain particular embodiments, R₂ is of the formula:

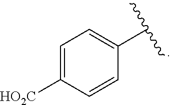

In certain particular embodiments, R₂ is a substituted or unsubstituted phenyl moiety. In certain embodiments, R₂ is substituted phenyl moiety (e.g., a phenyl ring with 1, 2, 3, 4, or 5 substituents). In other embodiments, R₂ is a substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain embodiments, R_{B} is

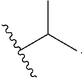

In other embodiments, R_{B} is t-butyl. In certain particular embodiments, R₂ is —CO₂R_{B}, wherein R_{B} is one of the formulae:

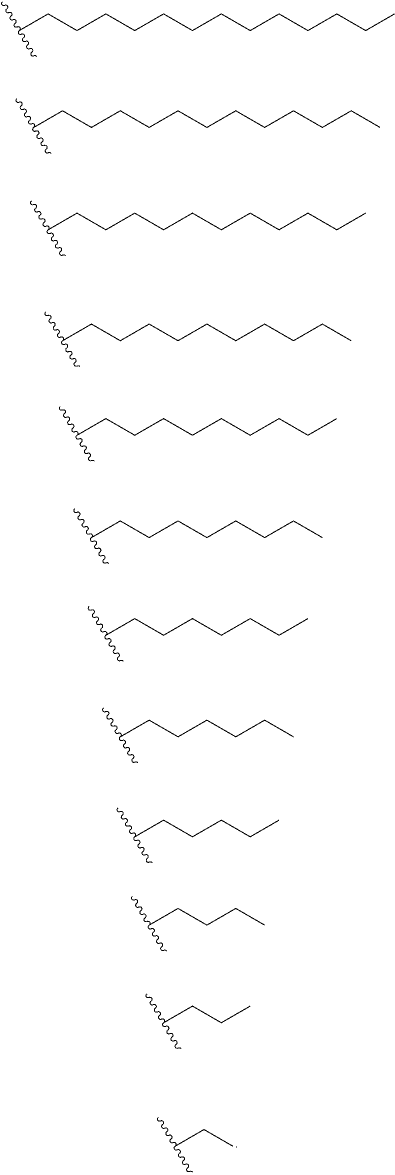

As would be appreciated by one of skill in this art, any of the above alkyl groups may be substituted, branched, unsaturated, and/or cyclic. In certain particular embodiments, R₂ is —CO₂R_{B}, wherein R_{B} is aryl or arylalkyl. In certain particular embodiments, R₂ is —CO₂R_{B}, wherein R_{B} is of the formula:

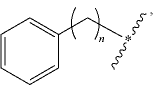

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

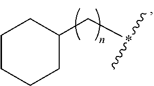

wherein n is an integer between 0 and 12, inclusive. In certain embodiments, n is 0. In certain embodiments, n is 1. In other embodiments, n is 2, 3, 4, 5, or 6.

In other embodiments, R₃ is substituted or unsubstituted, branched or unbranched aliphatic. In yet other embodiments, R₃ is C₁-C₆ alkyl. In certain particular embodiments, R₃ is methyl.

In certain embodiments, R₃ is of one of the formulae:

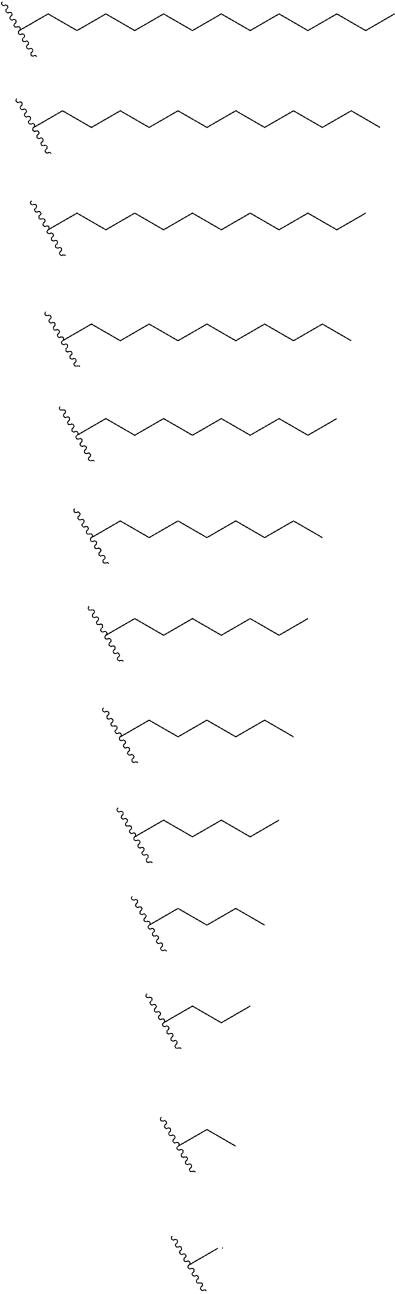

As would be appreciated by one of skill in this art, any of the above alkyl groups may be substituted, branched, unsaturated, and/or cyclic. In yet other embodiments, R₃ is a substituted or unsubstituted, branched or unbranched heteroaliphatic moiety. In still other embodiments, R₃ is a substituted or unsubstituted acyl moiety. In other embodiments, R₃ is a substituted or unsubstituted aryl moiety. In certain particular embodiments, R₃ is of the formula:

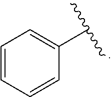

In certain particular embodiments, R₃ is of the formula:

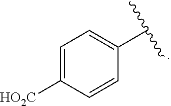

In certain particular embodiments, R₃ is a substituted or unsubstituted phenyl moiety. In certain embodiments, R₃ is a substituted phenyl (e.g., a phenyl ring with 1, 2, 3, 4, or 5 substituents). In other embodiments, R₃ is a substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain embodiments, R_{C} is

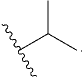

In other embodiments, R_{C} is t-butyl. In certain particular embodiments, R₃ is —CO₂R_{C}, wherein R_{C} is one of the formulae:

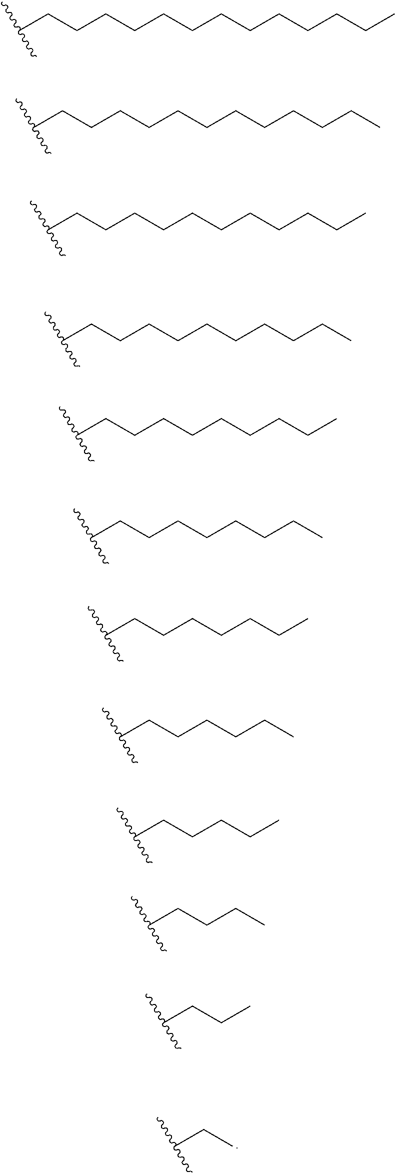

As would be appreciated by one of skill in this art, any of the above alkyl groups may be substituted, branched, unsaturated, and/or cyclic. In certain particular embodiments, R₃ is —CO₂R_{C}, wherein R_{C} is aryl or arylalkyl. In certain particular embodiments, R₃ is —CO₂R_{C}, wherein R_{C} is of the formula:

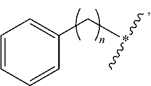

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

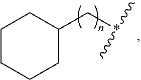

wherein n is an integer between 0 and 12, inclusive. In certain embodiments, n is 0. In certain embodiments, n is 1. In other embodiments, n is 2, 3, 4, 5, or 6.

In other embodiments, R₄ is substituted or unsubstituted, branched or unbranched aliphatic. In yet other embodiments, R₄ is C₁-C₆ alkyl. In certain particular embodiments, R₄ is methyl.

In certain embodiments, R₄ is of one of the formulae:

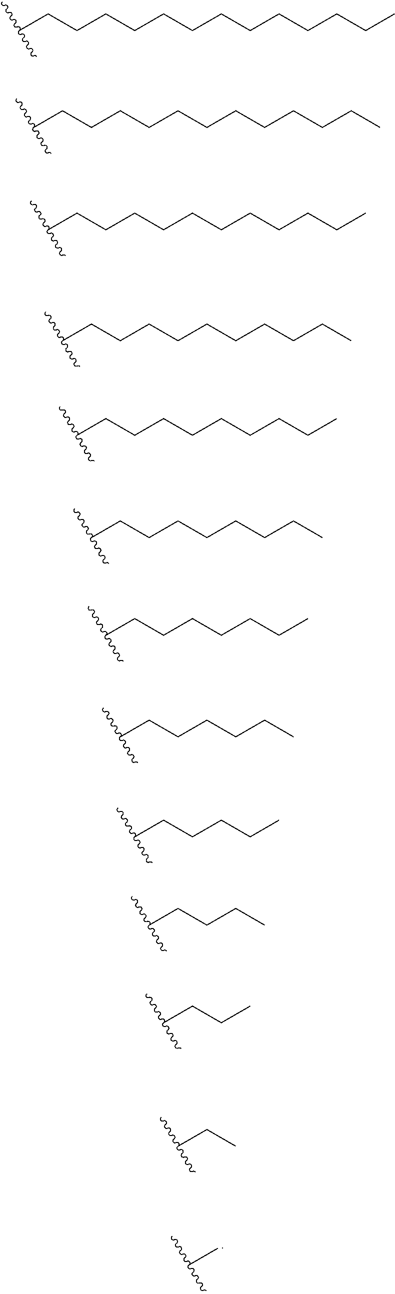

As would be appreciated by one of skill in this art, any of the above alkyl groups may be substituted, branched, unsaturated, and/or cyclic. In yet other embodiments, R₄ is a substituted or unsubstituted, branched or unbranched heteroaliphatic moiety. In still other embodiments, R₄ is a substituted or unsubstituted acyl moiety. In other embodiments, R₄ is a substituted or unsubstituted aryl moiety. In certain particular embodiments, R₄ is of the formula:

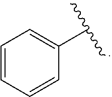

In certain particular embodiments, R₄ is of the formula:

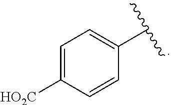

In certain particular embodiments, R₄ is a substituted or unsubstituted phenyl moiety. In certain embodiments, R₄ is substituted phenyl moiety (e.g., a phenyl ring with 1, 2, 3, 4, or 5 substituents). In other embodiments, R₄ is a substituted or unsubstituted heteroaryl moiety. In certain embodiments, R₄ is —C(═O)R_{D}. In other embodiments, R₄ is —CO₂R_{D}. In certain embodiments, R_{D} is C₁-C₆ alkyl. In certain particular embodiments, R_{D} is methyl. In certain embodiments, R_{D}

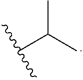

In other embodiments, R_{D} is t-butyl. In certain particular embodiments, R₄ is —CO₂R_{D}, wherein R_{D} is one of the formulae:

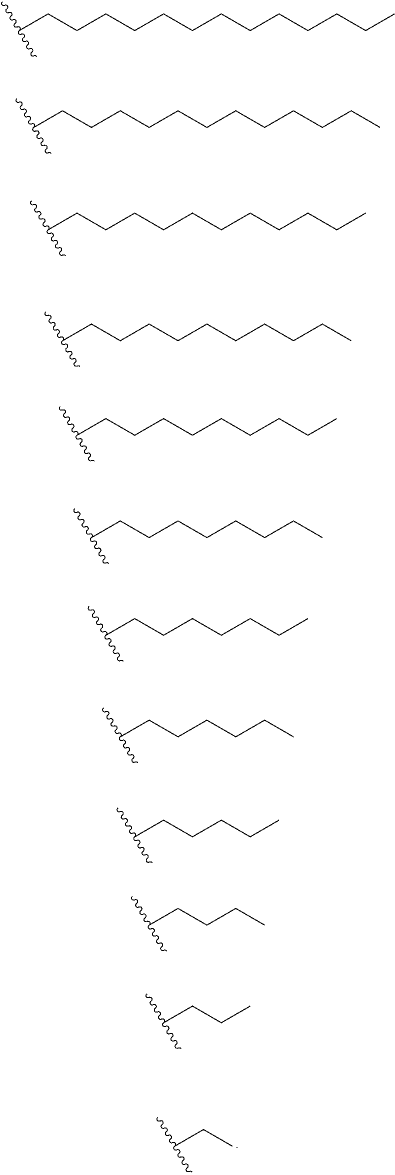

As would be appreciated by one of skill in this art, any of the above alkyl group may be substituted, branched, unsaturated, and/or cyclic. In certain particular embodiments, R₄ is —CO₂R_{D}, wherein R_{D} is aryl or arylalkyl. In certain particular embodiments, R₄ is —CO₂R_{D}, wherein R_{D} is of the formula:

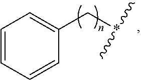

wherein n is an integer between 0 and 12, inclusive. In certain embodiments, n is 0. In certain embodiments, n is 1. In other embodiments, n is 2, 3, 4, 5, or 6. In certain particular embodiments, R₄ is —CO₂R_{D}, wherein R_{D} is of the formula:

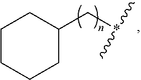

wherein n is an integer between 0 and 12, inclusive. In certain embodiments, n is 0. In certain embodiments, n is 1. In other embodiments, n is 2, 3, 4, 5, or 6.

In other embodiments, R₁ is —CO₂R_{A}, and R₂ and R₃ are both methyl. In certain embodiments, at least one of R₁, R₂, R₃, and R₄ is fluorine.

Exemplary tetrasubstituted fluorinated monomers include:

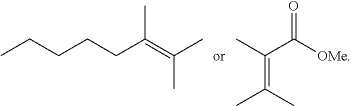

In certain embodiments, the monomer is a diacrylate or dimethacrylate. In certain embodiments, the fluorinated diacrylate is of the formula:

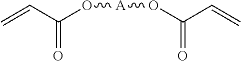

wherein A is a linker. In certain embodiments, the diacrylate is of the formula:

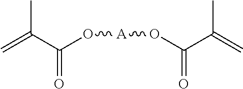

wherein A is a linker. In certain embodiments, A is a substituted or unsubstituted, branched or unbranched, cyclic or acyclic aliphatic; substituted or unsubstituted, branched or unbranched, cyclic or acyclic heteroaliphatic; substituted or unsubstituted aryl; or substituted or unsubstituted heteroaryl. In certain embodiments, the linker A is an alkyl linker. In certain embodiments, the linker A is of one of the formulae:

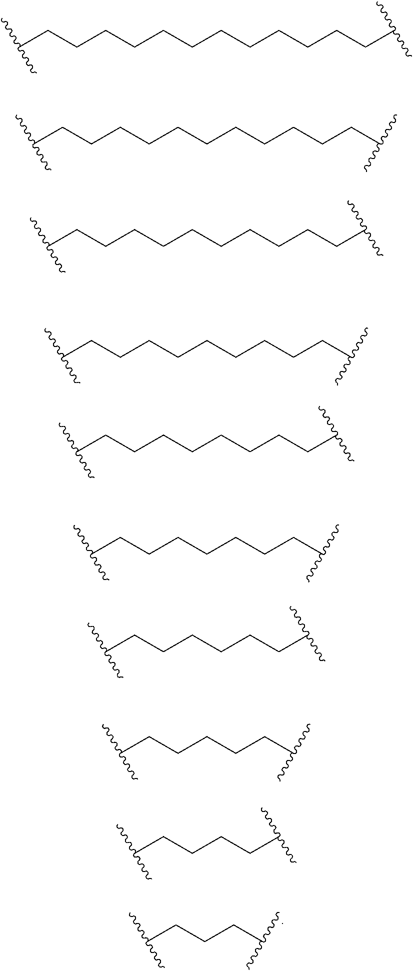

As would be appreciated by one of skill in this art, any of the above alkyl groups may be substituted, branched, unsaturated, and/or cyclic. In other embodiments, the linker A is of one of the formulae:

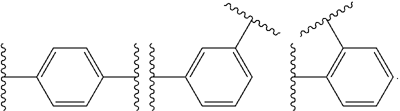

Exemplary diacrylate and dimethacrylates include:

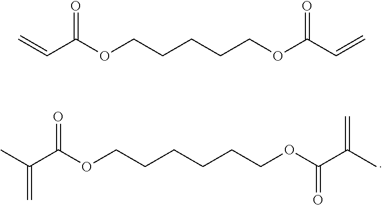

In certain embodiments, the monomer is a triacrylate or trimethacrylate. In certain embodiments, the monomer is of the formula:

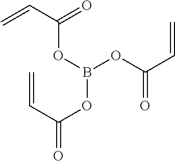

wherein B is a linker.

In other embodiments, the monomer is of the formula:

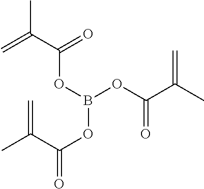

wherein B is a linker.

In certain embodiments, linker B is a substituted or unsubstituted, branched or unbranched, cyclic or acyclic aliphatic; substituted or unsubstituted, branched or unbranched, cyclic or acyclic heteroaliphatic; substituted or unsubstituted aryl; or substituted or unsubstituted heteroaryl. In certain embodiments, the linker B is a branched, alkyl linker. In certain embodiments, the linker B is a aryl linker. In certain embodiments, the linker B is of the formula:

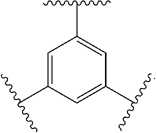

An examplary trimethacrylate is of the formula:

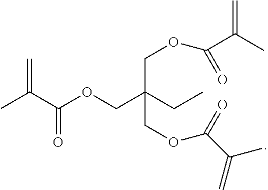

In certain embodiments, the fluorinated monomer is a tetraacrylate or tetramethacrylate. Tetraacrylates may be prepared by reacting diacrylates or dimethacrylates with a diamine. An exemplary tetramethacrylate is of the formula:

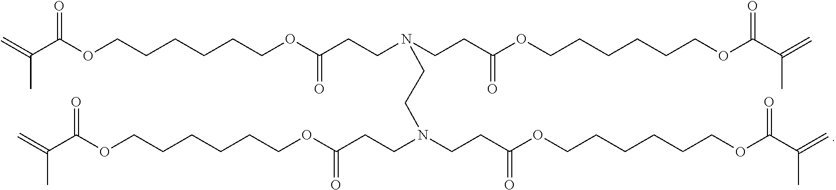

In other embodiments, the monomer is a pentaacrylate or pentamethacrylate. In still other embodiments, the monomer is an even higher acrylate or methacrylate.

In certain embodiments, the monomer is an alkyne. In certain embodiments, the alkynyl monomer is of the formula:

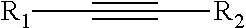

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, R₁ is hydrogen. In other embodiments, R₁ is a substituted or unsubstituted, branched or unbranched aliphatic moiety. In certain embodiments, R₁ is of one of the formulae:

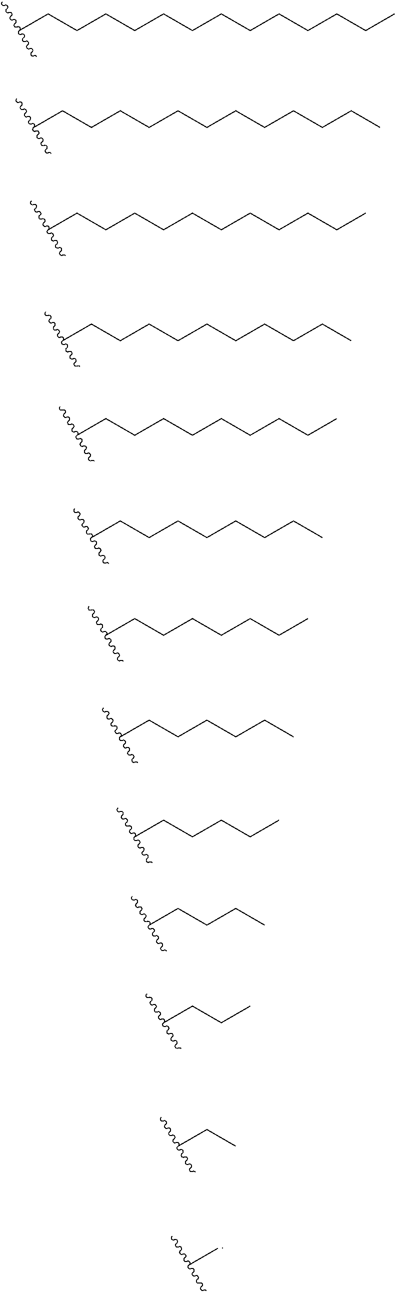

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

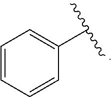

In certain particular embodiments, R₁ is of the formula:

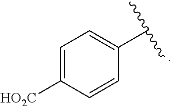

In certain particular embodiments, R₁ is a substituted or unsubstituted phenyl moiety. In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl.

In certain particular embodiments, R_{A} is methyl. In certain embodiments, R_{A} is

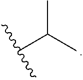

In other embodiments, R_{A} is t-butyl. In certain particular embodiments, R₁ is —CO₂R_{A}, wherein R_{A} is one of the formulae:

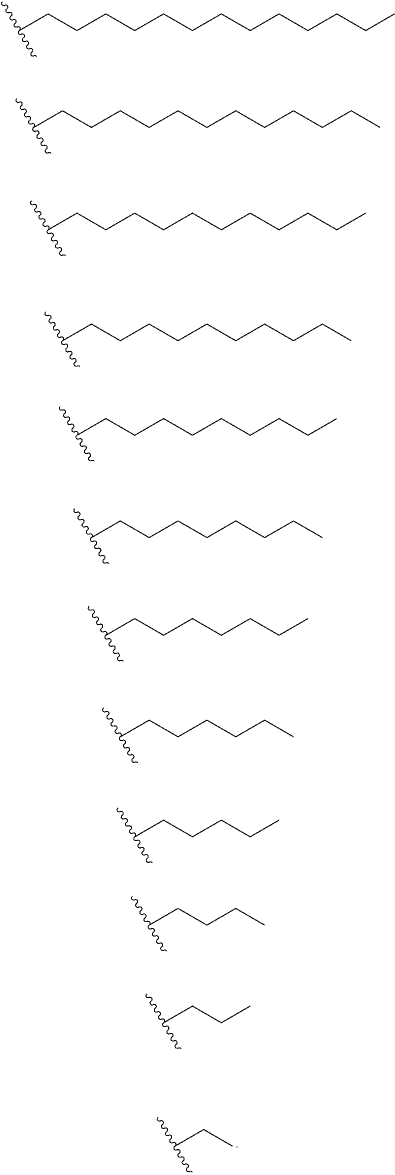

As would be appreciated by one of skill in this art, any of the above alkyl group may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

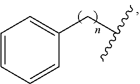

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

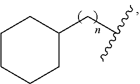

wherein n is an integer between 0 and 12, inclusive. In certain embodiments, n is 0. In certain embodiments, n is 1. In other embodiments, n is 2, 3, 4, 5, or 6.

In other embodiments, R₂ is hydrogen. In other embodiments, R₂ is substituted or unsubstituted, branched or unbranched aliphatic. In certain embodiments, R₂ is an alkyl moiety. In yet other embodiments, R₂ is C₁-C₆ alkyl. In certain particular embodiments, R₂ is methyl. In certain embodiments, R₂ is a aryl or heteroaryl moiety. In certain embodiments, R₂ is a phenyl moiety.

In certain embodiments, R₁ is —CO₂R_{A}. In certain embodiments, R₁ is —CO₂R_{A}, and R₂ is hydrogen. In other embodiments, R₁ is —CO₂R_{A}, and R₂ is methyl.

Exemplary fluorinated alkynyl monomers include:

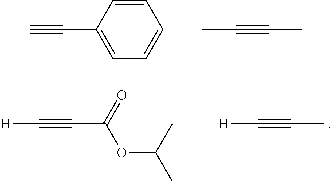

In certain other embodiments, the monomer is an oligomer. The monomers described herein are partially polymerized to form oligomers. The oligomers are applied to hair and further polymerized on the treated hair. In certain embodiments, the oligomers are of a molecular weight sufficient to apply the oligomer to hair. In certain embodiments, the molecular weight of the oligomer is less than 1,000 g/mol. In certain embodiments, the molecular weight is less than 1,500 g/mol. In other embodiments, the molecular weight is less than 2,000 g/mol. In other embodiments, the molecular weight is less than 3,000 g/mol. In other embodiments, the molecular weight is less than 4,000 g/mol. In yet other embodiments, the molecular weight is less than 5,000 g/mol.

In certain embodiments, the monomer is mixed with one or more different monomers. The resulting polymer is a co-polymer. As would be appreciated by those of skill in this art, a co-polymer may have desirable properties not attainable with a polymer resulting from the polymerization of one monomer alone. In certain embodiments, two different monomers are applied to hair. In other embodiments, three different monomers are applied to hair. When different monomer are used, the monomers are applied to hair simultaneously or separately. In certain embodiments, the monomers are all in the same solution which is applied to the hair.

Exemplary monomers useful in accordance with the present invention include trimethylolpropane trimethacrylate; 1,3-bis(3-methacryloyloxypropyl)-1,1,3,3-tetramethyldisiloxane; 1,3-butanediol dimethacrylate; 1,4-butanediol dimethacrylate; 1,6-hexanediol dimethacrylate; bisphenol A dimethacrylate; bisphenol A ethoxylate dimethacrylate; bisphenol A glycerolate dimethacrylate; di(ethylene glycol) dimethacrylate; diurethane dimethacrylate, mixture of isomers; ethylene glycol dimethacrylate; glycerol dimethacrylate, mixture of isomers; neopentyl glycol dimethacrylate; poly(ethylene glycol) dimethacrylate; poly(lauryl methacrylate-co-ethylene glycol dimethacrylate); poly(methyl methacrylate-co-ethylene glycol dimethacrylate); poly(propylene glycol) dimethacrylate; tetraethylene glycol dimethacrylate; triethylene glycol dimethacrylate; 1,1,1,3,3,3-hexafluoroisopropyl methacrylate; 2-(9H-carbazol-9-yl)ethyl acrylate; 2-(diethylamino)ethyl methacrylate; 2-(dimethylamino)ethyl methacrylate; 2-(methacryloyloxy)ethyl acetoacetate; 2-(methylthio)ethyl methacrylate; 2-(tert-butylamino)ethyl methacrylate; 2-(trimethylsilyloxy)ethyl methacrylate; 2,2,2-trifluoroethyl methacrylate; 2,2,3,3,3-pentafluoropropyl methacrylate; 2,2,3,3,4,4,4-heptafluorobutyl methacrylate; 2,2,3,3,4,4,5,5-octafluoropentyl methacrylate; 2,2,3,4,4,4-hexafluorobutyl methacrylate; 2-[3-(2H-benzotriazol-2-yl)-4-hydroxyphenyl]ethyl methacrylate; 2-aminoethyl methacrylate hydrochloride; 2-butoxyethyl methacrylate; 2-ethoxyethyl methacrylate; 2-ethylhexyl methacrylate; 2-hydroxyethyl methacrylate; 2-methyl-2-nitropropyl methacrylate; 2-naphthyl methacrylate; 3-(acryloyloxy)-2-hydroxypropyl methacrylate; 3-(diethoxymethylsilyl)propyl methacrylate; 3-(dimethylchlorosilyl)propyl methacrylate; 3-(trichlorosilyl)propyl methacrylate; 3-(dimethylchlorosilyl)propyl methacrylate; 3-(trichlorosilyl)propyl methacrylate; 3-(trimethoxysilyl)propyl methacrylate; 3,3,4,4,5,5,6,6,6,-nonafluorohexyl methacrylate; 3,3,4,4,5,5,6,6,7,7,8,8,8-tridecafluorooctyl methacrylate; 3,3,4,4,5,5,6,6,7,7,8,8,9,10,10,10-hexadecafluoro-9-trifluoromethyl)decyl methacrylate; 3,3,4,4,5,5,6,6,7,7,8,8,9,9,10,10,10-heptadecafluorodecyl methacrylate; 3,3,4,4,5,5,6,6,7,7,8,8,9,9,10,10,11,11,12,12,12-heneicosafluorododecyl methacrylate; 3,3,4,4,5,5,6,6,7,7,8,8,9,9,10,10,11,12,12,12-eicosafluoro-11-(trifluoromethyl)dodecyl methacrylate; 3,3,4,4,5,5,6,6,7,8,8,8-dodecafluoro-7-(trifluoromethyl)octyl methacrylate; 3,3,4,4,5,6,6,6-octafluoro-5-(trifluoromethyl)hexyl methacrylate; 3,3,5-trimethylcyclohexyl methacrylate, mixture of isomers; 3-[(3,5,7,9,11,13,15-heptacyclopentylpentacyclo[9.5.1.1^{3,9}.1^{5,15}.1^{7,13}]octasiloxan-1-yloxy)dimethylsilyl]propyl methacrylate; 3-[tris(trimethylsiloxy)silyl]propyl methacrylate; 3-chloro-2-hydroxypropyl methacrylate; 3-sulfopropyl methacrylate; 4,4,5,5,6,6,7,7,8,8,9,9,10,11,11,11-hexadecafluoro-2-hydroxy-10-(trifluoromethyl)undecyl methacrylate; 4,4,5,5,6,6,7,7,8,9,9,9-dodecafluoro-2-hydroxy-8-(trifluoromethyl)nonyl methacrylate; 4,4,5,5,6,7,7,7-octafluoro-2-hydroxy-6-(trifluoromethyl)heptyl methacrylate; 6-[4-(4-cyanophenyl)phenoxy]hexyl methacrylate; 9-anthracenylmethyl methacrylate; 9H-carbazole-9-ethylmethacrylate; allyl methacrylate; benzyl methacrylate; butyl methacrylate; cyclohexyl methacrylate; decyl methacrylate; di(ethylene glycol) ethyl ether methacrylate; di(ethylene glycol) methyl ether methacrylate; di(propylene glycol) allyl ether methacrylate, mixture of isomers; Disperse Red 1 methacrylate; Disperse Red 13 methacrylate; Disperse yellow 7 methacrylate; ethyl methacrylate; ethylene glycol dicyclopentenyl ether methacrylate; ethylene glycol methyl ether methacrylate; ethylene glycol phenyl ether methacrylate; furfuryl methacrylate; glycidyl methacrylate; glycol methacrylate; glycosyloxyethyl methacrylate; hexyl methacrylate; hydroxybutyl methacrylate, mixture of isomers; hydroxypropyl methacrylate; isobornyl methacrylate; isobutyl methacrylate; isodecyl methacrylate; lauryl methacrylate; methyl methacrylate; stearyl methacrylate; tert-butyl methacrylate; tetrahydrofurfuryl methacrylate; tridecyl methacrylate; trimethylsilyl methacrylate; vinyl methacrylate; glycerol propoxylate (1PO/OH) triacrylate; pentaerythritol triacrylate; trimethylolpropane ethoxylate triacrylate; trimethylolpropane propoxylate triacrylate; trimethylolpropane triacrylate; di(trimethylolpropane) tetraacrylate; pentaerythritol tetraacrylate; dipentaerythritol pentaacrylate; ethoxylated pentaerythritol tetraacrylate; low viscosity dipentaerythritol pentaacrylate; pentaacrylate ester; pentaerythritol tetraacrylate; trimethylolpropane triacrylate; ethoxylated trimethylolpropane triacrylate; propoxylated glycerol triacrylate; pentaerythritol triacrylate; propoxylated glyceryl triacrylate; propoxylated trimethylolpropane triacrylate; trimethylolpropane trimethacrylate; tris (2-hydroxy ethyl)isocyanurate triacrylate; tris (2-hydroxy ethyl)isocyanurate triacrylate; polybutadiene diacrylate; and polybutadiene dimethacrylate. In certain particular embodiments, monomer is ethyl acrylate; vinyl acrylate; 1,3-butanediol diacrylate; dipentaerythritol pentaacrylate; tridecyl methacrylate; styrene; and 3,4-epoxycyclohexylmethyl 3′,4′-epoxycyclohexane carboxylate. In certain embodiments, the monomer is a polybutadiene di(meth)acrylate oligomer. In certain embodiments, the monomer is tricyclodecane dimethanol diacrylate. In certain embodiments, the monomer is tricyclodecane dimethanol dimethacrylate.

In certain embodiments, a fluorinated monomer is polymerized on hair based on the inventive hair treatment system. The fluorinated monomer comprises a functional group suitable for polymerization and at least one fluorine atom. Any functional group that can be polymerized using a free radical or ionic polymerization reaction can be used. Certain such functional groups are described. Typically, the functional group includes a degree of unsaturation (e.g., a double bond or triple bond). Exemplary functional groups suitable for polymerization include alkenes, alkynes, carbonyls, imines, thiocarbonyls, acrylates, methacrylates, acrylates, crotonates, styrenes, nitriles, cyano, vinyl, styrene, crotonate, cinnamate, dienes, trienes, eneynes, maleimides, etc.

The fluorinated monomer may range from including one fluorine atom to being perfluorinated. In certain embodiments, a functional group of the monomer is perfluorinated such as, for example, an alkyl, alkenyl, alkynyl, acyl, aryl, heteroaryl, heterocyclic, or carbocyclic moiety. In certain embodiments, the fluorinated monomer includes at least 1, 2, 3, 4, 5, 6, 7, 8, 9, 10, 11, 12, 13, 14, 15, 16, 17, 18, 19, or 20 fluorine atoms. In other embodiments, the fluorinated monomer contains at least 10, at least 15, at least 20, at least 25, at least 30, or at least 40 fluorine atoms. As would be appreciated by one of skill in this art, the larger the monomer the more fluorine atoms the monomer is likely to have. Furthermore, the monomer should include enough fluorine atoms so that the resulting polymer imparts the desired characteristics when polymerized on hair (e.g., look, feel).

In certain embodiments, at least 10%, 20%, 30%, 40%, 50%, 60%, 70%, 80%, 90%, 95%, or 99% of the total number of hydrogen and fluorine atoms are fluorine atoms in the fluorinated monomer. In certain embodiments, at least 50% of the total number of hydrogen and fluorine atoms are fluorine atoms in the fluorinated monomer. In certain embodiments, at least 80% of the total number of hydrogen and fluorine atoms are fluorine atoms in the fluorinated monomer. In certain embodiments, at least 90% of the total number of hydrogen and fluorine atoms are fluorine atoms in the fluorinated monomer. In certain embodiments, the fluorinated monomer is perfluorinated (i.e., all hydrogen atoms, or at least all non-exchangeable hydrogen atoms, are replaced with fluorine atoms).

In certain embodiments, the fluorinated monomer is a fluorinated alkene. In certain particular embodiments, the fluorinated alkene is monosubstituted. In other embodiments, the fluorinated alkene is disubstituted. Disubstituted fluorinated alkene may be either in the cis or trans configuration or a mixture thereof. In yet other embodiments, the fluorinated alkene is trisubstituted. The trisubstituted fluorinated alkene may be in either the E or Z configuration or a mixture thereof. In still other embodiments, the fluorinated alkene is tetrasubstituted. Again, various isomers are possible and are considered part of this invention. In certain embodiments, the fluorinated monomer is a fluorinated alkyne.

In certain embodiments, the monosubstituted fluorinated monomer is of the formula:

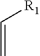

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and wherein R₁ comprises at least one fluorine atom.

In certain embodiments, R₁ contains more than 1, 2, 3, 4, 5, 10, 15, 20, or 25 fluorine atoms. In certain embodiments, R₁ is fluorine. In other embodiments, R₁ is a fluorinated, substituted or unsubstituted, branched or unbranched aliphatic moiety. In certain embodiments, R₁ is a fluorinated alkyl moiety. In certain embodiments, R₁ is of one of the formulae:

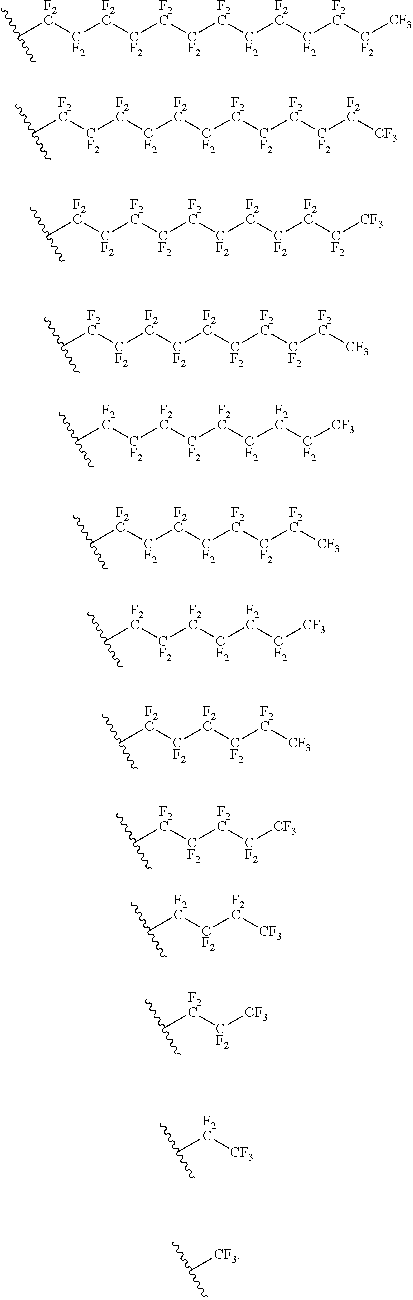

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

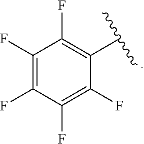

In certain particular embodiments, R₁ is of the formula:

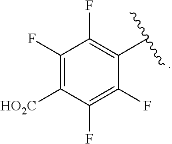

In certain particular embodiments, R₁ is a fluorinated, substituted or unsubstituted phenyl moiety. In certain embodiments, R₁ is fluorinated phenyl (e.g., a phenyl ring with 1, 2, 3, 4, or 5 fluorine substituents). In other embodiments, R₁ is a fluorinated, substituted or unsubstituted heteroaryl moiety. In certain embodiments, R₁ is —C(═O)R_{A}, wherein R_{A} comprises at least one fluorine atom. In other embodiments, R₁ is —CO₂R_{A}, wherein R_{A} comprises at least one fluorine atom. In certain particular embodiments, R₁ is —CO₂R_{A}, wherein R_{A} is one of the formulae:

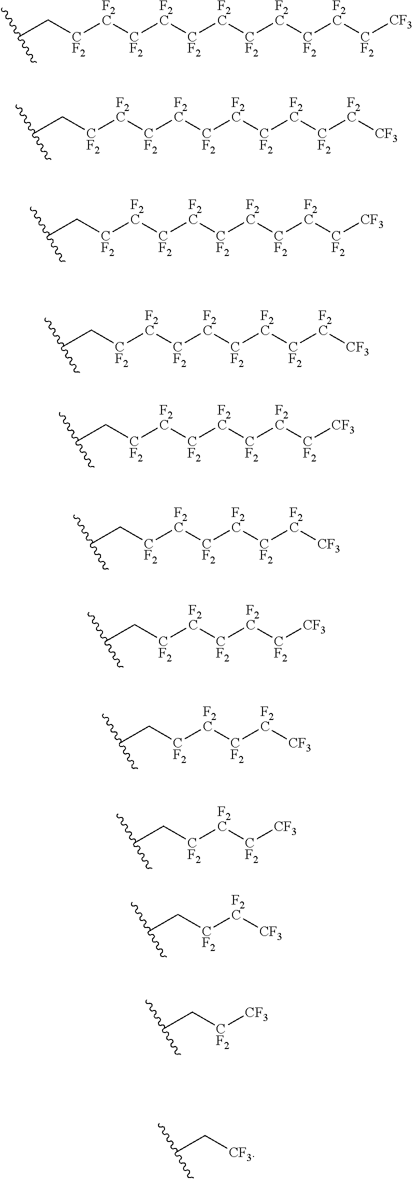

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

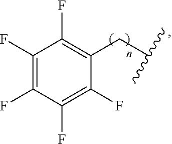

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

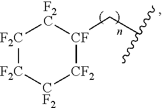

wherein n is an integer between 0 and 12, inclusive. In certain embodiments, n is 0. In certain embodiments, n is 1. In other embodiments, n is 2, 3, 4, 5, or 6.

In certain embodiments, the fluorinated monomer is a fluorinated acrylate of formula:

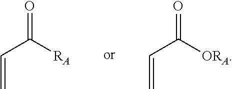

Exemplary monosubstituted fluorinated monomers include:

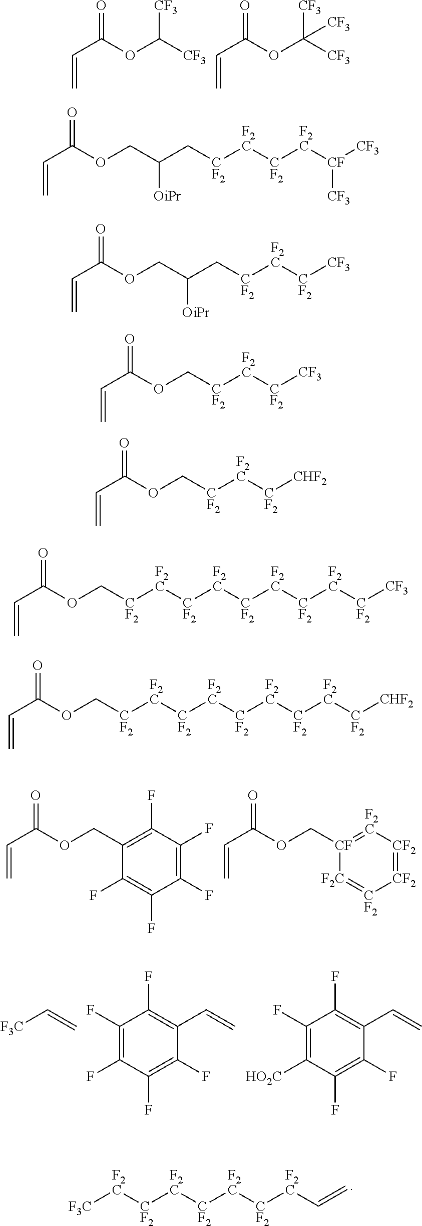

In certain embodiments, the disubstituted fluorinated monomer is of one of the formulae:

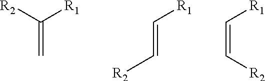

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

wherein R₁ or R₂ comprises at least one fluorine atom.

In certain embodiments, R₁ contains more than 1, 2, 3, 4, 5, 10, 15, 20, or 25 fluorine atoms. In certain embodiments, R₁ is fluorine. In other embodiments, R₁ is a fluorinated, substituted or unsubstituted, branched or unbranched aliphatic moiety. In certain embodiments, R₁ is a fluorinated alkyl moiety. In certain embodiments, R₁ is of one of the formulae:

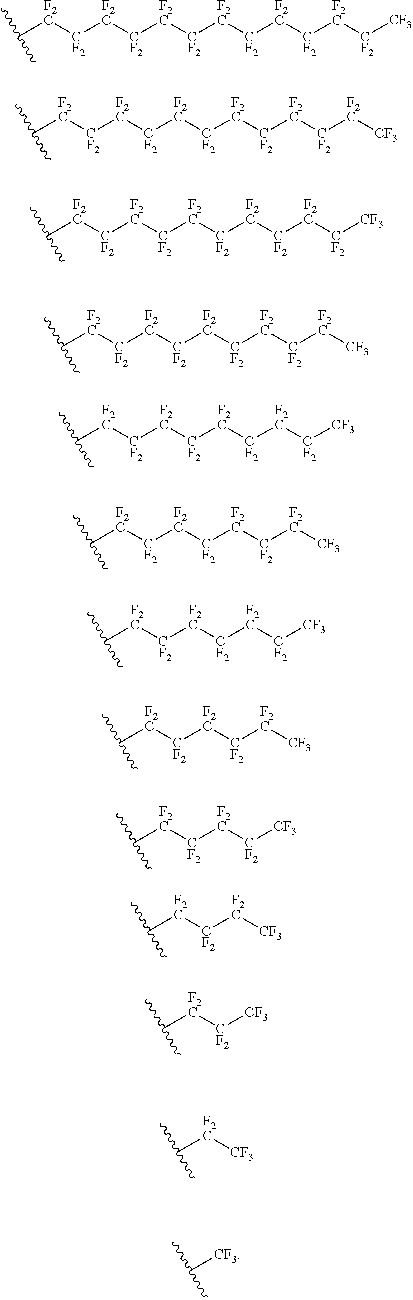

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

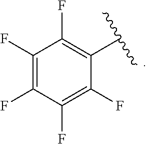

In certain particular embodiments, R₁ is of the formula:

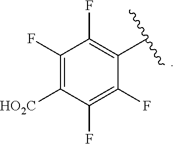

In certain particular embodiments, R₁ is a fluorinated, substituted or unsubstituted phenyl moiety. In certain embodiments, R₁ is fluorinated phenyl (e.g., a phenyl ring with 1, 2, 3, 4, or 5 fluorine substituents). In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

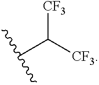

In other embodiments, R_{A} is t-butyl. In certain particular embodiments, R₁ is —CO₂R_{A}, wherein R_{A} is one of the formulae:

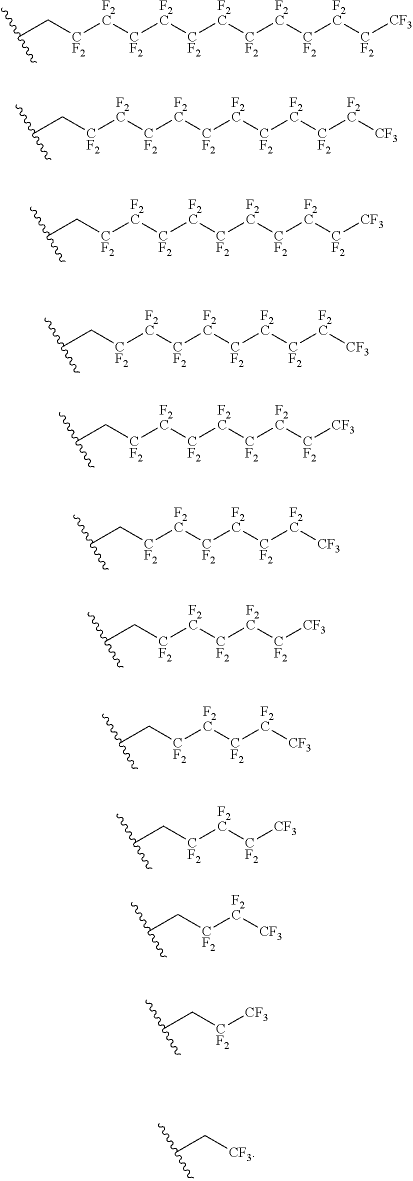

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

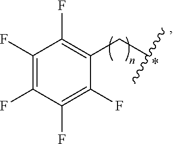

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

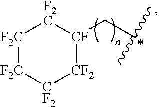

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, only one of R₁ and R₂ includes fluorine atoms. In other embodiments, both R₁ and R₂ include fluorine atoms. In certain embodiments, R₂ is fluorine. In other embodiments, R₂ is substituted or unsubstituted, branched or unbranched aliphatic. In yet other embodiments, R₂ is C₁-C₆ alkyl. In certain embodiments, R₂ is a perfluorinated alkyl moiety. In certain particular embodiments, R₂ is methyl. In certain embodiments R₂ is —CF₃, —CHF₂, or —CH₂F. In certain embodiments, R₂ is a fluorine-substituted aryl or heteroaryl moiety. In certain embodiments, R₂ is a fluorine-substituted phenyl moiety. In certain particular embodiments, R₂ is a perfluorinated phenyl moiety.

In certain embodiments, R₁ is —CO₂R_{A}, and R₂ is fluorine. In other embodiments, R₁ is —CO₂R_{A}, and R₂ is C₁-C₆ alkyl, optionally substituted with fluorine. In other embodiments, R₁ is —CO₂R_{A}, and R₂ is methyl. In yet other embodiments, R₁ is —CO₂R_{A}, and R₂ is —CF₃.

In certain embodiments, the fluorinated monomer is a fluorinated methacrylate of formula:

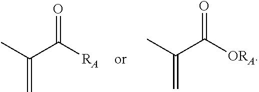

In certain embodiments, the fluorinated monomer is a fluorinated acrylate of formula:

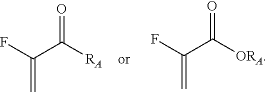

In certain embodiments, the fluorinated monomer is a fluorinated methacrylate of formula:

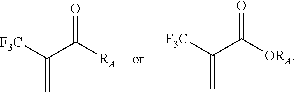

In certain embodiments, the fluorinated monomer is a fluorinated crotonate of formula:

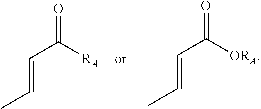

In certain embodiments, the fluorinated monomer is a fluorinated crontonate of formula:

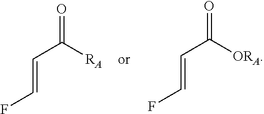

In certain embodiments, the fluorinated monomer is a fluorinated crotonate of formula:

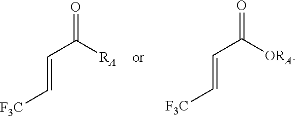

Exemplary disubstituted fluorinated monomers include:

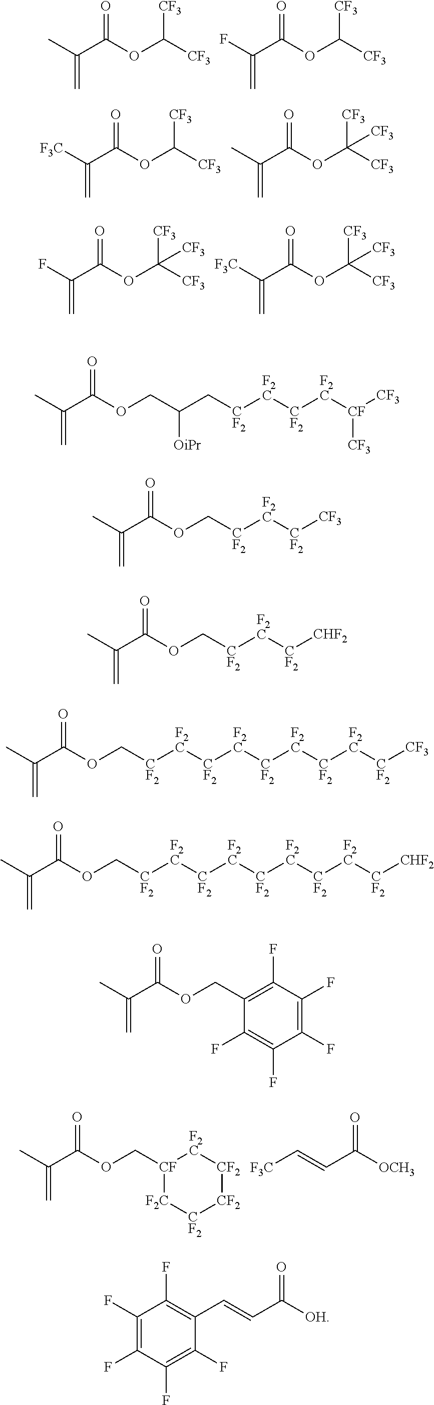

In certain embodiments, the trisubstituted fluorinated monomer is of one of the formulae:

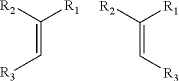

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

wherein R₁, R₂, or R₃ comprises at least one fluorine atom.

In certain embodiments, R₁ contains more than 1, 2, 3, 4, 5, 10, 15, 20, or 25 fluorine atoms. In certain embodiments, R₁ is fluorine. In other embodiments, R₁ is a fluorinated, substituted or unsubstituted, branched or unbranched aliphatic moiety. In certain embodiments, R₁ is a fluorinated alkyl moiety. In certain embodiments, R₁ is of one of the formulae:

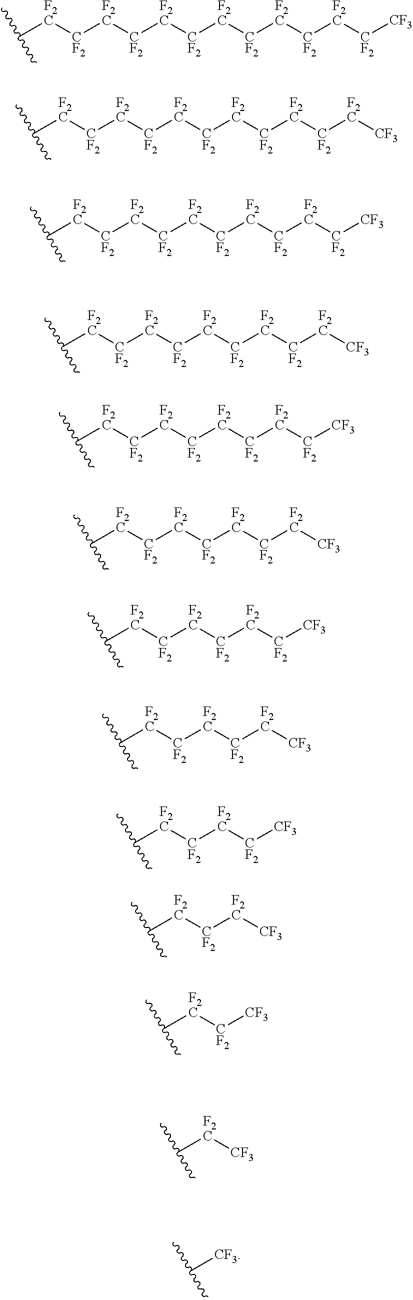

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

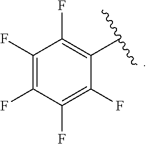

In certain particular embodiments, R₁ is of the formula:

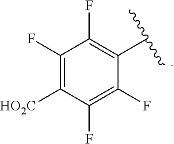

In certain particular embodiments, R₁ is a fluorinated, substituted or unsubstituted phenyl moiety. In certain embodiments, R₁ is fluorinated phenyl (e.g., a phenyl ring with 1, 2, 3, 4, or 5 fluorine substituents). In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

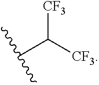

In other embodiments, R_{A} is t-butyl. In certain particular embodiments, R₁ is —CO₂R_{A}, wherein R_{A} is one of the formulae:

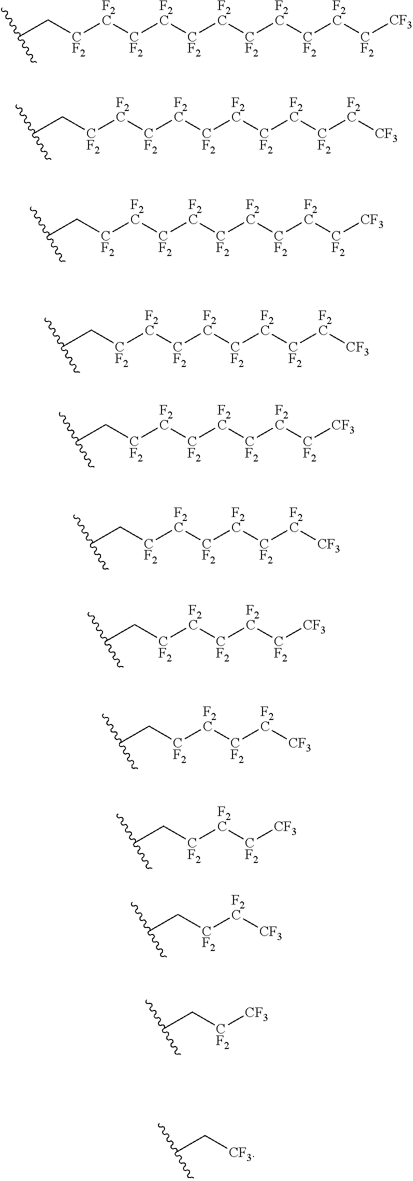

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

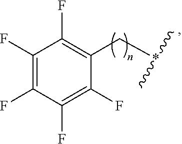

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

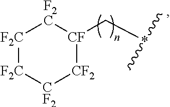

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, R₂ is fluorine. In other embodiments, R₂ is substituted or unsubstituted, branched or unbranched aliphatic. In yet other embodiments, R₂ is C₁-C₆ alkyl, optionally substituted with a fluorine. In certain embodiments, R₂ is a perfluorinated C₁-C₆ alkyl moiety. In certain particular embodiments, R₂ is methyl. In certain embodiments R₂ is —CF₃, —CHF₂, or —CH₂F.

In certain embodiments, R₂ is of one of the formulae:

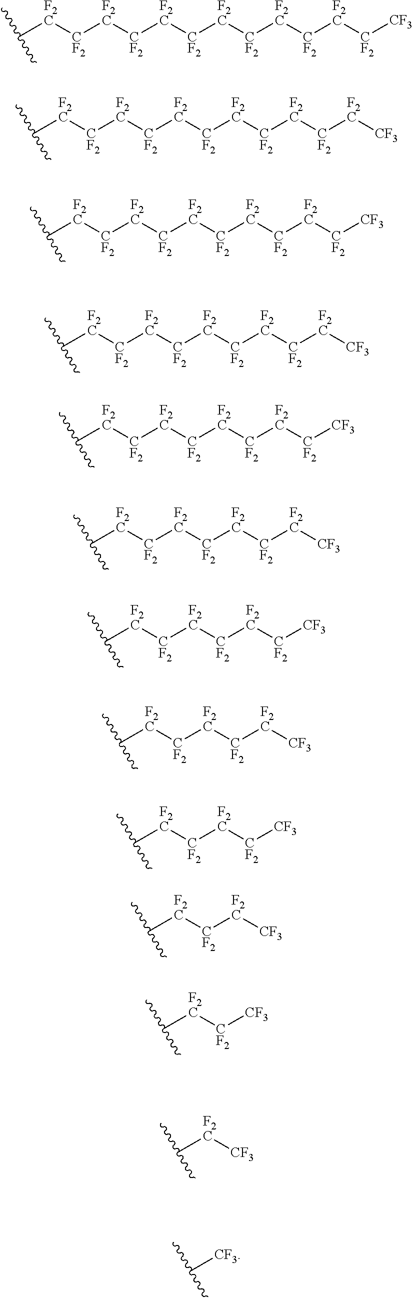

As would be appreciated by one of skill in this art, any of the above perfluorinated alkyl groups may be partially fluorinated, substituted, branched, unsaturated, and/or cyclic. In yet other embodiments, R₂ is a fluorinated, substituted or unsubstituted, branched or unbranched heteroaliphatic moiety. In still other embodiments, R₂ is a fluorinated, substituted or unsubstituted acyl moiety. In other embodiments, R₂ is a fluorinated, substituted or unsubstituted aryl moiety. In certain particular embodiments, R₂ is of the formula:

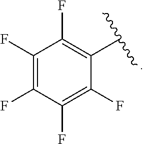

In certain particular embodiments, R₂ is of the formula:

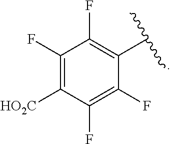

In certain particular embodiments, R₂ is a fluorinated, substituted or unsubstituted phenyl moiety. In certain embodiments, R₂ is fluorinated phenyl (e.g., a phenyl ring with 1, 2, 3, 4, or 5 fluorine substituents). In other embodiments, R₂ is a fluorinated, substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain particular embodiments, R_{B} is —CF₃. In certain embodiments, R_{B} is

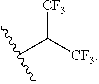

In other embodiments, R_{B} is t-butyl. In certain particular embodiments, R₂ is —CO₂R_{B}, wherein R_{B} is one of the formulae:

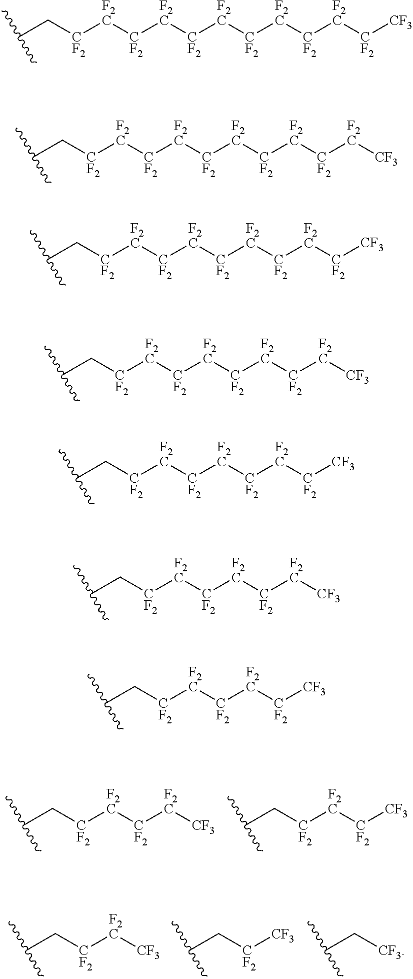

As would be appreciated by one of skill in this art, any of the above fluorinated alkyl groups may be partially fluorinated, substituted, branched, unsaturated, and/or cyclic. In certain particular embodiments, R₂ is —CO₂R_{B}, wherein R_{B} is fluorinated aryl or fluorinated arylalkyl. In certain particular embodiments, R₂ is —CO₂R_{B}, wherein R_{B} is of the formula:

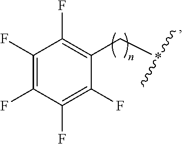

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

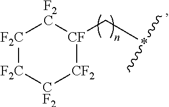

wherein n is an integer between 0 and 12, inclusive. In certain embodiments, n is 0. In certain embodiments, n is 1. In other embodiments, n is 2, 3, 4, 5, or 6.

In certain embodiments, R₃ includes more than 1, 2, 3, 4, 5, 10, 15, 20, or 25 fluorine atoms. In certain embodiments, only one of R₁, R₂, and R₃ includes fluorine atoms. In certain other embodiments, only two of R₁, R₂, and R₃ includes fluorine atoms. In other embodiments, all of R₁, R₂, and R₃ include fluorine atoms. In certain embodiments, R₃ is fluorine. In other embodiments, R₃ is substituted or unsubstituted, branched or unbranched aliphatic. In yet other embodiments, R₃ is C₁-C₆ alkyl, optionally substituted with a fluorine. In certain embodiments, R₃ is a perfluorinated C₁-C₆ alkyl moiety. In certain particular embodiments, R₃ is methyl. In certain embodiments R₃ is —CF₃, —CHF₂, or —CH₂F.

In certain embodiments, R₃ is of one of the formulae:

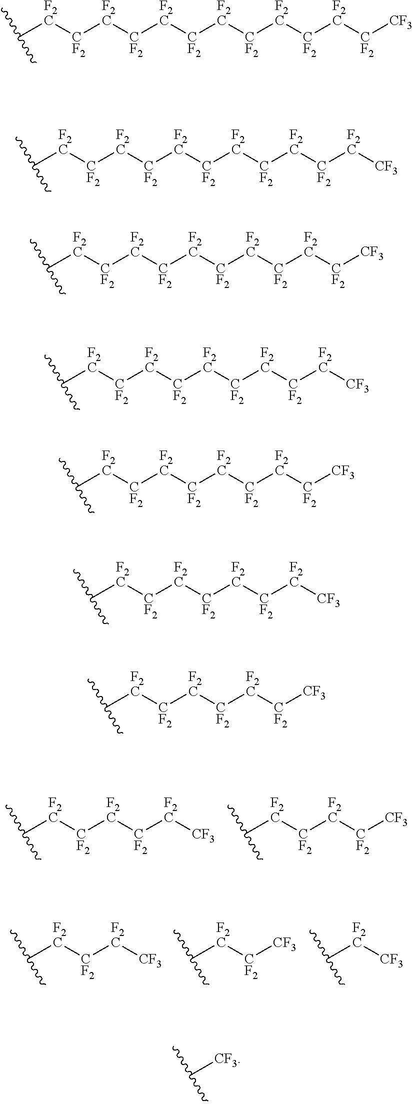

As would be appreciated by one of skill in this art, any of the above perfluorinated alkyl groups may be partially fluorinated, substituted, branched, unsaturated, and/or cyclic. In yet other embodiments, R₃ is a fluorinated, substituted or unsubstituted, branched or unbranched heteroaliphatic moiety. In still other embodiments, R₃ is a fluorinated, substituted or unsubstituted acyl moiety. In other embodiments, R₃ is a fluorinated, substituted or unsubstituted aryl moiety. In certain particular embodiments, R₃ is of the formula:

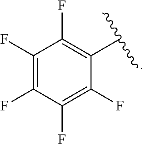

In certain particular embodiments, R₃ is of the formula:

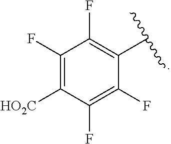

In certain particular embodiments, R₃ is a fluorinated, substituted or unsubstituted phenyl moiety. In certain embodiments, R₃ is fluorinated phenyl (e.g., a phenyl ring with 1, 2, 3, 4, or 5 fluorine substituents). In other embodiments, R₃ is a fluorinated, substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain particular embodiments, R_{C} is —CF₃. In certain embodiments, R_{C} is

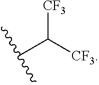

In other embodiments, R_{C} is t-butyl. In certain particular embodiments, R₃ is —CO₂R_{A}, wherein R_{C} is one of the formulae:

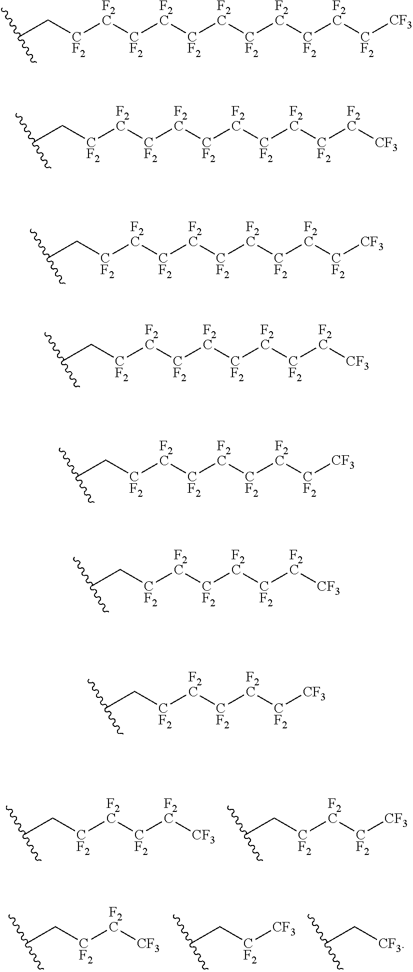

As would be appreciated by one of skill in this art, any of the above fluorinated alkyl groups may be partially fluorinated, substituted, branched, unsaturated, and/or cyclic. In certain particular embodiments, R₃ is —CO₂R_{C}, wherein R_{C} is fluorinated aryl or fluorinated arylalkyl. In certain particular embodiments, R₃ is —CO₂R_{C}, wherein R_{C} is of the formula:

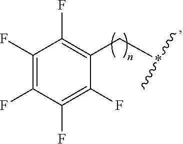

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

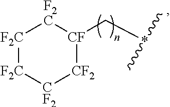

wherein n is an integer between 0 and 12, inclusive. In certain embodiments, n is 0. In certain embodiments, n is 1. In other embodiments, n is 2, 3, 4, 5, or 6.

In certain embodiments, R₁ is —CO₂R_{A}, and R₂ and R₃ are both fluorine. In other embodiments, R₁ is —CO₂R_{A}, and R₂ and R₃ are both methyl. In yet other embodiments, R₁ is —CO₂R_{A}, and R₂ and R₃ are both —CF₃. In certain embodiments, at least one of R₁, R₂, and R₃ is fluorine. In other embodiments, at least two of R₁, R₂, and R₃ are fluorine.

Examplary trisubstituted fluorinated monomers include:

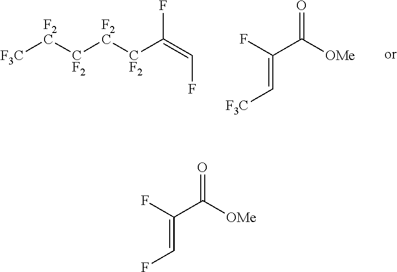

In certain embodiments, the tetrasubstituted fluorinated monomer is of one of the formulae:

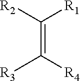

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{A}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —C(═O)N(R_{D})₂; —CN; —SCN; —SR_{D}; —SOR_{D}; —SO₂R_{D}; —NO_{D}; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

wherein R₁, R₂, R₃, or R₄ comprises at least one fluorine atom.

In certain embodiments, R₁ is fluorine. In other embodiments, R₁ is a fluorinated, substituted or unsubstituted, branched or unbranched aliphatic moiety. In certain embodiments, R₁ is of one of the formulae:

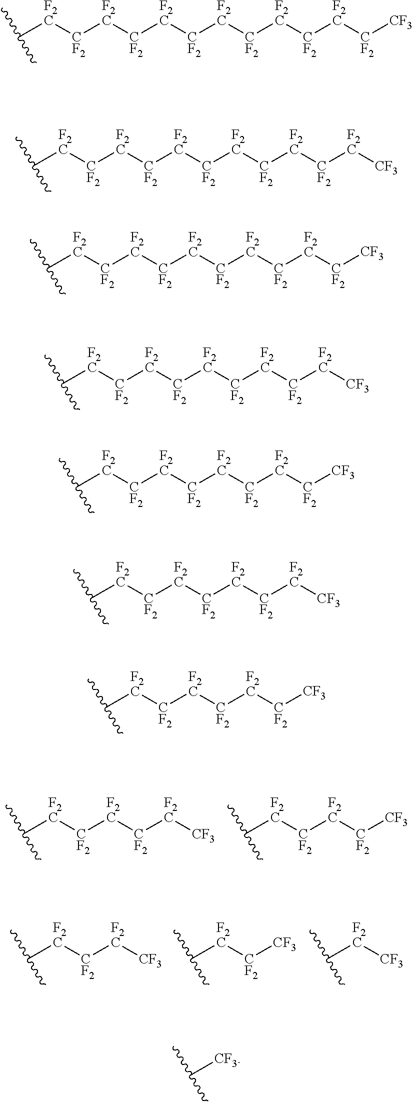

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

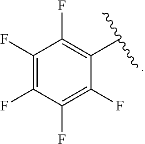

In certain particular embodiments, R₁ is of the formula:

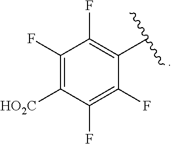

In certain particular embodiments, R₁ is a fluorinated, substituted or unsubstituted phenyl moiety. In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

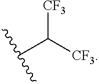

In other embodiments, R_{A} is t-butyl. In certain particular embodiments, R₁ is —CO₂R_{A}, wherein R_{A} is one of the formulae:

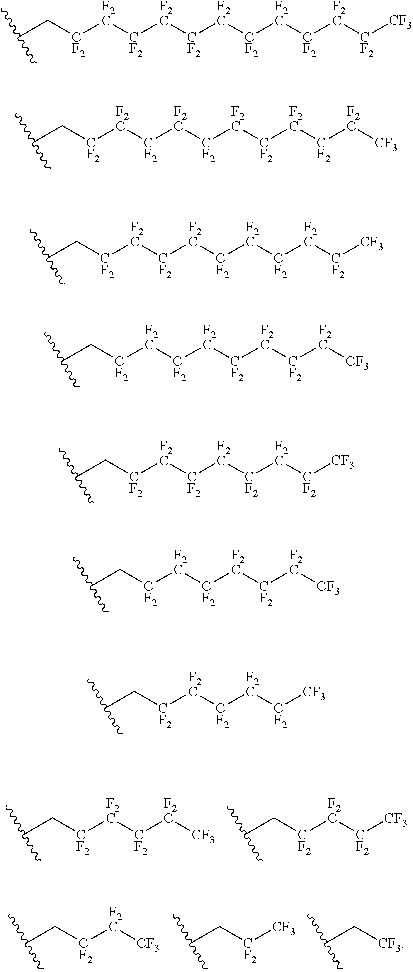

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

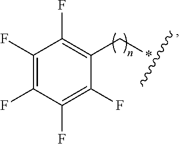

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

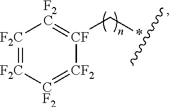

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, R₂ is fluorine. In other embodiments, R₂ is substituted or unsubstituted, branched or unbranched aliphatic. In yet other embodiments, R₂ is C₁-C₆ alkyl, optionally substituted with a fluorine. In certain embodiments, R₂ is a perfluorinated C₁-C₆ alkyl moiety. In certain particular embodiments, R₂ is methyl. In certain embodiments R₂ is —CF₃, —CHF₂, or —CH₂F.

In certain embodiments, R₂ is of one of the formulae:

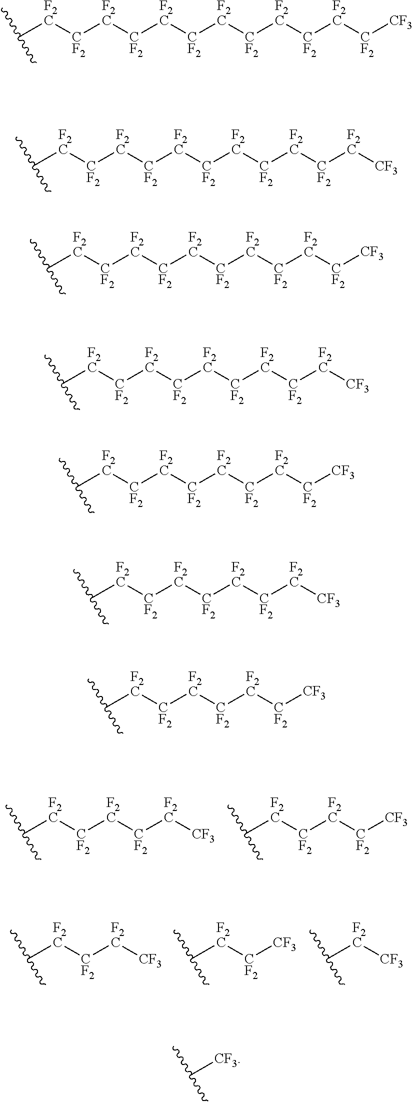

As would be appreciated by one of skill in this art, any of the above perfluorinated alkyl groups may be partially fluorinated, substituted, branched, unsaturated, and/or cyclic. In yet other embodiments, R₂ is a fluorinated, substituted or unsubstituted, branched or unbranched heteroaliphatic moiety. In still other embodiments, R₂ is a fluorinated, substituted or unsubstituted acyl moiety. In other embodiments, R₂ is a fluorinated, substituted or unsubstituted aryl moiety. In certain particular embodiments, R₂ is of the formula:

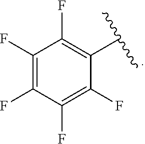

In certain particular embodiments, R₂ is of the formula:

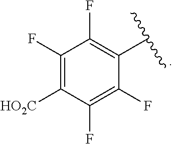

In certain particular embodiments, R₂ is a fluorinated, substituted or unsubstituted phenyl moiety. In certain embodiments, R₂ is fluorinated phenyl (e.g., a phenyl ring with 1, 2, 3, 4, or 5 fluorine substituents). In other embodiments, R₂ is a fluorinated, substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain particular embodiments, R_{B} is —CF₃. In certain embodiments, R_{B} is

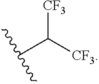

In other embodiments, R_{B} is t-butyl. In certain particular embodiments, R₂ is —CO₂R_{B}, wherein R_{B} is one of the formulae:

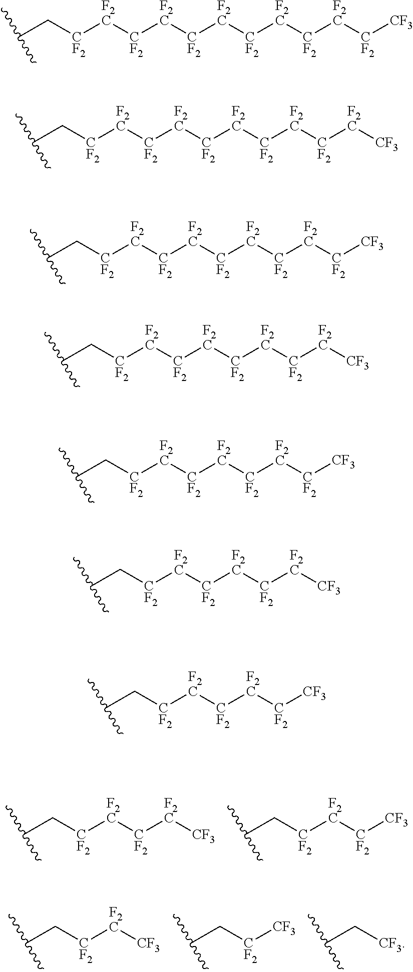

As would be appreciated by one of skill in this art, any of the above fluorinated alkyl groups may be partially fluorinated, substituted, branched, unsaturated, and/or cyclic. In certain particular embodiments, R₂ is —CO₂R_{B}, wherein R_{B} is fluorinated aryl or fluorinated arylalkyl. In certain particular embodiments, R₂ is —CO₂R_{B}, wherein R_{B} is of the formula:

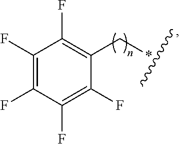

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

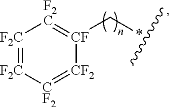

wherein n is an integer between 0 and 12, inclusive. In certain embodiments, n is 0. In certain embodiments, n is 1. In other embodiments, n is 2, 3, 4, 5, or 6.

In certain embodiments, R₃ includes more than 1, 2, 3, 4, 5, 10, 15, 20, or 25 fluorine atoms. In certain embodiments, R₃ is fluorine. In other embodiments, R₃ is substituted or unsubstituted, branched or unbranched aliphatic. In yet other embodiments, R₃ is C₁-C₆ alkyl, optionally substituted with a fluorine. In certain embodiments, R₃ is a perfluorinated C₁-C₆ alkyl moiety. In certain particular embodiments, R₃ is methyl. In certain embodiments R₃ is —CF₃, —CHF₂, or —CH₂F.

In certain embodiments, R₃ is of one of the formulae:

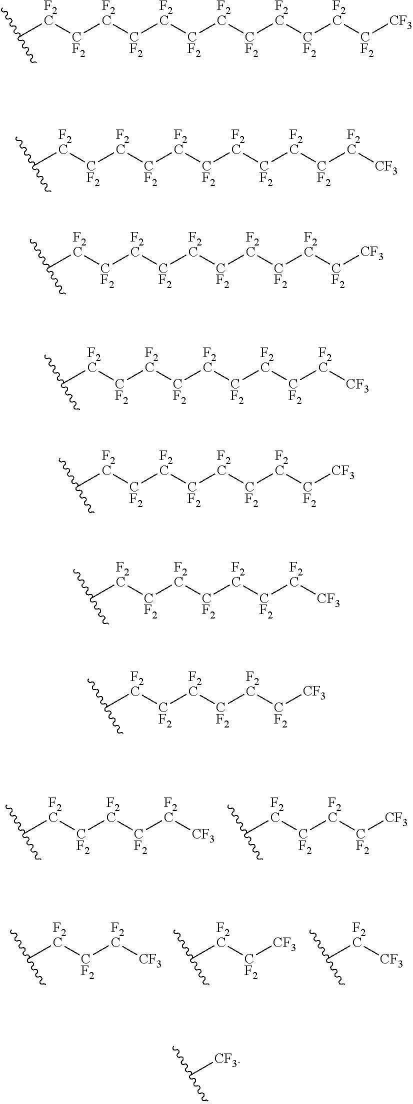

As would be appreciated by one of skill in this art, any of the above perfluorinated alkyl groups may be partially fluorinated, substituted, branched, unsaturated, and/or cyclic. In yet other embodiments, R₃ is a fluorinated, substituted or unsubstituted, branched or unbranched heteroaliphatic moiety. In still other embodiments, R₃ is a fluorinated, substituted or unsubstituted acyl moiety. In other embodiments, R₃ is a fluorinated, substituted or unsubstituted aryl moiety. In certain particular embodiments, R₃ is of the formula:

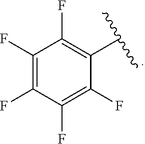

In certain particular embodiments, R₃ is of the formula:

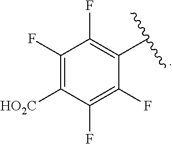

In certain particular embodiments, R₃ is a fluorinated, substituted or unsubstituted phenyl moiety. In certain embodiments, R₃ is fluorinated phenyl (e.g., a phenyl ring with 1, 2, 3, 4, or 5 fluorine substituents). In other embodiments, R₃ is a fluorinated, substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain particular embodiments, R_{C} is —CF₃. In certain embodiments, R_{C} is

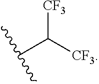

In other embodiments, R_{C} is t-butyl. In certain particular embodiments, R₃ is —CO₂R_{C}, wherein R_{C} is one of the formulae:

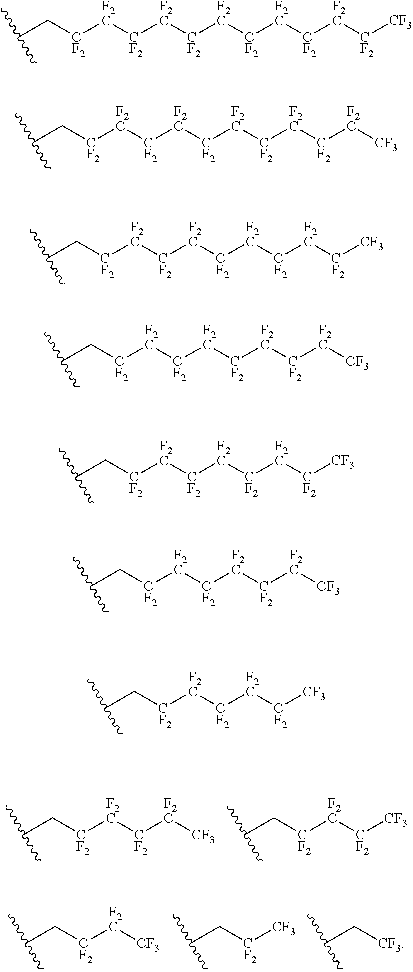

As would be appreciated by one of skill in this art, any of the above fluorinated alkyl groups may be partially fluorinated, substituted, branched, unsaturated, and/or cyclic. In certain particular embodiments, R₃ is —CO₂R_{C}, wherein R_{C} is fluorinated aryl or fluorinated arylalkyl. In certain particular embodiments, R₃ is —CO₂R_{C}, wherein R_{C} is of the formula:

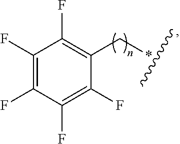

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

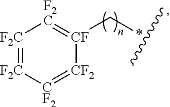

wherein n is an integer between 0 and 12, inclusive. In certain embodiments, n is 0. In certain embodiments, n is 1. In other embodiments, n is 2, 3, 4, 5, or 6.

In certain embodiments, R₄ includes more than 1, 2, 3, 4, 5, 10, 15, 20, or 25 fluorine atoms. In certain embodiments, only one of R₁, R₂, R₃, and R₄ includes fluorine atoms. In certain other embodiments, only two of R₁, R₂, R₃, and R₄ includes fluorine atoms. In certain other embodiments, only three of R₁, R₂, R₃, and R₄ includes fluorine atoms. In other embodiments, all of R₁, R₂, R₃, and R₄ include fluorine atoms. In certain embodiments, R₄ is fluorine. In other embodiments, R₄ is substituted or unsubstituted, branched or unbranched aliphatic. In yet other embodiments, R₄ is C₁-C₆ alkyl, optionally substituted with a fluorine. In certain embodiments, R₄ is a perfluorinated C₁-C₆ alkyl moiety. In certain particular embodiments, R₄ is methyl. In certain embodiments R₄ is —CF₃, —CHF₂, or —CH₂F.

In certain embodiments, R₄ is of one of the formulae:

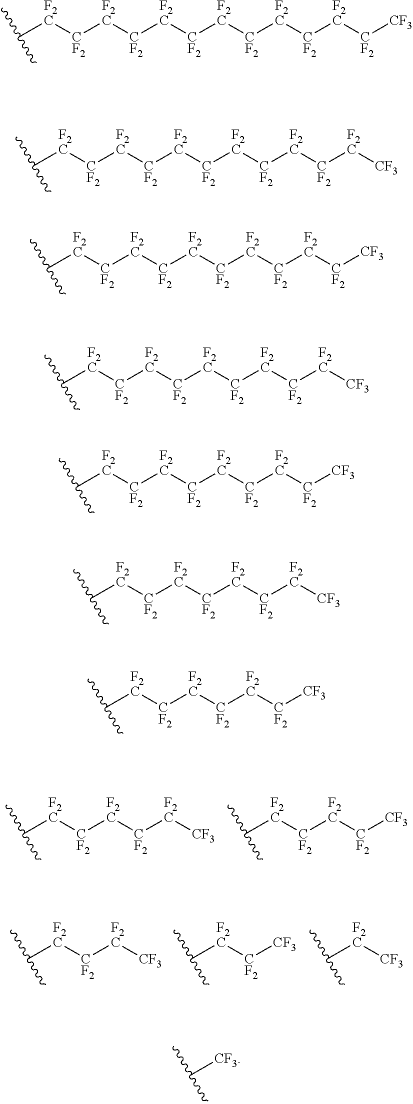

As would be appreciated by one of skill in this art, any of the above perfluorinated alkyl groups may be partially fluorinated, substituted, branched, unsaturated, and/or cyclic. In yet other embodiments, R₄ is a fluorinated, substituted or unsubstituted, branched or unbranched heteroaliphatic moiety. In still other embodiments, R₄ is a fluorinated, substituted or unsubstituted acyl moiety. In other embodiments, R₄ is a fluorinated, substituted or unsubstituted aryl moiety. In certain particular embodiments, R₄ is of the formula:

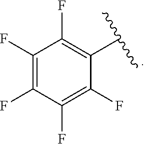

In certain particular embodiments, R₄ is of the formula:

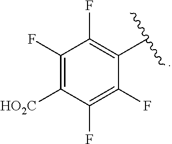

In certain particular embodiments, R₄ is a fluorinated, substituted or unsubstituted phenyl moiety. In certain embodiments, R₄ is fluorinated phenyl (e.g., a phenyl ring with 1, 2, 3, 4, or 5 fluorine substituents). In other embodiments, R₄ is a fluorinated, substituted or unsubstituted heteroaryl moiety. In certain embodiments, R₄ is —C(═O)R_{D}. In other embodiments, R₄ is —CO₂R_{D}. In certain embodiments, R_{D} is C₁-C₆ alkyl. In certain particular embodiments, R_{D} is methyl. In certain particular embodiments, R_{D} is —CF₃. In certain embodiments, R_{D} is

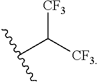

In other embodiments, R_{D} is t-butyl. In certain particular embodiments, R₄ is —CO₂R_{D}, wherein R_{D} is one of the formulae:

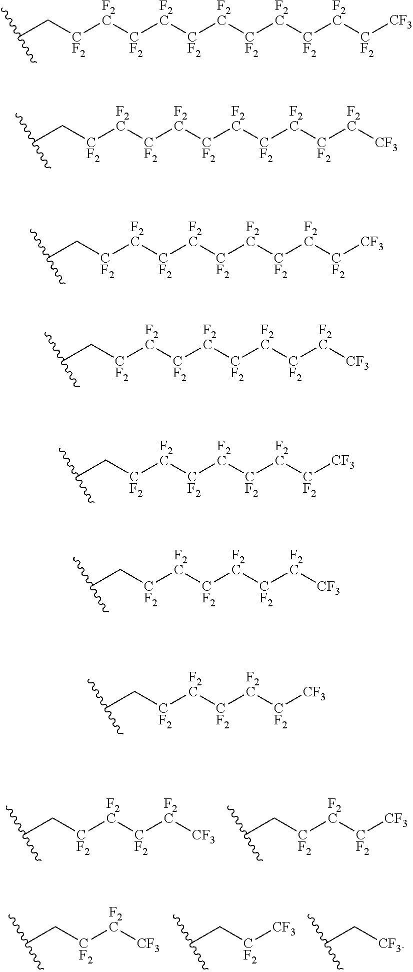

As would be appreciated by one of skill in this art, any of the above fluorinated alkyl groups may be partially fluorinated, substituted, branched, unsaturated, and/or cyclic. In certain particular embodiments, R₄ is —CO₂R_{D}, wherein R_{D} is fluorinated aryl or fluorinated arylalkyl. In certain particular embodiments, R₄ is —CO₂R_{D}, wherein R_{D} is of the formula:

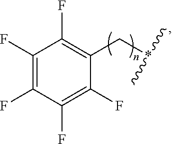

wherein n is an integer between 0 and 12, inclusive. In certain embodiments, n is 0. In certain embodiments, n is 1. In other embodiments, n is 2, 3, 4, 5, or 6. In certain particular embodiments, R₄ is —CO₂R_{D}, wherein R_{D} is of the formula:

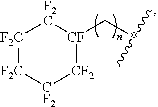

wherein n is an integer between 0 and 12, inclusive. In certain embodiments, n is 0. In certain embodiments, n is 1. In other embodiments, n is 2, 3, 4, 5, or 6.

In certain embodiments, R₁ is —CO₂R_{A}, and R₂ and R₃ are both fluorine. In other embodiments, R₁ is —CO₂R_{A}, and R₂ and R₃ are both methyl. In yet other embodiments, R₁ is —CO₂R_{A}, and R₂ and R₃ are both —CF₃. In certain embodiments, at least one of R₁, R₂, R₃, and R₄ is fluorine. In other embodiments, at least two of R₁, R₂, R₃, and R₄ are fluorine. In other embodiments, at least three of R₁, R₂, R₃, and R₄ are fluorine.

Exemplary tetrasubstituted fluorinated monomers include:

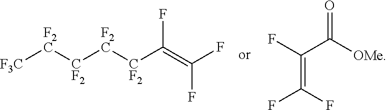

In certain embodiments, the fluorinated monomer is a fluorinated diacrylate or dimethacrylate. In certain embodiments, the fluorinated diacrylate is of the formula:

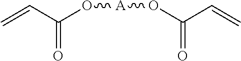

wherein A is a fluorinated linker. In certain embodiments, the fluorinated difluoroacrylate is of the formula:

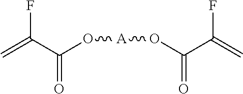

wherein A is a fluorinated linker. In certain embodiments, the fluorinated dimethacrylate is of the formula:

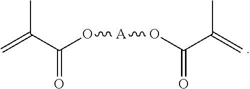

wherein A is a fluorinated linker. In certain embodiments, the fluorinated dimethacrylate is of the formula:

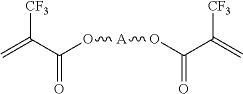

wherein A is a fluorinated linker. In certain embodiments, A is a fluorinated, substituted or unsubstituted, branched or unbranched, cyclic or acyclic aliphatic; fluorinated, substituted or unsubstituted, branched or unbranched, cyclic or acyclic heteroaliphatic; fluorinated, substituted or unsubstituted, aryl; or fluorinated, substituted or unsubstituted, heteroaryl. In certain embodiments, the linker A is a fluorinated alkyl linker. In certain embodiments, the linker A is of one of the formulae:

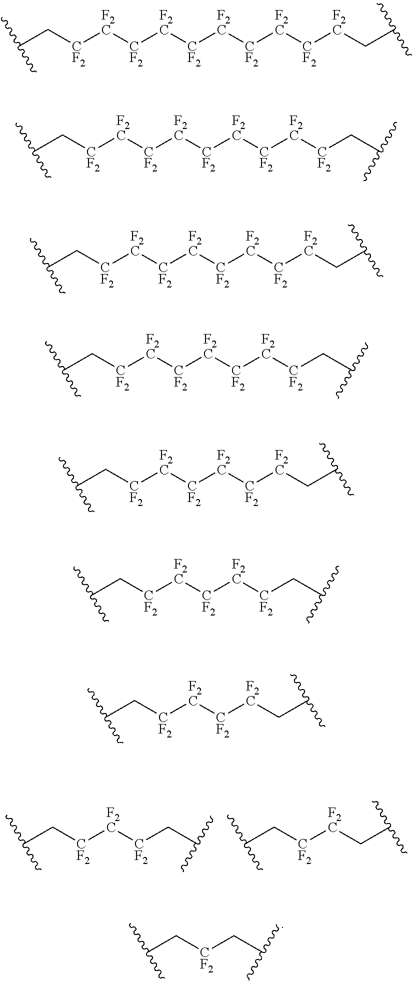

As would be appreciated by one of skill in this art, any of the above fluorinated alkyl groups may be partially fluorinated, substituted, branched, unsaturated, and/or cyclic. In other embodiments, the linker A is of one of the formulae:

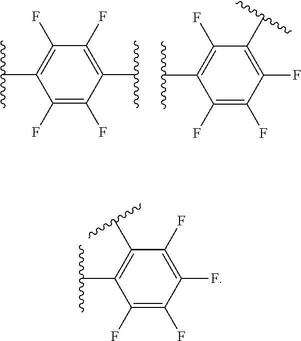

Exemplary diacrylate and dimethacrylates include:

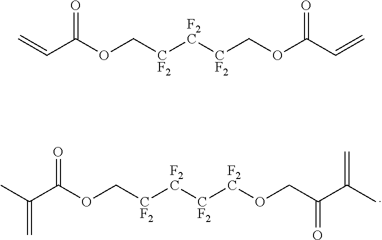

In certain embodiments, the fluorinated monomer is a fluorinated triacrylate or trimethacrylate. In certain embodiments, the fluorinated monomer is of the formula:

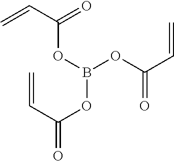

wherein B is fluorinated linker.

In other embodiments, the fluorinated monomer is of the formula:

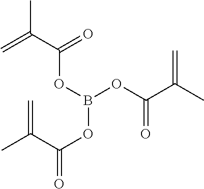

wherein B is fluorinated linker.

In still other embodiments, the fluorinated monomer is of the formula:

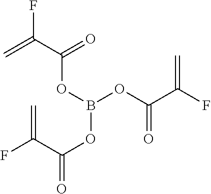

wherein B is fluorinated linker.

In still other embodiments, the fluorinated monomer is of the formula:

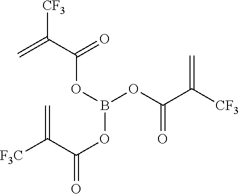

wherein B is fluorinated linker.

In certain embodiments, linker B is a fluorinated, substituted or unsubstituted, branched or unbranched, cyclic or acyclic aliphatic; fluorinated, substituted or unsubstituted, branched or unbranched, cyclic or acyclic heteroaliphatic; fluorinated, substituted or unsubstituted, aryl; or fluorinated, substituted or unsubstituted, heteroaryl. In certain embodiments, the linker B is a branched, fluorinated alkyl linker. In certain embodiments, the linker B is a fluorinated aryl linker. In certain embodiments, the linker B is of the formula:

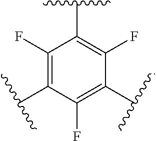

An examplary trimethacrylate is of the formula:

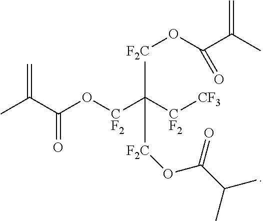

In certain embodiments, the fluorinated monomer is a fluorinated tetraacrylate or tetramethacrylate. Tetraacrylates may be prepared by reacting diacrylates or dimethacrylates with a diamine. An exemplary tetramethacrylate is of the formula:

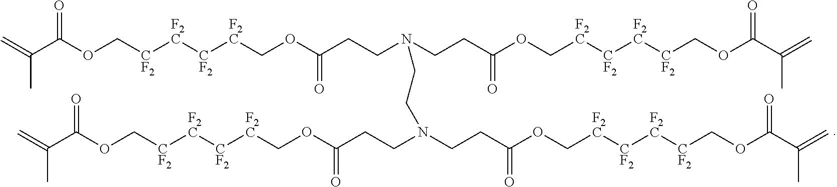

In other embodiments, the fluorinated monomer is a fluorinated pentaacrylate or pentamethacrylate. In still other embodiments, the fluorinated monomer is an even higher acrylate or methacrylate.

In certain embodiments, the fluorinated monomer is a fluorinated alkyne. In certain embodiments, the fluorinated alkynyl monomer is of the formula:

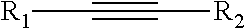

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

wherein R₁ and R₂ comprises at least one fluorine atom.

In certain embodiments, R₁ is fluorine. In certain embodiments, R₁ is hydrogen. In other embodiments, R₁ is a fluorinated, substituted or unsubstituted, branched or unbranched aliphatic moiety. In certain embodiments, R₁ is of one of the formulae:

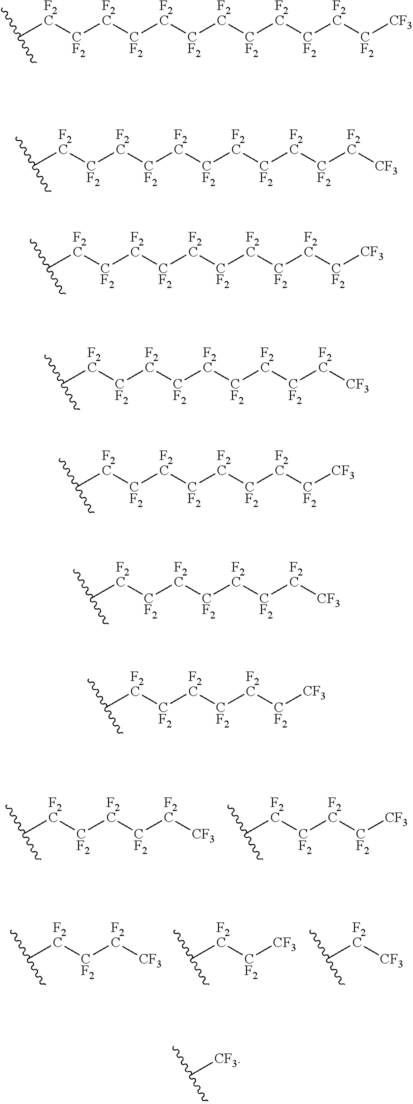

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

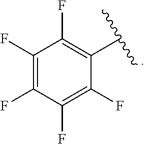

In certain particular embodiments, R₁ is of the formula:

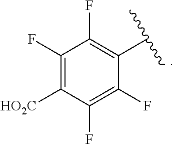

In certain particular embodiments, R₁ is a fluorinated, substituted or unsubstituted phenyl moiety. In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

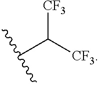

In other embodiments, R_{A} is t-butyl. In certain particular embodiments, R₁ is —CO₂R_{A}, wherein R_{A} is one of the formulae:

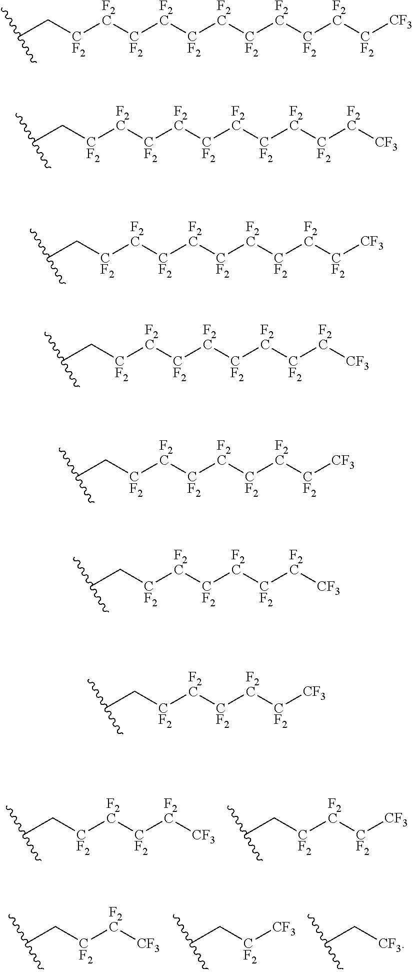

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

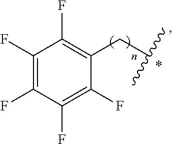

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

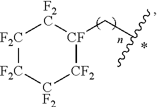

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, only one of R₁ and R₂ includes fluorine atoms. In other embodiments, both R₁ and R₂ include fluorine atoms. In certain embodiments, R₂ is fluorine. In other embodiments, R₂ is hydrogen. In other embodiments, R₂ is substituted or unsubstituted, branched or unbranched aliphatic. In yet other embodiments, R₂ is C₁-C₆ alkyl. In certain embodiments, R₂ is a perfluorinated alkyl moiety. In certain particular embodiments, R₂ is methyl. In certain embodiments R₂ is —CF₃, —CHF₂, or —CH₂F. In certain embodiments, R₂ is a fluorine-substituted aryl or heteroaryl moiety. In certain embodiments, R₂ is a fluorine-substituted phenyl moiety. In certain particular embodiments, R₂ is a perfluorinated phenyl moiety.

In certain embodiments, R₁ is —CO₂R_{A}, and R₂ is fluorine. In certain embodiments, R₁ is —CO₂R_{A}, and R₂ is hydrogen. In other embodiments, R₁ is —CO₂R_{A}, and R₂ is methyl. In yet other embodiments, R₁ is —CO₂R_{A}, and R₂ is —CF₃. In certain embodiments, at least one of R₁ and R₂ is fluorine. In other embodiments, both R₁ and R₂ are fluorine.

Exemplary fluorinated alkynyl monomers include:

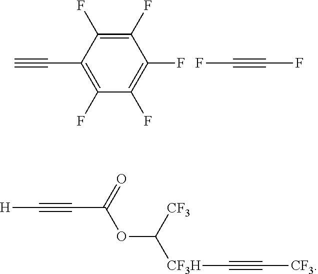

In certain other embodiments, the fluorinated monomer is a fluorinated oligomer. The fluorinated monomers described herein are partially polymerized to form fluorinated oligomers. The fluorinated oligomers are applied to hair and further polymerized on the treated hair. In certain embodiments, the fluorinated oligomers are of a molecular weight sufficient to apply the oligomer to hair. In certain embodiments, the molecular weight of the oligomer is less than 1,000 g/mol. In certain embodiments, the molecular weight is less than 1,500 g/mol. In other embodiments, the molecular weight is less than 2,000 g/mol. In other embodiments, the molecular weight is less than 3,000 g/mol. In other embodiments, the molecular weight is less than 4,000 g/mol. In yet other embodiments, the molecular weight is less than 5,000 g/mol.

In certain embodiments, the fluorinated monomer is mixed with one or more different monomers. The resulting polymer is a co-polymer. As would be appreciated by those of skill in this art, a co-polymer may have desirable properties not attainable with a polymer resulting from the polymerization of one monomer alone. In certain embodiments, two different monomers are applied to hair. In other embodiments, three different monomers are applied to hair. When different monomer are used, the monomers are applied to hair simultaneously or separately. In certain embodiments, the monomers are all in the same solution which is applied to the hair. In certain embodiments, one of the monomers is fluorinated, and another is not fluorinated. In other embodiments, all monomers are fluorinated.

The monomer can be applied to hair using any method. The hair to be treated is brushed, sprayed, rubbed, dipped, soaked, etc. with the monomer or a solution of the monomer. In certain embodiments, the monomer is dissolved in a solvent such as water, alcohol, or other solvent and applied to hair. The solvent may include a propellant such as difluoroethane or dimethyl ether. In certain particular embodiments, the initiator is applied to hair simultaneously with the monomer. In other embodiments, the initiator is applied to hair separately from the monomer. In still other embodiments, the initiator is dissolved in the same solution which contains the monomer. Typically, the concentration of monomer ranges from 0.1% to 10%. In certain embodiments, the initiator is at a concentration ranging from 0.1% to 5%. In certain embodiments, the concentration ranges from 0.1% to 3%. In other embodiments, the concentration of initiator ranges from 0.1% to 2%.

The monomer is typically soluble in a variety of organic solvents (e.g., alcohol), propylene glycol, glycerol, water, or aqueous solutions. In certain embodiments, the initiator is soluble in water or an aqueous solution. An aqueous solution may be acid or basic. In certain embodiments, the initiator is soluble in an alcohol (e.g., methanol, ethanol, denatured ethanol, isopropanol, butanol). Examples of other solvents that can be used for the initiators and/or monomer include, but are not limited to, acetic acid, acetone, alcohol, alcohol (denatured), benzophenone, butoxydiglycol, butyl acetate, n-butyl acetate, n-butyl alcohol, butylene glycol, butyl myristate, butyloctyl benzoate, butyloctyl salicylate, butyl stearate, C12-15 alkyl benzoate, capric acid, caprylic alcohol, cetearyl octanoate, cetyl stearyl octanoate, chlorobutanol, C₉₋₁₁ isoparaffin, C10-11 isoparaffin, C10-13 isoparaffin, decyl alcohol, diethylene glycol, diethylene glycol dibenzoate, diethylhexyl maleate, diethylhexyl 2,6-naphthalate, diethyl sebacate, diisocetyl adipate, diisopopyl adipate, diifiopropyl sebacate, dimethylphthalate, dioctyl adipate, dioctyl succinate, dipropylene glycol, dipropylene glycol dibenzoate, ethoxydiglycol, ethyl acetate, ethyl lactate, ethyl macadamiate, ethyl myristate, ethyl oleate, glycereth-7 benzoate, glycereth-7 diisononanoate, glycereth-4,5-lactate, glycereth-7 triacetate, glycerin, glycine soja (soybean) oil, glycofurol, heptane, hexyl alcohol, hexyldecyl benzoate, hexylene glycol, isobutyl stearate, isocetyl salicylate, isodecyl benzoate, isodecyl isononanoate, isodecyl octanoate, isodecyl oleate, isododecane, isoeicosane, isohexadecane, isononyl isononanoate, isooctane, isopropyl alcohol, isopropyl laurate, isopropyl myristate, isopropyl palmitate, isostearyl stearoyl stearate, laneth-5, lanolin oil, laureth-2 acetate, MEK, methoxydiglycol, methyl acetate, methyl alcohol, methylene chloride, methylpropanediol, methylsoyate, MIBK, morpholine, neopentyl glycol, neopentyl glycol dioctanoate, nonocynol-9, octyl benzoate, octyldodecyl lactate, octyldodecyl octyldodecanoate, octyl isononanoate, octyl isostearate, octyl laurate, octyl palmitate, octyl stearate, oleyl alcohol, olive oil PEG-6 esters, peanut oil PEG-6 esters, PEG-12, PBG-33 castor oil, PEG-50 glyceryl cocoate, PEG-20 hydrogenated castor oil, PEG-6 methyl ether, penetaerythrity tetracaprylate/tetracaprate, pentane, petroleum distillates, polyglyceryl-3 diisostearate, polyglyceryl-2 dioleate, polyoxyethylene glycol dibenzoate, PPG-3, PPG-20 lanolin alcohol ether, PPG-2 myristyl ether propionate, propyl alcohol, propylene carbonate, propylene glycol, propylene glycol caprylate, propylene glycol dibenzoate, propylene glycol methyl ether, propylene glycol myristate, pyridine, ricinus communis (castor) seed oil, sesamum indicum (sesame) oil, sorbitan trioleate, stearyl heptaroate, toluene, 2,2,4-timethylpentane, xylene. In a preferred embodiment, the solvent is selected from the group consisting of propylene glycol, ethanol, isopropanol, n-butanol, water, and mixtures thereof. In certain embodiments, the solvent comprises a mixture of propylene glycol and denatured ethanol. In certain embodiments, the solvent is fluorinated such as 3M Cosmetic Fluid CF-61 or CF-76. As would be appreciated by one of skill in the art, a mixture of more than one solvent in appropriate proportions may be used to deliver the monomer. In certain embodiments, an suspension or emulsion of the monomer is used. In certain embodiments, an emulsifier, detergent, or surfactant is used in the monomer emulsion. In certain embodiments, the surfactant is a fluorinated surfactant (e.g., 3M Novec Fluorosurfactant). In certain embodiments, a propellant is used as at least part of the solvent. Exemplary propellants include difluoroethane and dimethyl ether. In all embodiments, a solvent is optional.

### Polymerization Initiators

The in situ polymerization of the monomers on hair is accomplished via a free radical or ionic polymerization reaction. The polymerization is typically begun using a polymerization initiator. However, in some instances, an initiator may not be used. The polymerization initiator may be chosen based on the type of monomers being used, the type of initiation (e.g., heat or photoinitiation), and solubility of initiator in a solvent or other excipient.

In certain embodiments, the initiator is a free radical initiator, which forms free radicals upon exposure to light or upon heating. Typically, the initiator decomposes upon heating or exposure to a certain wavelength of light to yield two free radicals that initiate the polymerization reaction. The free radical generated from the initiator reacts with an unsaturated functional group (e.g., an alkene, acrylate, or methacrylate functionality) of a monomer thus beginning the chain reaction which results in the formation of the desired fluorinated polymer.

In certain embodiments, the inventive system takes advantage of oxygen tolerant polymerization initiators. Oxygen-tolerant initiators eliminate the need for an oxygen-free or an oxygen-reduced environment for the polymerization reaction to take place. Such oxygen-tolerant initiators allow for the polymerization reaction to take place directly on hair fibers in a normal atmosphere with about 21% oxygen. Exemplary oxygen tolerant polymerization initiators include 4,4′-azobis(4-cyanovaleric acid); 1,1′-azobis(cyclohexanecarbonitrile); 2,2′-azobis(2-methylpropionitrile); benzoyl peroxide; 2,2-bis(tert-butylperoxy)butane; 2,5-bis(tert-butylperoxy)-2,5-dimethylhexane; bis[1-(tert-butylperoxy)-1-methyl ethyl]benzene; tert-butyl hydroperoxide; tert-butyl peracetate; tert-butyl peroxide; tert-butyl peroxybenzoate; cumene hydroperoxide; dicumyl peroxide; lauroyl peroxide; peracetic acid; potassium persulfate; 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone; and 4-methylbenzophenone.

The initiator is applied to hair in the same ways the monomer is applied to hair. The hair to be treated is brushed, sprayed, rubbed, dipped, soaked, etc. with the initiator or a solution of the initiator. In certain embodiments, the initiator is dissolved in a solvent such as water, alcohol, or other cosmetically acceptable solvent, and applied to hair. In certain particular embodiments, the initiator is applied to hair simultaneously with the monomer. In other embodiments, the initiator is applied to hair separately from the monomer. In this case, the solvent for the monomer may be different that the solvent used for the polymerization initiator. The monomer and initiator may be applied in any order. In still other embodiments, the initiator is dissolved in the same solution which contains the monomer. The initiator is typically at a lower concentration in the solution than the monomer. Typically, the concentration of initiator is approximately 1000-fold, 100-fold, 10-fold, or 5-fold less than the concentration of monomer. In certain embodiments, the initiator is at a concentration ranging from 0.001% to 10%. In certain embodiments, the initiator is at a concentration ranging from 0.001% to 5%. In certain embodiments, the concentration ranges from 0.01% to 1%. In other embodiments, the concentration of initiator ranges from 0.1% to 1%. In certain embodiments, when a high concentration of polymerization initiator is needed, the initiator may be applied neat (i.e., without a solvent).

The initiator is typically soluble in a variety of organic solvents (e.g., alcohol, denatured ethanol, isopropanol), propylene glycol, glycerol, water, or aqueous solutions. Selection of an acceptable solvent will depend on the initiator as well as the method of application. Typically an acceptable solvent will not adversely impact the in situ polymerization process.

In certain embodiments, the initiator is soluble in water or an aqueous solution. An aqueous solution may be acid or basic. In certain embodiments, the initiator is soluble in an alcohol (e.g., methanol, ethanol, denatured ethanol, isopropanol, butanol). Examples of other solvents that can be used for the initiators and/or monomer include, but are not limited to, acetic acid, acetone, alcohol, alcohol (denatured), benzophenone, butoxydiglycol, butyl acetate, n-butyl acetate, n-butyl alcohol, butylene glycol, butyl myristate, butyloctyl benzoate, butyloctyl salicylate, butyl stearate, C12-15 alkyl benzoate, capric acid, caprylic alcohol, cetearyl octanoate, cetyl stearyl octanoate, chlorobutanol, C₉₋₁₁ isoparaffin, C10-11 isoparaffin, C10-13 isoparaffin, decyl alcohol, diethylene glycol, diethylene glycol dibenzoate, diethylhexyl maleate, diethylhexyl 2,6-naphthalate, diethyl sebacate, diisocetyl adipate, diisopopyl adipate, diisopropyl sebacate, dimethylphthalate, dioctyl adipate, dioctyl succinate, dipropylene glycol, dipropylene glycol dibenzoate, ethoxydiglycol, ethyl acetate, ethyl lactate, ethyl macadamiate, ethyl myristate, ethyl oleate, glycereth-7 benzoate, glycereth-7 diisononanoate, glycereth-4,5-lactate, glycereth-7 triacetate, glycerin, glycine soja (soybean) oil, glycofurol, heptane, hexyl alcohol, hexyldecyl benzoate, hexylene glycol, isobutyl stearate, isocetyl salicylate, isodecyl benzoate, isodecyl isononanoate, isodecyl octanoate, isodecyl oleate, isododecane, isoeicosane, isohexadecane, isononyl isononanoate, isooctane, isopropyl alcohol, isopropyl laurate, isopropyl myristate, isopropyl palmitate, isostearyl stearoyl stearate, laneth-5, lanolin oil, laureth-2 acetate, MEK, methoxydiglycol, methyl acetate, methyl alcohol, methylene chloride, methylpropanediol, methylsoyate, MIBK, morpholine, neopentyl glycol, neopentyl glycol dioctanoate, nonocynol-9, octyl benzoate, octyldodecyl lactate, octyldodecyl octyldodecanoate, octyl isononanoate, octyl isostearate, octyl laurate, octyl palmitate, octyl stearate, oleyl alcohol, olive oil PEG-6 esters, peanut oil PEG-6 esters, PEG-12, PBG-33 castor oil, PEG-50 glyceryl cocoate, PEG-20 hydrogenated castor oil, PEG-6 methyl ether, penetaerythrity tetracaprylate/tetracaprate, pentane, petroleum distillates, polyglyceryl-3 diisostearate, polyglyceryl-2 dioleate, polyoxyethylene glycol dibenzoate, PPG-3, PPG-20 lanolin alcohol ether, PPG-2 myristyl ether propionate, propyl alcohol, propylene carbonate, propylene glycol, propylene glycol caprylate, propylene glycol dibenzoate, propylene glycol methyl ether, propylene glycol myristate, pyridine, ricinus communis (castor) seed oil, sesamum indicum (sesame) oil, sorbitan trioleate, stearyl heptaroate, toluene, 2,2,4-timethylpentane, and xylene. In a preferred embodiment, the solvent is selected from the group consisting of propylene glycol, ethanol, isopropanol, n-butanol, water, and mixtures thereof. As would be appreciated by one of skill in the art, a mixture of more than one solvent in appropriate proportions may be used to deliver the initiator(s) and/or monomer(s). In certain embodiments, a propellant such as difluoroethane or dimethyl ether is used as at least part of the solvent. In certain embodiments, the solvent is fluorinated such as 3M Cosmetic Fluid CF-61 or CF-76. In all embodiments, a solvent is optional.

The initiator for the polymerization reaction is typically chosen based on a variety of concerns including the structure of the monomer, toxicity, biocompatibility, solubility, heat versus photoinitiation, tolerance to oxygen, tolerance to water, etc. In certain embodiments, the initiator is compatible with initiating polymerization of at least one of the polymerizable monomers to be used in the hair treatment. In certain particular embodiments, the initiator is oxygen tolerant. In certain embodiments, the initiator is non-toxic. In other embodiments, the initiator is biocompatible. In certain embodiments, the initiator is oxygen tolerant. These and other concerns may be taken into account by one of skill in the art choosing the initiator to be used. The initiator may be obtained from a commercial source such as Sigma-Aldrich, Ciba-Geigy, Sartomer, etc. The initiator may also be prepared synthetically.

The inventive system may include the use of one or more polymerization initiators. In certain embodiments, 2, 3, 4, or more polymerization initiators are used. In certain embodiments, one polymerization initiator is used. In certain embodiments, two polymerization initiators are used. In certain embodiments, three polymerization initiators are used. In certain embodiments, more than one initiator is used, and each of the initiators is used to initiate the polymerization of a different monomer being used in the treatment. The difference polymerization initiators may be provided for application to hair in different or the same composition with or without monomer.

In certain embodiments, the initiator is a free radical thermal initiator. Any thermal initiator may be used in the polymerization reaction. In certain embodiments, the thermal initiator is designed to work at a temperature ranging from 30° C. to 120° C. In certain embodiments, the initiator is designed to work at a temperature ranging from 30° C. to 100° C. In other embodiments, the initiator is designed to work at a temperature ranging from 30° C. to 80° C. In certain embodiments, the initiator is designed to work at a temperature ranging from 40° C. to 70° C. In certain particular embodiments, the initiator is designed to work at approximately 30, 40, 50, 60, 70, 80, 90, 100, or 110° C. In certain embodiments, a co-initiator is used. Co-initiators act to lower the decomposition temperature of the initiator. Exemplary co-initiators include, but are not limited to, aromatic amine (e.g., dimethyl aniline), organic peroxides, decahydroacridine 1,8-dione, etc. Other co-initiators are list below. The heat may be applied to hair with monomer and initiator applied for about 10 seconds to about 5 minutes. In certain embodiments, the heat is applied for about 10 to about 60 seconds. In other embodiments, the heat is applied for about 10 to about 30 seconds. In yet other embodiments, the heat is applied for about 20 to about 40 seconds. The heat source for initiating polymerization may include, but is not limited, to blow dryers, curling irons, hot curlers, hair irons, hair straighteners, hair crimpers, hot air brushes, and hair dryers.

Thermal initiators include peroxides, peracids, peracetates, persulfates, etc. Exemplary thermal initiators include tert-amyl peroxybenzoate; 4,4′-azobis(4-cyanovaleric acid); 1,1′-azobis(cyclohexanecarbonitrile); 2,2′-azobis(2-methylpropionitrile); benzoyl peroxide; 2,2′-azo-bis-isobutyronitrile (AIBN); benzoyl peroxide; 2,2-bis(tert-butylperoxy)butane; 1,1-bis(tert-butylperoxy)cyclohexane; 2,5-bis(tert-butylperoxy)-2,5-dimethylhexane; 2,5-bis(tert-butylperoxy)-2,5-dimethyl-3-hexyne; bis[1-(tert-butylperoxy)-1-methylethyl]benzene; 1,1-bis (tert-butylperoxy)-3,3,5-trimethylcyclohexane; tert-butyl hydroperoxide; tert-butyl peracetate; tert-butyl peracetic acid; tert-butyl peroxide; tert-butyl peroxybenzoate; tert-butylperoxy isopropyl carbonate; cumene hydroperoxide; cyclohexanone peroxide; dicumyl peroxide; lauroyl peroxide; 2,4-pentanedione peroxide; peracetic acid; and potassium persulfate. Many of the above listed thermal initiators are available from commercial sources such as Sigma-Aldrich. In certain embodiments, the initiator is 2,2′-azo-bis-isobutyronitrile (AIBN). In other embodiments, the initiator is benzoyl peroxide (also known as dibenzoyl peroxide). In certain embodiments, a combination of thermal initiators is used. In certain embodiments, the polymerization initiator is a combination of ammonium persulfate (APS) and N,N,N′,N′-tetramethylethylenediamine (TEMED).

In other embodiments, the free radical initiator is a photoinitiator. Photoinitiators produce reactive free radical species that initiate the polymerization of monomers upon exposure to light. Any photoinitiator may be used in the polymerization reaction. Photoinitiated polymerizations and photoinitiators are discussed in detail in Rabek, Mechanisms of Photophysical Processes and Photochemical Reactions in Polymers, New York: Wiley & Sons, 1987; Fouassier, Photoinitiation, Photopolymerization, and Photocuring, Cincinnati, Ohio: Hanser/Gardner; Fisher et al., “Photoinitiated Polymerization of Biomaterials” Annu. Rev. Mater. Res. 31:171-81, 2001; incorporated herein by reference. The photoinitiator may be designed to produce free radicals at any wavelength of light. In certain embodiments, the photoinitiator is designed to work using UV light (200-500 nm). In certain particular embodiments, the photoinitator is designed to work using UV light with a wavelength of approximately 365 nm. In certain embodiments, long UV rays are used. In other embodiments, short UV rays are used. In other embodiments, the photoinitiator is designed to work using visible light (400-800 nm). In certain embodiments, the photoinitiator is designed to work using blue light (420-500 nm). In yet other embodiments, the photoinitiator is designed to work using IR light (800-2500 nm). The output of light can be controlled to provide greater control over the polymerization reaction. Control over the polymerization reaction in turn results in control over the hair treatment or hair style. In certain embodiments, the intensity of light ranges from about 500 to about 10,000 μW/cm². In certain embodiments, the intensity of light is about 4000, 5000, 6000, 7000, 8000, or 9000 μW/cm². The light may be applied to hair with monomer and initiator applied for about 10 seconds to about 5 minutes. In certain embodiments, the light is applied for about 10 to about 60 seconds. In other embodiments, the light is applied for about 10 to about 30 seconds. In yet other embodiments, the light is applied for about 20 to about 40 seconds. The light source may allow variation of the wavelength of light and/or the intensity of the light. Light sources useful in the inventive system include, but are not limited to, lamps, fiber optics devices, brushes with light sources, and styling devices with light sources.

In certain embodiments, the photoinitiator is a peroxide (e.g., ROOR′). In other embodiments, the photoinitiator is a ketone (e.g., RCOR′). In other embodiments, the compound is an azo compound (e.g., compounds with a —N═N— group). In certain embodiments, the photoinitiator is an acylphosphineoxide. In other embodiments, the photoinitiator is a sulfur-containing compound. In still other embodiments, the initiator is a quinone. Exemplary photoinitiators include acetophenone; anisoin; anthraquinone; anthraquinone-2-sulfonic acid, sodium salt monohydrate; (benzene)tricarbonylchromium; 4-(boc-aminomethyl)phenyl isothiocyanate; benzin; benzoin; benzoin ethyl ether; benzoin isobutyl ether; benzoin methyl ether; benzoic acid; benzophenone; benzyl dimethyl ketal; benzophenone/1-hydroxycyclohexyl phenyl ketone; 3,3′,4,4′-benzophenonetetracarboxylic dianhydride; 4-benzoylbiphenyl; 2-benzyl-2-(dimethylamino)-4′-morpholinobutyrophenone; 4,4′-bis(diethylamino)benzophenone; 4,4′-bis(dimethylamino)benzophenone; Michler's ketone; camphorquinone; 2-chlorothioxanthen-9-one; 5-dibenzosuberenone; (cumene)cyclopentadienyliron(II) hexafluorophosphate; dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; 11-mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenyldithiocarbonate; 2,2′-azobisisobutyronitrile (AIBN); camphorquinone (CQ); eosin; dimethylaminobenzoate (DMAB); dimethoxy-2-phenyl-acetophenone (DMPA); Quanta-cure ITX photosensitizer (Biddle Sawyer); Irgacure 907 (Ciba Geigy); Irgacure 651 (Ciba Geigy); Darocur 2959 (Ciba Geigy); ethyl-4-N,N-dimethylaminobenzoate (4EDMAB); 1-[4-(4-benzoylphenylsulfanyl)phenyl]-2-methyl-2-(4-methylphenylsulfonyl)propan-1-one; 1-hydroxy-cyclohexyl-phenyl-ketone; 2,4,6-trimethylbenzoyldiphenylphosphine oxide; diphenyl(2,4,6-trimethylbenzoyl)phosphine; 2-ethylhexyl-4-dimethylaminobenzoate; 2-hydroxy-2-methyl-1-phenyl-1-propanone; 65% (oligo[2-hydroxy-2-methyl-1-[4-(1-methylvinyl)phenyl]propanone] and 35% propoxylated glyceryl triacrylate; benzil dimethyl ketal; benzophenone; blend of benzophenone and a-hydroxy-cyclohexyl-phenyl-ketone; blend of Esacure KIP150 and Esacure TZT; blend of Esacure KIP150 and Esacure TZT; blend of Esacure KIP150 and TPGDA; blend of phosphine oxide, Esacure KIP150 and Esacure TZT; difunctional a-hydroxy ketone; ethyl 4-(dimethylamino)benzoate; isopropyl thioxanthone; 2-hydroxy-2-methyl-phenylpropanone; 2,4,6,-trimethylbenzoyldiphenyl phosphine oxide; 2,4,6-trimethyl benzophenone; liquid blend of 4-methylbenzophenone and benzophenone; oligo(2-hydroxy-2 methyl-1-(4(1-methylvinyl)phenyl)propanone; oligo(2-hydroxy-2-methyl-1-4 (1-methylvinyl)phenyl propanone and 2-hydroxy-2-methyl-1-phenyl-1-propanone (monomeric); oligo (2-hydroxy-2-methyl-1-4 (1-methylvinyl)phenyl propanone and 2-hydroxy-2-methyl-1-phenyl-1-propanone (polymeric); 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone. In certain embodiments, the photoinitiator is acetophenone; diphenyl(2,4,6-trimethylbenzoyl)phosphine oxide; 4,4′-dimethoxybenzoin; anthraquinone; anthraquinone-2-sulfonic acid; benzene-chromium(0) tricarbonyl; 4-(boc-aminomethyl)phenyl isothiocyanate; benzil; benzoin; benzoin ethyl ether; benzoin isobutyl ether; benzoin methyl ether; benzophenone; benzoic acid; benzophenone/1-hydroxycyclohexyl phenyl ketone, 50/50 blend; benzophenone-3,3′,4,4′-tetracarboxylic dianhydride; 4-benzoylbiphenyl; 2-benzyl-2-(dimethylamino)-4′-morpholinobutyrophenone; 4,4′-bis(diethylamino)benzophenone; Michler's ketone; (+)-camphorquinone; 2-chlorothioxanthen-9-one; 5-dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 3,4-dimethylbenzophenone; diphenyl(2,4,6-trimethylbenzoyl)phosphine oxide/2-hydroxy methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methyl benzoylformate; 2-methyl-4′-(methylthio)-2-morpholinopropiophenone; 9,10-phenanthrenequinone; 4′-phenoxyacetophenone; thioxanthen-9-one; triarylsulfonium hexafluorophosphate salts; 3-mercapto-1-propanol; 11-mercapto-1-undecanol; 1-mercapto-2-propanol; and 3-mercapto-2-butanol, all of which are commercially available from Sigma-Aldrich. In certain embodiments, the free radical initiator is selected from the group consisting of benzophenone, benzyl dimethyl ketal, 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone and 4-methylbenzophenone. In certain embodiments, the photoinitiator is dimethoxy-2-phenyl-acetophenone (DMPA). In certain embodiments, the photoinitiator is a titanocene. In certain embodiments, a combination of photoinitiators is used.

In other embodiments, an initiator of a cationic or anionic polymerization process is used. In certain embodiments, the initiator is a photoinitiator of a cationic polymerization process. Exemplary photoinitiators of cationic polymerization include, but are not limited to, titanium tetrachloride, vanadium tetrachloride, bis(cyclopentadienyl)titanium dichloride, ferrocene, cyclopentadienyl manganese tricarbonyl, manganese decacarbonyl, diazonium salts, diaryliodonium salts (e.g., 3,3′-dinitrodiphenyliodonium hexafluoroarsenate, diphenyliodonium fluoroborate, 4-methoxydiphenyliodonium fluoroborate) and triarylsulfonium salts. In certain embodiments, a hybrid free radical/cationic photopolymerization is used to polymerize the monomers in situ on hair.

### Polymerization Reaction and Use

The monomer(s) and initiator(s) as discussed above are applied to hair to be treated using the inventive system. The monomers are then polymerized on the hair using light or heat to initiate the polymerization reaction. The amount of light and heat, as described above, will depend on the monomers and initiator being used, the styling of the hair, concentration of the initiator, concentration of the monomer, etc. Basic guidelines are provided herein for the inventive system using various initiator; however, these guidelines may be adjusted by one of skill in the art to provide the desired results.

According the methods of the invention, the hair to be treated is optionally washed to remove any excess dirt or oil before the treatment is begun. The monomer and polymerization initiator is then applied to the hair by any technique known in the art including spraying, dipping, washing, brushing, rubbing, etc. As described above, the monomer and polymerization initiator may be applied together or separately. The compositions for application to hair may include some or all of the following properties: good consistency, good distributability, economical application, good definition and texture, slight load, good strength, lack of undesired residue, ease of shaping hair, and suitable drying time. After both have been applied to the hair, the hair is exposed to light or heat to initiate the in situ polymerization process. In certain embodiments, the monomers are polymerized concomitantly with the application of the monomer and initiator. In certain embodiments, the monomers are polymerized both concomitantly with application of the monomer and initiator and subsequent to the application. In other embodiments, the hair is allowed to dry before the polymerization reaction is begun. In other embodiments, the polymerization is started as soon as the monomer is applied to the hair. In certain embodiments, the application and polymerization steps are repeated until the desired hair characteristic is achieved. In certain embodiments, the polymerization process results in a branched or cross-linked polymer which results in a stronger polymer. The inventive system may be used to style and/or produce a desired cosmetic effect. In certain embodiments, the desired characteristic is luster, shine, smoothness, slip, static control, feel, straightening, curl, waviness, etc. In certain embodiments, the inventive hair care system is used to straighten wavy, frizzy, or curly hair. In other embodiments, the inventive system is used to restore luster and/or smoothness to hair. In other embodiments, the inventive system is used to control static in hair. In yet other embodiments, the inventive system is used to give hair a distinct feel. In certain embodiments, the treatment is used to color hair. In other embodiments, the treatment is used to restore damaged hair. In other embodiments, the treatment is used to style hair. In yet other embodiments, the treatment is used to give hair body. In other embodiments, the treatment is used to curl hair or give hair a wave. In certain embodiments, the treatment is used to straighten hair.

Moreover, instead of or in addition to simply imparting and maintaining a physical hair style, the composition applied to hair may include dyes, thereby resulting in a color treatment. The dyes may be covalently associated with the components of the compsition such as the monomers. In such case, the dye may become part of the polymer. In other embodiments, the dye is separate but may become entrapped in the polymeric matrix formed on the hair fiber. Furthermore, other compounds conducive to hair treatment may be used in the inventive system. For example, vitamins, and lipids may be included in the composition applied to hair. In certain embodiments, the inventive system is used to deliver agents that strengthen hair. In certain embodiments, the inventive system is used to deliver agents that enhance hair elasticity. In certain embodiments, the inventive system is used to deliver agents known in the art to enhance the optical properties of hair (e.g., shine, color). The inventive system may facilitate transcuticular delivery of agents.

The inventive system may be used on any animal with hair. The system is particularly useful for treating human hair. However, the hair of other mammals may also be treated. For example, the hair of domesticated animals such as dogs and cats may be treated using the inventive system. In addition, the hair of test animals such as rodents (e.g., mouse, rat, rabbit, guinea pig, etc.) or primates may also be treated. In certain embodiments, hair samples from a human (e.g., hair clippings) or other animals are tested with the inventive system using different monomers, initiators, etc. Hair samples treated with the inventive system are considered to be within the scope of the invention. These hair samples comprise polymers on the hair. In certain embodiments, the hair is human hair. In other embodiments, the hair is non-human hair. In certain embodiments, the hair is dog or cat hair. In other embodiments, the hair is rat, mouse, guinea pig, rabbit, gerbil, or primate hair. The hair treatment system of the present invention can also be used to treat hair contained in wigs, toupees, and hairpieces. In certain embodiments, the polymers have been formed (i.e., polymerized) in situ using the inventive method of polymerizing fluorinated monomers on hair.

The in situ polymerization process can be initiated by a light or heat source. In certain embodiments, a light source is used. The light source may be an IR, visible, or UV light source. The wavelength(s) of light generated by the light source should typically correspond with the wavelength of light for activating the polymerization initiator used. The light source may allow for generation of light of varying wavelengths and intensity. Varying the output of light allows for greater control of the polymerization process.

In certain embodiments, the light source is an IR light source. In other embodiments, the light source is a visible light source. In still other embodiments, the light source is a UV light source. In certain embodiments, the light source emits light with a wavelength of about 200 nm to about 600 nm and an intensity of about 500 μW/cm² to about 10,000 μW/cm². In certain particular embodiments, the light source emits light at a wavelength of 365 nm and at an intensity of about 7,000 μW/cm². In certain embodiments, the light source emits light at an intensity of about 4000, 5000, 6000, 7000, 8000, or 9000 μW/cm². In certain embodiments, the light source emits light at a wavelength of about 200 to about 400 nm. The light may be applied to the hair concurrently with the application of monomer and/or polymerization initiator and/or subsequent to application of monomer and/or polymerization initiator. The treated hair is exposed to the light source from 5 seconds to 60 seconds. In certain embodiments, the exposure is about 10 seconds to about 30 seconds. In certain embodiments, the exposure is about 20 seconds to about 40 seconds. In certain embodiments, the exposure is about 30 seconds. In certain embodiments, the exposure is about 60 seconds.

In certain embodiments, a heat source is used to initiate the in situ polymerization process. Examples of heat sources that may be used include blow dryers, curling irons, flattening irons, hot curlers, hair dryers, and heat lamps. The output temperature of the heat source is typically in the range of about 50° C. to about 500° C. In certain embodiments, the output temperature of the heat source is from about 50° C. to about 200° C. In certain embodiments, the output temperature of the heat source is from about 50° C. to about 100° C. The heat source may heat the hair to a temperature of about 30° C. to about 120° C. In certain embodiments, the temperature is about 40° C. to about 70° C. In certain embodiments, the temperature is about 45° C. to about 80° C. In certain embodiments, the temperature is about 40° C. to about 50° C. In certain embodiments, the temperature is about 50° C. to about 60° C. In certain embodiments, the temperature is about 50° C. to about 70° C. In certain embodiments, the temperature is about 60° C. to about 80° C. In certain embodiments, the temperature is about 70° C. to about 90° C. In certain embodiments, the temperature is about 90° C. to about 120° C. The treated hair is exposed to the heat source from 5 seconds to 120 seconds. In certain embodiments, the exposure is about 10 seconds to about 60 seconds. In certain embodiments, the exposure is about 20 seconds to about 60 seconds. In certain embodiments, the exposure is about 30 seconds. In certain embodiments, the exposure is about 60 seconds. In certain embodiments, the exposure is about 90 seconds. In certain embodiments, the exposure is about 120 seconds.

Without wishing to be bound by any particular theory, the polymerization reaction is thought to cause the polymerization of the monomers on the hair of the subject being treated. The polymerization reaction may also lead to the covalent attachment of polymer to the hair (e.g., keratin, other proteins, lipids, or carbohydrates found in hair). The formed polymer may fill in gaps, cracks, ridges, holes, splits, pits, etc. in the hair. The inventive system is particularly useful for treating hair with polymers that could not otherwise be applied to hair using conventional means because of solubility issues.

### Kits

The invention also provides kits for use in treating hair based on the inventive system for the in situ polymerization of fluorinated monomers on hair. The kit may include all or a portion of the components necessary to treat hair. In certain embodiments, the kit includes enough product to teat one head of hair. In other embodiments, the kit include enough product to treat multiple heads of hair (e.g., approximately 2, 3, 4, 5, 10, 15, 20, 25, or 50 heads of hair). The kit may include any or all of the following components: monomers, photoinitiators, thermal initiators, solvent (e.g., ethanol, denatured ethanol, propylene glycol), water, vials, heat source, light source, spray bottle, brush, hair dryer, curling iron, containers, and instructions for use. The compositions of the kit may be packaged as lotions, mousses, solutions, gels, emulsions, suspensions, pumpable hair sprays, aerosol sprays, and non-aerosol sprays (e.g., atomisers). Compositions of the kit such as monomer and/or initiator compositions are typically conveniently packaged in a suitable container for shipping and/or application of the composition. For example, a monomer composition may be provided in a pump spray bottle or spray can. In certain embodiments, the components of the kits are conveniently packaged for use by the end use along with instructions for use in accordance with the present invention. The kit may or may not include a heat source or light source. In certain embodiments, the kit is tailored for producing a desired characteristic in the treated hair. The kit may also include other hair care products including dyes, shampoo, conditioner, gel, mousse, etc.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### In Situ Polymerization of Acrylate and Vinyl Monomers on Hair

In this example, a solution (designated F2) was prepared containing the pentaacrylate ester SR9041 (Sartomer) (1% w/w), a free radical photoinitiator (1% w/w) KT046 (Sartomer) (1% w/w), propylene glycol (2.25% w/w), and denatured ethanol (95.6% w/w). This solution was applied to human hair and curled with a hot curling iron under UV irradiation using a Black Lamp UV source (λ=365 nm, intensity=7000 μW/cm²). The hair was irradiated for approximately 20 seconds during the curling process and approximately 30 seconds post-curl. As a control, the same procedure was performed on a second hair sample of equal size and type using a commercial styling product (Hot Set, Warren-Tricomi). The two hair samples were washed three times with water and the remaining curl was examined. The hair sample containing F2 showed dramatically increased curl retention. Additionally, the F2-hair sample demonstrated more elasticity and exhibited a much more natural feel than the hair treated with the commercial product.

The same procedure was used to straighten naturally wavy hair. F2 showed superior straightening, anti-frizzing, and humidity resistance compared to the commercial styling product (Hot Set, Warren-Tricomi).

In this example, the monomer trimethylolpropane triacrylate (5 to 50% w/w) and the thermal initiator benzoyl peroxide (0.1-1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the ability of the treated hair to resist humidity was tested. Benzoyl peroxide (1% w/w) at three different concentrations (5, 10, and 20% w/w) of trimethylolpropane triacrylate was dissolved in a commercial hair spray and compared to the commercial hair spray alone in its ability to retain a curl in humid conditions. The formulations were applied to hair and curled with a curling iron under standard conditions. They were then placed in a humidity chamber at 80-90% relative humidity for 1 hour. The formulations with 20% and 10% trimethylolpropane triacrylate showed a 2-fold increase in curl retention over the commercial product while the 5% trimethylolpropane triacrylate showed little difference.

The ability of the treated hair to resist static buildup was also tested. The prior formulations were applied to hair samples and were curled, brushed, and placed in a humidity chamber for 1 hour at 80-90% relative humidity. The samples were then agitated with latex gloves and the static effects were measured. The samples containing 10 and 20% trimethylolpropane triacrylate showed a 2-fold increase in anti-static properties.

In this example, the monomer tridecyl acrylate (0.1 to 50% w/w) and the thermal initiator benzoyl peroxide (0.1-2%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer di-trimethylolpropane tetraacrylate (0.1 to 50% w/w) and the thermal initiator benzoyl peroxide (0.1-2%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer SR9041(Sartomer) (0.1 to 50% w/w) and the thermal initiator benzoyl peroxide (0.1-2%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer trimethylolpropane triacrylate (0.1-50% w/w) and the thermal initiator lauryl peroxide (0.1-1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer tridecyl acrylate (0.1 to 50% w/w) and the thermal initiator lauryl peroxide (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer di-trimethylolpropane tetraacrylate (0.1 to 50% w/w) and the thermal initiator lauryl peroxide (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer SR9041(Sartomer) (0.1 to 50% w/w) and the thermal initiator lauryl peroxide (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer trimethylolpropane triacrylate (0.1 to 50% w/w) and the thermal initiator AIBN (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer tridecyl acrylate (0.1 to 50% w/w) and the thermal initiator AIBN (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer di-trimethylolpropane tetraacrylate (0.1 to 50% w/w) and the thermal initiator AIBN (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

In this example, the monomer SR9041(Sartomer) (0.1 to 50% w/w) and the thermal initiator AIBN (0.1 to 1%) are dissolved in denatured ethanol. This solution is applied to untreated hair and curled on a standard curling iron. The polymerization takes place within 30 seconds and the curler is removed. The hair samples are evaluated for beneficial properties compared to the commercial product and are shown to exhibit increased curl retention.

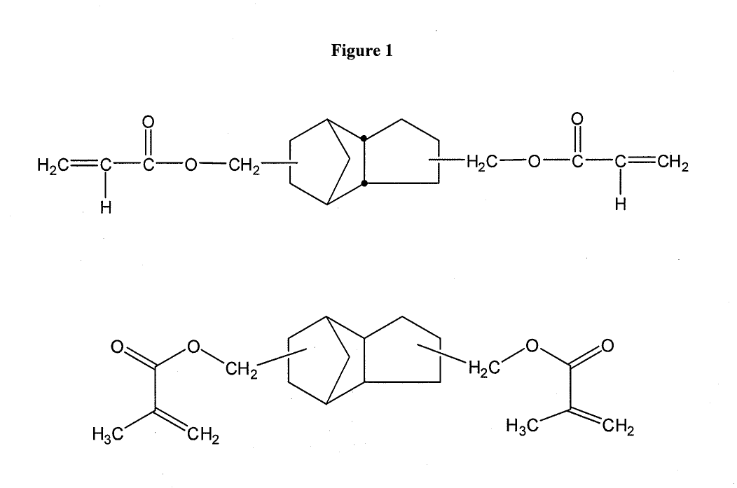

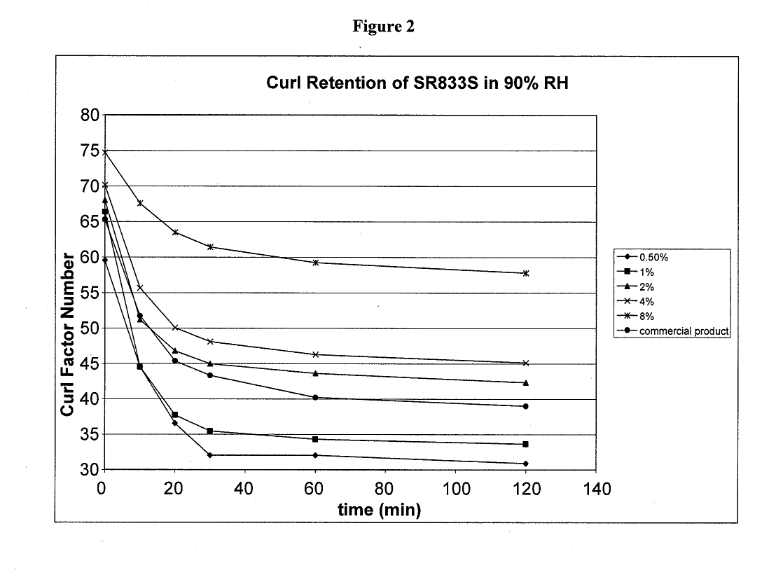

In this example, the monomer tricyclodecane dimethanol diacrylate (Sartomer SR833S) (0.1% to 8% w/w) is dissolved in denatured ethanol along with one or both of the thermal initiators AIBN (0.6% to 2% w/w) and BPO (1% to 2% w/w). This solution is applied to untreated, straight hair and curled with a curling iron. Monomer-treated hair samples are compared to a commercial styling product (Bumble and Bumble Styling Lotion) and shown to exhibit increased curl retention (FIGS. 1 and 2).

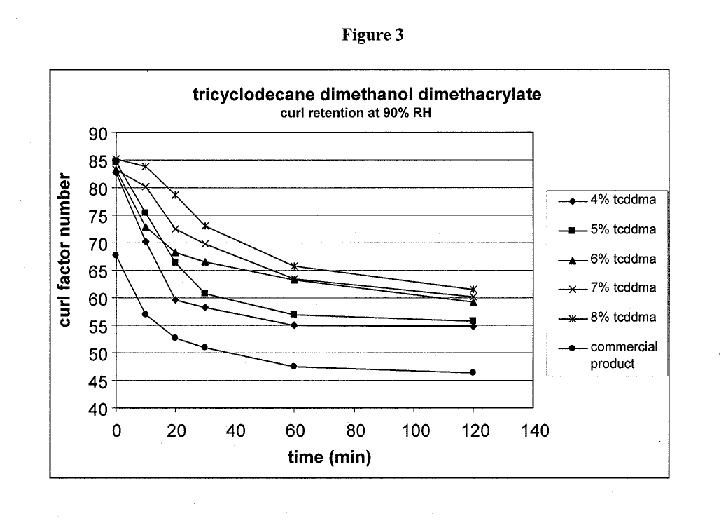

In this example, the monomer tricyclodecane dimethanol dimethacrylate (4% to 12% w/w) is dissolved in denatured ethanol with one or both of the thermal initiators AIBN (0.6% to 2% w/w) and BPO (1% to 2% w/w). Solutions are applied to untreated, straight hair and curled with a standard curling iron. Monomer-treated hair samples are compared to samples treated with a commercial product (Bumble and Bumble Styling Lotion) and shown to exhibit increased curl retention (FIGS. 1 and 3).

In this example, the monomer trimethylolpropane trimethacrylate (Sartomer SR350) (5% to 30% w/w) is dissolved in denatured ethanol with one or both of the thermal initiators AIBN (1% to 2% w/w) and BPO (1% to 2% w/w). Solutions are applied to untreated, straight hair and curled with a standard curling iron. These hair samples are compared to a sample that was curled with a commercial product (Bumble and Bumble Styling Lotion) and shown to exhibit increased curl retention.

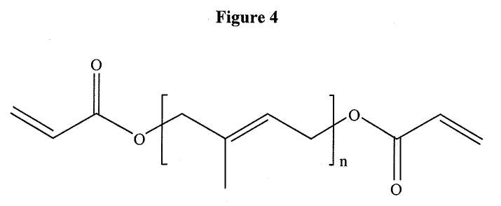

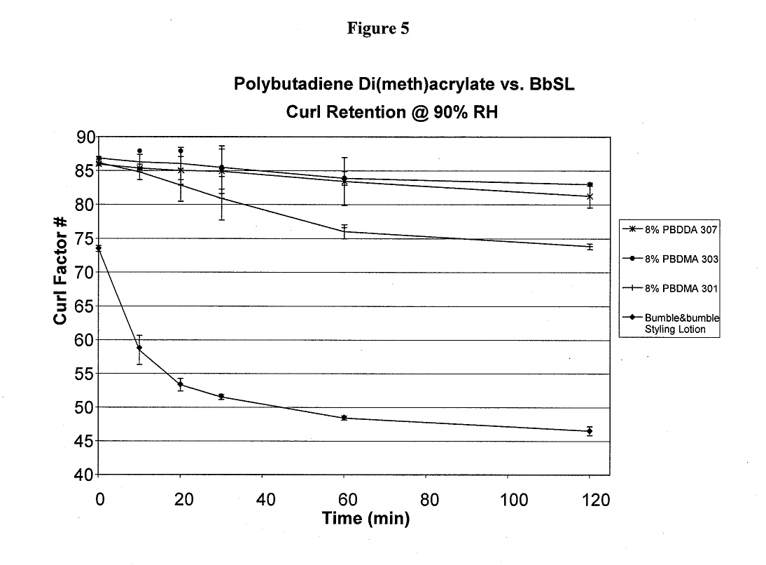

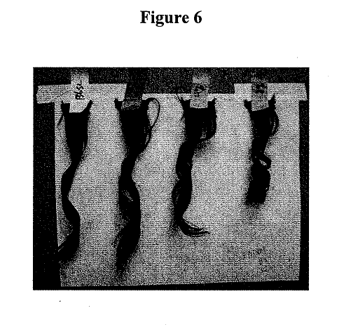

In this example, the monomer blend of polybutadiene dimethacrylate (80%) and 1,6-hexanediol diacrylate (20%) (Sartomer CN301) (0.1 to 8% w/w) and the thermal initiator BPO (0.1 to 2%) are dissolved in methyl acetate. This solution is applied to untreated straight hair and curled on a standard curling iron. The polymerization takes place within 30 seconds and the curler is removed. The hair samples are evaluated for beneficial properties compared to the commercial product and are shown to exhibit increased curl retention (FIGS. 4 and 5).

In this example, the monomer polybutadiene dimethacrylate (Sartomer CN303) (0.1 to 8% w/w) and the thermal initiators BPO (0.1 to 2%) and/or AIBN (0.1 to 2%) are dissolved in methyl, ethyl, propyl, butyl, or amyl acetate. This solution is applied to untreated straight hair and curled on a standard curling iron. The polymerization takes place within 30 seconds and the curler is removed. The hair samples are evaluated for beneficial properties compared to the commercial product and are shown to exhibit increased curl retention (FIGS. 4 and 5).

In this example, the monomer polybutadiene diacrylate (Sartomer CN307) (0.1 to 8% w/w) and the thermal initiator BPO (0.1 to 1%) are dissolved in denatured methyl acetate. This solution is applied to untreated straight hair and curled on a standard curling iron. The polymerization takes place within 30 seconds and the curler is removed. The hair samples are evaluated for beneficial properties compared to the commercial product and are shown to exhibit increased curl retention.(FIGS. 4 and 5).

In this example, the monomer polyisoprene diacrylate (San Esters PIDA) (0.1 to 8% w/w) and the thermal initiator BPO (0.1 to 1%) are dissolved in ethyl acetate. This solution is applied to untreated straight hair and curled on a standard curling iron. The polymerization takes place within 30 seconds and the curler is removed. The hair samples are evaluated for beneficial properties compared to the commercial product and are shown to exhibit increased curl retention (FIG. 4).

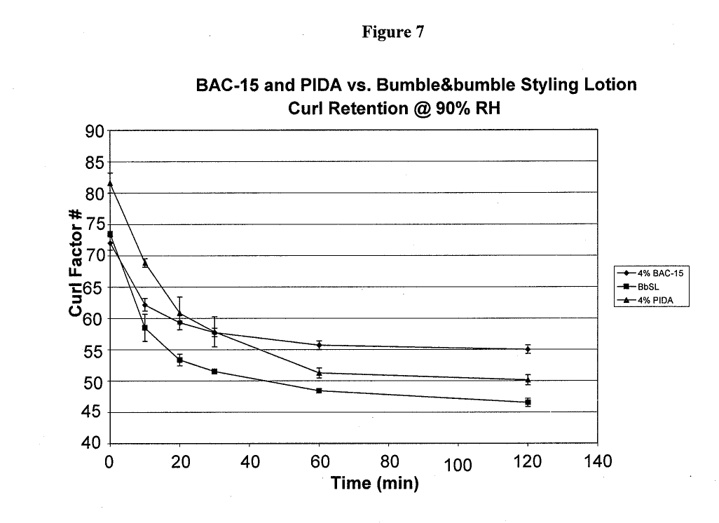

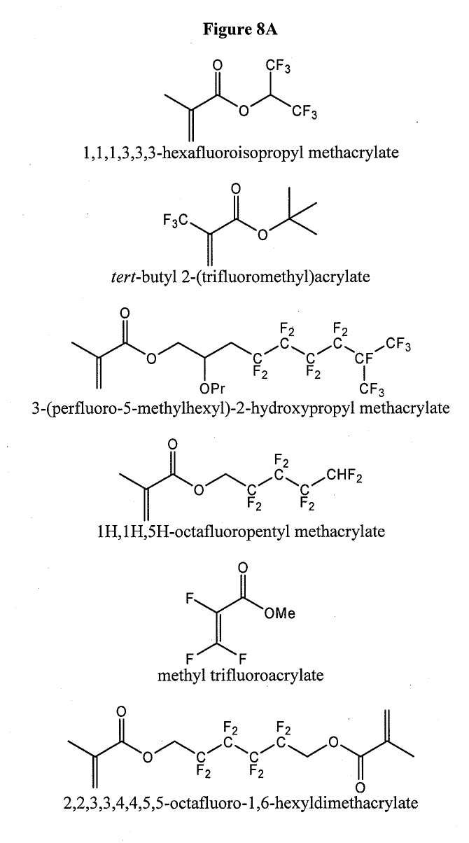

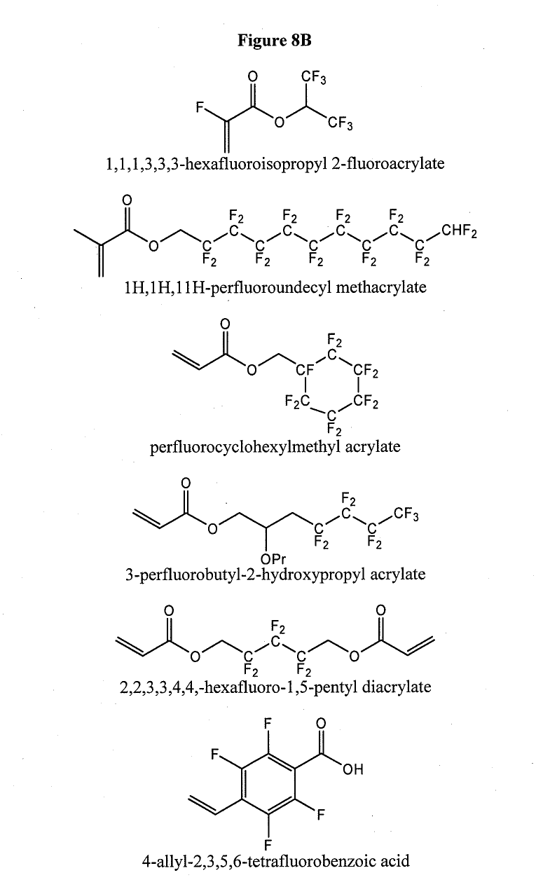

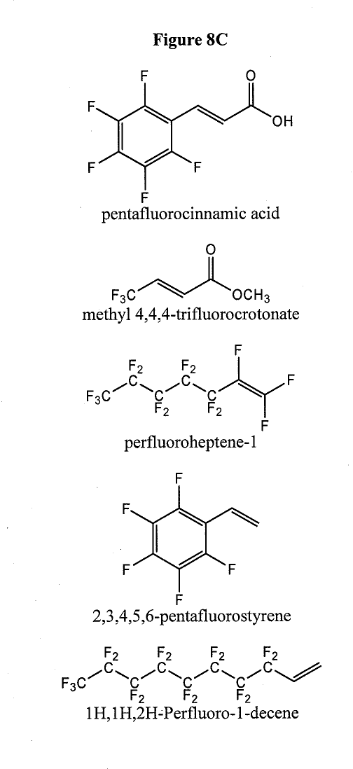

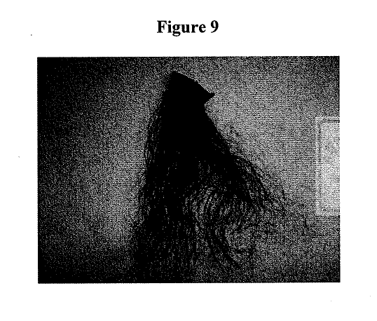

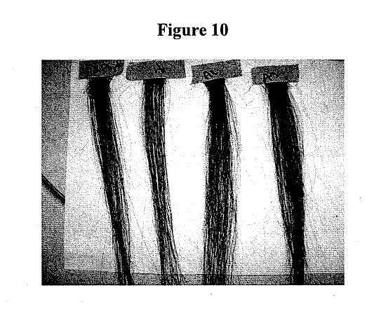

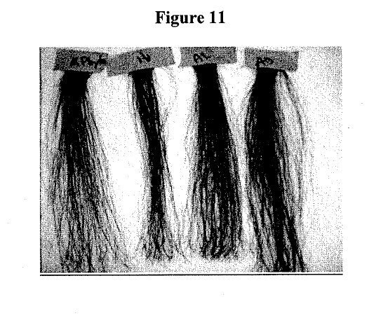

In this example, the monomer polybutadiene diacrylate (San Esters BAC-15) (0.1 to 50% w/w) and the thermal initiators BPO (0.1 to 1%) and/or AIBN (0.1 to 2%) are dissolved in ethyl acetate. This solution is applied to untreated straight hair and curled on a standard curling iron. The polymerization takes place within 30 seconds and the curler is removed. The hair samples are evaluated for beneficial properties compared to the commercial product and are shown to exhibit increased curl retention (FIG. 7).

In this example, a liquid solution containing 500 mg of 2,2,3,3,4,4,5,5-octafluoro-1,6-hexyl dimethacrylate and 100 mg of dibenzoyl peroxide in a mixture of 5.9 mL of ethanol and 3.5 mL methyl acetate was sprayed onto a swatch of coarse and curly (or frizzy) hair 2″ wide which is pinned to a dummy head. A standard pump spray bottle top was used to spray the formulation, and the hair was sprayed 5 to 6 times so that there was an even coating on the hair. Each spray puts about 0.14 mL of solution onto the hair. The hair was then “blown out” which involves wrapping the hair around a round brush and pulling it straight slowly while blowing on it with a blow drier (about 100° C.). Next, the hair was straightened with a ceramic flat iron heated to around 205° C. It takes roughly 2 to 3 passes with the iron followed by a comb to achieve the optimal results. The hair was left with a unique, frictionless feel. Hair samples treated this way were given an average score of 4.5 out of 5 for feel and frizz control by a panel of experts in a blind survey of hair samples. Some of these samples were treated with our formulation, while others were prepared with a variety of comparable commercial products, including Phyto Defrisant and Bumble&Bumble Styling Lotion. Commercial products scored between 3.5 and 4.5.

The treated hair was then placed into a humidity chamber (85% RH) for 45 minutes. Afterwards, the hair sample maintained its smooth feeling as well as its straightness.

A similarly treated hair sample was then rinsed for 30 seconds with warm water. The hair was blow-dried to dryness. After three cycles of this, the sample maintained smoothness and some degree of straightness. Another treated sample was shampooed with 0.5 mL of Johnson & Johnson Baby Shampoo for 30 seconds and then rinsed with warm water and blow-dried completely. After one cycle, smoothness and straightness were maintained, and after two cycles some smoothness and straightness still remained in the hair.

As a control, the procedures above were repeated replacing the formulation with water. After styling, the water control was soft, but lacked the sleek, coated feeling of the formulation. After one shampoo or one rinse, the water treated hair reverted back to its naturally curly and frizzy state. The water control frizzed out 20% more than the treated in the humidity test.

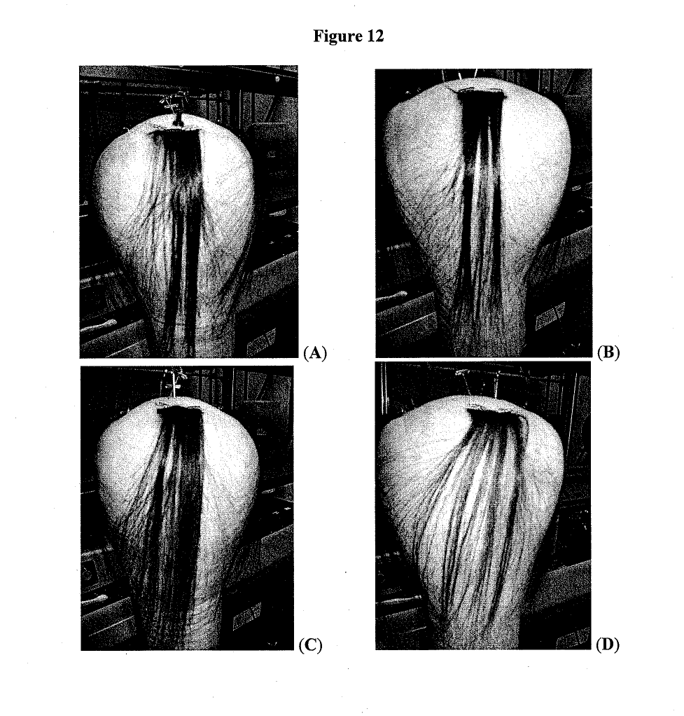

Alongside samples prepared with water, Bumble&Bumble Styling Lotion, and Phyto Defrisant, our formulation was brushed five times with a large vent brush to create static and induce frizz. Hair treated with our formulation showed an increase of about 5% in fly-away hair strands, while samples prepared with water and Bumble&Bumble Styling Lotion showed about 40% more fly-away hair strands and samples treated with Phyto Defrisant showed about 20% more fly-away hair strands (FIG. 12).

In this example, a liquid solution containing 500 mg of 2,2,3,3,4,4,5,5-octafluoro-1,6-hexyl dimethacrylate, 100 mg Esacure® EDB, and 100 mg of dibenzoyl peroxide in a mixture of 5.9 mL of ethanol and 3.5 mL of methyl acetate is sprayed onto a swatch of double-bleached sandy brown, straight hair. To bleach the hair, 35% hydrogen peroxide and Wella Blondor Whitening Powder were mixed in a dish and applied to sandy brown, straight hair with a brush. The hair was left wrapped in foil and heated for two minutes with a heat gun, rinsed with water, and then dried with a blow drier. The bleaching solution was then applied to the other side of the hair samples, wrapped in foil, and heated for 1 additional minute. A swatch 2″ wide of the damaged hair is pinned to a dummy head. A standard pump spray bottle top is used to spray the formulation, and the hair is sprayed 7 to 8 times so the hair is saturated with the formulation. Each spray puts about 0.14 mL of the formulation on the hair. The hair is then “blown out” which involves wrapping the hair around a round brush and pulling it straight slowly while blowing on it with a blow drier (about 100° C.). The hair that was once coarse, tangled, and dull becomes as smooth, manageable, and shiny as the hair before bleaching. Hair samples treated this way were given an average score of 4.75 out of 5 by a panel of experts in a blind survey of hair samples. The panel scored for feel, stylability, shine, and health. Some of these samples were treated with our formulation, while others were prepared with a variety of comparable commercial products, including Phyto Defrisant. Commercial products scored between 3.5 and 4.75.

The treated hair was then shampooed with 0.5 mL of Johnson & Johnson Baby Shampoo for 30 seconds and then rinsed with warm water and blow-dried completely. The rejuvenated quality of the hair was maintained after two cycles of this process, and at least 3 cycles of a water-rinse only process.

In this example, a liquid solution containing 200 mg of 1H,1H,2H-perfluoro-1-decene, 1.5 g water, and 100 mg of 2,2′-azobis(isobutyronitrile) in 8.2 g of ethanol was sprayed onto a swatch 2″ wide and ¼″ thick of coarse and curly and frizzy hair which is pinned to a dummy head. A standard spray bottle top was used to spray the formulation, and the hair was sprayed until it was completely saturated (15-20 sprays). The hair was then “blown out” which involves wrapping the hair around a round brush and pulling it straight slowly while blowing on it with a blow drier (about 100° C.). Next, the hair was straightened with a ceramic flat iron heated to around 205° C. It takes roughly 2 to 3 passes with the iron followed by a comb to achieve the optimal results. The hair was left with a unique smoothness without the characteristic greasy feel produced from a silicone product.

The effect of humidity, rinsing and shampooing on the feel and straightness was also examined. The treated hair (above) was then placed into a humidity chamber (85% RH) for 45 minutes. Afterwards, the hair sample maintained 90% of its smooth feeling as well as its straightness. In contrast, a control (water treated) frizzed out 20% more than the treated.

A similar treated hair sample was then rinsed for 30 seconds with warm water. The hair was blow-dried to dryness. After three cycles of rinsing and drying, the sample maintained smoothness and some degree of straightness. Another treated sample was shampooed for 30 seconds and then rinsed with warm water and blow-dried completely. After one cycle, smoothness and straightness were maintained (similar to three cycles of rinsing), and after two, some smoothness and straightness still remained in the hair. As a control, the procedures above were repeated treating the hair with water alone. After styling, the water control was soft, but lacked the sleek, coated feeling of the formulation. After one shampoo or one rinse, the control reverted back to its naturally curly and frizzy state.

In this example, a liquid solution containing 200 mg of 1H, 1H, 2H-perfluoro-1-decene, 1.5 g water, and 100 mg of 2,2′-azobis(isobutyronitrile) in 8.2 g of ethanol is sprayed onto a swatch 2″ wide and ¼″ thick of triple-bleached sandy brown, straight hair which is pinned to a dummy head. A standard spray bottle top is used to spray the formulation, and the hair is sprayed 15 to 20 times, or so the hair is saturated with the formulation. The hair is then “blown out” which involves wrapping the hair around a round brush and pulling it straight slowly while blowing on it with a blow drier (about 100° C.). In a blind test, panelists could easily distinguish between the untreated (damaged) hair and the treated (damaged) hair. However, the difference between the undamaged hair and the treated (damaged) was minimal.

The treated hair was then washed with shampoo for 30 seconds and rinsed with warm water. The hair was blow-dried to dryness. The rejuvenated quality of the hair was maintained after two cycles of this process, and at least 3 cycles of a rinse (water) only process.

In this example, a liquid solution containing 200 mg of 1H, 1H, 2H-perfluoro-1-decene, 1.5 g water, and 100 mg of 2,2′-azobis(isobutyronitrile) in 8.2 g of ethanol was sprayed onto a swatch 2″ wide and ¼″ thick of dull, matte brown hair which is pinned to a dummy head. A standard spray bottle top was used to spray the formulation, and the hair was sprayed 15 to 20 times, so that there was an even coating on the hair. The hair was then “blown out” which involves wrapping the hair around a round brush and pulling it straight slowly while blowing on it with a blow drier (about 100° C.). Next, the hair was straightened with a ceramic flat iron heated to around 205° C. It takes roughly 2 to 3 passes with the iron followed by a comb to achieve the optimal results. The hair then exhibited a profound increase (45%) in shine and glossiness without inducing a sticky or greasy feel.

In this example, a liquid solution containing 500 mg of 2,2,3,3,4,4,5,5-octafluoro-1,6-hexyl dimethacrylate and 100 mg of dibenzoyl peroxide in a mixture of 5.9 mL of ethanol and 3.5 mL of methyl acetate was sprayed onto a swatch of dull, matte brown hair 2″ wide which is pinned to a dummy head. A standard pump spray bottle top was used to spray the formulation, and the hair was sprayed 5 to 6 times so that there was an even coating on the hair. Each spray puts approximately 0.14 mL of the formulation on the hair. The hair was then “blown out” which involves wrapping the hair around a round brush and pulling it straight slowly while blowing on it with a blow drier (about 100° C.). Next, the hair was straightened with a ceramic flat iron heated to around 205° C. It takes roughly 2 to 3 passes with the iron followed by a comb to achieve the optimal results. The hair then exhibited an increase in shine and glossiness without inducing a sticky or greasy feel.

A liquid solution containing 400 mg of 2,2,3,3,4,4,5,5-octafluoro-1,6-hexyl diacrylate, 400 mg 1,6-hexanediol diacrylate, and 200 mg of 2,2′-azobisisobutyronitrile in a mixture of 5.5 mL of ethanol and 3.5 mL methyl acetate was sprayed onto a swatch of brown, curly hair 2″ wide which was pinned to a dummy head. A standard pump spray bottle top was used to spray the formulation, and the hair was sprayed 5 to 6 times so that there was an even coating on the hair. Each spray puts approximately 0.14 mL of the formulation on the hair. The hair was then “blown out” which involves wrapping the hair around a round brush and pulling it straight slowly while blowing on it with a blow drier (about 100° C.). The formulation cures completely at the temperature of the blow drier while maintaining manageability and avoiding formation of unwanted precipitates. The hair is left with a distinctive soft, frictionless feel and improved shape without any of the residues common with many blow dry activated hair products. Hair samples treated this way were given an average score of 4.75 out of 5 for feel by a panel of experts in a blind survey of hair samples, while others prepared with a variety of comparable commercial products (including Phyto Defrisant, Bumble&Bumble Straight, and Bumble&Bumble Styling Lotion) scored between 3.65 and 4.5.

### Example 2

### Testing of Treated Hair

In addition to the tests described above for assessing various properties of the treated hair, other tests may be used to test elasticity, shine/luster, break strength, and hair fiber thickness.

In this example, the measure of the hair's elasticity is proposed. A formulation would be applied to a hair sample and curled with a curling iron. One end of the hair sample would be attached to a fixed surface. The other end of the sample a weight was attached. The weight would be raised to a set height and released to extend the hair sample. This process would be repeated several times and the weight would be removed. The recoil of the hair sample would be measured and compared to the recoil of that of a hair sample treated with a commercial product.

In this example, the measure of the hair's shine/luster is proposed. After applying a formulation and curling and brushing a hair sample, the hair would be wound around a cylinder and placed under a lamp that mimics sunlight. The width of the cone of luster will be measure and compared with that of a commercial product.

In this example, the measure of the hair's break strength is proposed. Single hair fibers (treated and untreated) can be attached to an Instron which will pull at one end of the fiber, breaking the fiber at a certain force.

In this example, the measure of the hair fiber thickness is proposed. Cross sections of hair fibers (treated and untreated) can be examined and measured by microscopy.

In this example, the humidity resistance of the treated hair is proposed. This property can be measured by placing the styled hair tress in an atmosphere of high humidity.

In this example, the feel is proposed. The parameters of feel can be assessed for a given material on the hair fiber. Several parameters such as tack, slip, stiffness, smoothness, grease, and strength can be evaluated by a blind test of experts.

In this example, the measure of the hair's elasticity is proposed. A formulation would be applied to a hair sample and curled with a curling iron. One end of the hair sample would be attached to a fixed surface. The other end of the sample a weight was attached. The weight would be raised to a set height and released to extend the hair sample. This process would be repeated several times and the weight would be removed. The recoil of the hair sample would be measured and compared to the recoil of that of a hair sample treated with a commercial product.

In this example, the measure of the hair's shine/luster is proposed. After applying a formulation and curling and brushing a hair sample, the hair would be wound around a cylinder and placed under a lamp that mimics sunlight. The width of the cone of luster will be measure and compared with that of a commercial product.

In this example, the measure of the hair's break strength is proposed. Single hair fibers (treated and untreated) can be attached to an Instron which will pull at one end of the fiber, breaking the fiber at a certain force.

In this example, the measure of the hair fiber thickness is proposed. Cross sections of hair fibers (treated and untreated) can be examined and measured by microscopy.

In this example, the humidity resistance of the treated hair is proposed. This property can be measured by placing the styled hair tress in an atmosphere of high humidity.

In this example, the measure of feel is proposed. The parameters of feel can be assessed for a given material on the hair fiber. Several parameters such as tack, slip, stiffness, smoothness, grease, and strength can be evaluated by a blind test of experts.

In this example, the measure of the hair's elasticity is proposed. A formulation would be applied to a hair sample and curled with a curling iron. One end of the hair sample would be attached to a fixed surface. The other end of the sample a weight was attached. The weight would be raised to a set height and released to extend the hair sample. This process would be repeated several times and the weight would be removed. The recoil of the hair sample would be measured and compared to the recoil of that of a hair sample treated with a commercial product.

In this example, the measure of the hair's shine/luster is proposed. After applying a formulation and curling and brushing a hair sample, the hair would be wound around a cylinder and placed under a lamp that mimics sunlight. The width of the cone of luster will be measure and compared with that of a commercial product.

In this example, the measure of the hair's break strength is proposed. Single hair fibers (treated and untreated) can be attached to an Instron which will pull at one end of the fiber, breaking the fiber at a certain force.

In this example, the measure of the hair fiber thickness is proposed. Cross sections of hair fibers (treated and untreated) can be examined and measured by microscopy.

### OTHER EMBODIMENTS

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1.-39. (canceled)

40. A method of treating hair, the method comprising steps of:
applying a fluorinated monomer to hair of a subject;
applying a polymerization initiator to the hair; and
polymerizing the monomers in situ on the hair.

41.-43. (canceled)

44. The method of claim 40, wherein the fluorinated monomer is a fluorinated acrylate, fluorinated methacrylate, fluorinated dimethacrylate, fluorinated diacrylate, fluorinated triacrylate, fluorinated tetraacrylate, fluorinated alkene or fluorinated alkyne.

45.-52. (canceled)

53. The method of claim 40, wherein at least 25% of the total number of hydrogen atoms and fluorine atoms of the fluorinated monomer are fluorine atoms.

54.-57. (canceled)

58. The method of claim 40, wherein the step of applying a fluorinated monomer comprises spraying, brushing, dipping, soaking, or rubbing a solution of fluorinated monomer on hair of a subject.

59. The method of claim 40, wherein the step of applying a fluorinated monomer comprises applying a solution of fluorinated monomer in a solvent selected from the group consisting of water, ethanol, denatural ethanol, methanol, isopropanol, propylene glycol, and mixture thereof.

60. The method of claim 59, wherein the solution further comprises a polymerization initiator.

61. The method of claim 59, wherein the solution further comprises a propellant.

62.-65. (canceled)

66. The method of claim 40, wherein the polymerization initiator is a photoinitiator.

67. (canceled)

68. The method of claim 40, wherein the polymerization initiator is a thermal initiator.

69.-123. (canceled)

124. The method of claim 40, wherein the fluorinated monomer is of one of the formulae: 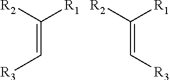
wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂, —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
wherein R₁, R₂, or R₃ comprises at least one fluorine atom.

125. The method of claim 40, wherein the fluorinated monomer is of formula: 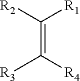
wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂, —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —C(═O)N(R_{D})₂; —CN; —SCN; —SR_{D}; —SOR_{D}; —SO₂R_{D}; —NO_{D}; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
wherein R₁, R₂, R₃, or R₄ comprises at least one fluorine atom.

126. The method of claim 125, wherein the fluorinated monomer is of the formula:

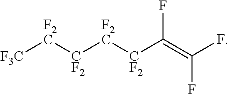

127. The method of claim 40, wherein the fluorinated monomer is of the formula: 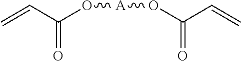
wherein A is a fluorinated linker.

128.-130. (canceled)

131. The method of claim 127, wherein linker A is of one of the formulae:

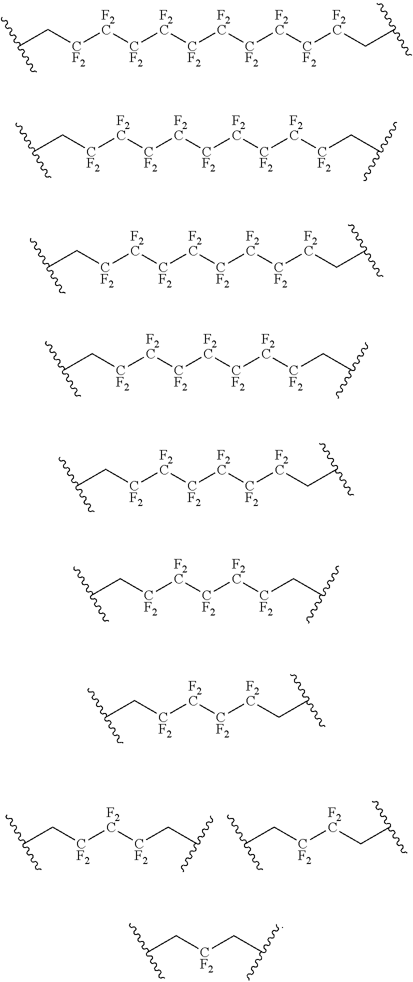

132. The method of claim 40, wherein the fluorinated monomer is of one of the formulae: 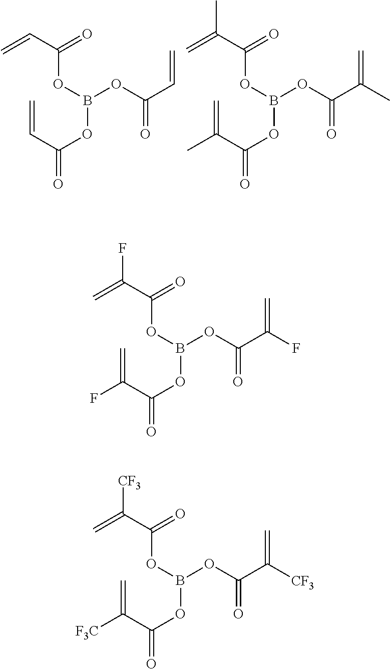
wherein B is fluorinated linker.

133. The method of claim 132, wherein the fluorinated monomer is of formula:

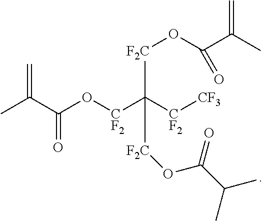

134. The method of claim 40, wherein the fluorinated monomer is of formula: 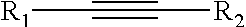
wherein
R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
wherein R₁ and R₂ comprises at least one fluorine atom.

135.-149. (canceled)
